(19) United States
(12) Patent Application Publication
(10) Pub. No.: US 2022/0193028 A1
(43) Pub. Date: Jun. 23, 2022

(54) THERAPEUTIC COMBINATIONS, LIQUID PHARMACEUTICAL COMPOSITIONS, KITS FOR THEIR PREPARATION, AND METHODS OF THEIR USE

(71) Applicant: PrevEp LLC, Bethesda, MD (US)

(72) Inventor: Chris RUNDFELDT, Coswig (DE)

(21) Appl. No.: 17/603,707

(22) PCT Filed: Apr. 17, 2020
(86) PCT No.: PCT/US2020/028777

§ 371 Date: Oct. 14, 2021

Related U.S. Application Data
(60) Provisional application No. 62/926,130, filed on Oct. 25, 2019.
(60) Provisional application No. 62/835,707, filed on Apr. 18, 2019.

(51) Int. Cl. A61K 31/36; A61K 47/26; A61K 31/4015; A61K 31/40; A61K 47/12; A61K 31/198; A61K 31/192; A61K 31/415; A61K 31/42; A61K 31/137; A61K 38/20; A61K 31/155; A61K 31/195; A61K 31/197; A61K 31/445; A61K 31/444; A61K 31/546; A61K 31/19; A61K 31/515; A61K 31/16; A61K 9/08; A61P 25/28; A61P 25/08

(52) U.S. Cl. CPC A61K 31/36; A61P 25/08; A61K 31/4015; A61K 31/40; A61K 47/12; A61K 31/198; A61K 31/192; A61K 31/415; A61K 31/42; A61K 31/137; A61K 38/2006; A61K 31/155; A61K 31/195; A61K 31/197; A61K 31/445; A61K 31/444; A61K 31/546; A61K 31/19; A61K 31/515; A61K 31/16; A61K 9/08; A61P 25/28; A61K 47/26

(57) ABSTRACT

Disclosed are liquid pharmaceutical compositions including topiramate or a pharmaceutically acceptable salt thereof, meglumine, and a pharmaceutically acceptable excipient. The liquid pharmaceutical composition may further include, e.g., levetiracetam or brivaracetam and/or atorvastatin or a pharmaceutically acceptable salt thereof (e.g., atorvastatin sodium).

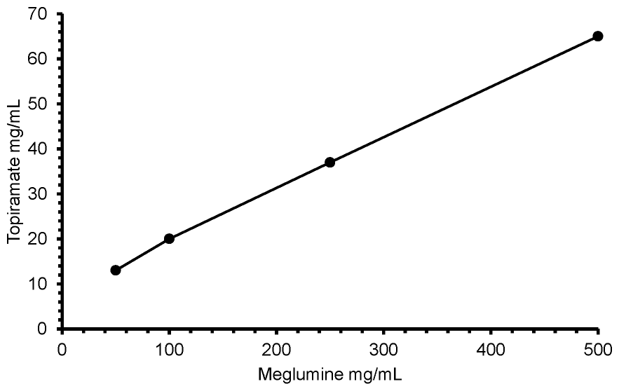

### FIELD OF THE INVENTION

The invention relates to liquid pharmaceutical compositions, kits for their preparation, and methods of their use.

### BACKGROUND

Pharmaceutical compositions provide a vehicle for therapeutic agent delivery. Parenteral pharmaceutical compositions are often liquid pharmaceutical compositions. For oral pharmaceutical dosing forms, liquid compositions can be also of advantage, as they allow for graded dosing by volume. In addition, they are useful in patients which are not able to swallow tablets or capsules or where a gastric tube allows for dosing of fluids only.

The need for liquid pharmaceutical compositions including multiple therapeutic agents is especially apparent for parenteral administration, as administration of multiple therapeutic agents in a single administration event may improve patient compliance and reduce waste generated from multiple parenteral administration devices. Accordingly, there is a need in the art for the development of liquid pharmaceutical compositions.

Topiramate, by way of example, is a prescription medication used as a monotherapy for epilepsy and is typically available in solid pharmaceutical compositions, such as tablets and sprinkle capsules. There are currently no United States Food and Drug Administration (U.S.F.D.A.)-approved parenteral or oral liquid pharmaceutical compositions of topiramate. Furthermore, there are currently no U.S.F.D.A-approved liquid (parenteral or oral) pharmaceutical compositions combining topiramate with levetiracetam or brivaracetam as dual combination or with levetiracetam or brivaracetam and atorvastatin as triple combination in a single liquid pharmaceutical composition. Thus, there is a need for parenteral or oral liquid compositions including topiramate or a pharmaceutically acceptable salt thereof for patients with limited ability to swallow tablets or capsules.

Epilepsy is a chronic disorder of the brain characterized by spontaneous recurrent seizures. Approximately 70 million people worldwide have epilepsy, making it one of the most common neurological diseases globally. Approximately 20% of all epilepsy is caused by acute brain insults such as traumatic brain injury (TBI), stroke, brain tumors, and infections. Acquired epilepsy is often difficult to treat and associated with comorbidities, e.g., depression, anxiety and cognitive deficits. The ability to prevent epilepsy after brain insult or reduce its severity is a great unmet need in neurology. Typically, an epileptogenic brain insult is followed by a seizure-free latent period during which a process, termed epileptogenesis, takes place that leads to the development of epilepsy. Epileptogenesis involves brain alterations such as blood-brain barrier disruption, inflammation, neurodegeneration, neuronal regeneration, sprouting of new fibers, synaptogenesis, and increased neuronal hyperexcitability and synchronization, ultimately leading to spontaneous recurrent seizures and comorbidities. An acute brain insult such as TBI, stroke or infection can induce epileptogenesis. The injury initiates processes, which ultimately lead to the development of chronic epilepsy and related symptoms (co-morbidities of epilepsy). After these injuries, epileptogenesis starts with a latent period, where no seizure activity can be recorded; at this time, the brain is remodeled, becoming epileptic. Clinical epilepsy develops after a latency that may be as short as a few days and as long as years. This latent period provides an opportunity to use treatment during this period to modify the epileptogenic process and prevent development of epilepsy, or to modify epilepsy to be less severe, or to treat or reduce the severity of co-morbidities.

Despite numerous previous clinical epileptogenesis treatment trials, to our knowledge, as yet no drug has been shown to treat epileptogenesis after brain insult. Drugs that failed in clinical epileptogenesis treatment trials include phenobarbital, phenytoin, their combination, carbamazepine, valproate, and magnesium sulfate. One likely explanation for the failure of previous clinical trials is that epilepsy is thought to be a network disease. Electrical kindling and status epilepticus (SE) are typically used to induce epileptogenesis in rodents. Kindling, and SE do not produce the same type of brain insult as TBI, cerebral stroke, or brain infection. Thus, the development or aggravation of epilepsy by kindling, SE or seizures can be expected to be different from the brain insult-induced epileptogenesis. Most of previous preclinical studies on potential antiepileptogenic drug effects have been performed in rat or mouse SE models induced by either chemical or electrical means in healthy animals. SE is a rare cause of acquired epilepsy in humans and almost never occurs in otherwise healthy individuals. Thus, preclinical data on disease-modifying or antiepileptogenic drug effects in post-SE models of acquired epilepsy are unlikely to be translated to the clinically relevant causes of epileptogenesis associated with a brain insult, e.g., TBI, stroke, or brain infection. For obtaining translatable preclinical data, drug experiments in models of TBI, stroke, or brain infection are needed. Few pharmacological studies (with single drug treatment) on epileptogenesis treatment in models of TBI, stroke, or brain infection are available, and, to our knowledge, none of these studies demonstrated any epileptogenesis treatment effect, although some disease modifying effect was observed in few studies.

Currently, there is no approved treatment for epileptogenesis after brain insult, e.g., TBI.

There is therefore a need for therapeutic approaches for treating epileptogenesis associated with a brain insult, e.g., TBI, stroke, or brain infection.

### SUMMARY OF THE INVENTION

In general, disclosed are liquid pharmaceutical compositions of topiramate or a pharmaceutically acceptable salt thereof.

In one aspect, disclosed are liquid pharmaceutical compositions including topiramate or a pharmaceutically acceptable salt thereof, meglumine, and a pharmaceutically acceptable excipient.

In some embodiments, the composition includes 1 mg/mL to 100 mg/mL (e.g., 1 mg/mL to 65 mg/mL, 10 mg/mL to 70 mg/mL, 20 mg/mL to 65 mg/mL, 4 mg/mL to 30 mg/mL, 4 mg/mL to 20 mg/mL, 9 mg/mL to 11 mg/mL, 4 mg/mL to 6 mg/mL) of topiramate or a pharmaceutically acceptable salt thereof.

Liquid pharmaceutical compositions disclosed herein may include, e.g., meglumine. In certain embodiments, the composition includes 1 mg/mL to 550 mg/mL (e.g., 1 mg/mL to 100 mg/mL, 1 mg/mL to 50 mg/mL, 5 mg/mL to 50 mg/mL, 10 mg/mL to 50 mg/mL, or 1 mg/mL to 30 mg/mL) of meglumine.

Liquid pharmaceutical compositions disclosed herein may include, e.g., levetiracetam. In particular embodiments, the composition includes 5 mg/mL to 500 mg/mL (e.g., 5 mg/mL to 150 mg/mL, 20 mg/mL to 150 mg/mL, 60 mg/mL to 120 mg/mL, or 30 mg/mL to 60 mg/mL) of levetiracetam. In further embodiments, the weight ratio of levetiracetam to topiramate to is 15:1 or less (e.g., 10:1 or less). In yet further embodiments, the weight ratio of levetiracetam to topiramate is at least 5:1. In other embodiments, the weight ratio of topiramate to atorvastatin is 5:1 to 15:1 (e.g., 5:1 to 10:1).

Liquid pharmaceutical compositions disclosed herein may include, e.g., brivaracetam. In still further embodiments, the composition includes 0.5 mg/mL to 50 mg/mL (e.g., 2 mg/mL to 8 mg/mL, 4 mg/mL to 8 mg/mL, or 2 mg/mL to 4 mg/mL) of brivaracetam. In other embodiments, the weight ratio of brivaracetam to topiramate is 1:1 or less. In yet other embodiments, the weight ratio of brivaracetam to topiramate to is at least 1:4. In certain embodiments, the weight ratio of brivaracetam to topiramate is 1:4 to 1:1.

Liquid pharmaceutical compositions disclosed herein may include, e.g., padsevonil. In still further embodiments, the composition includes 1 mg/mL to 100 mg/mL (e.g., 4 mg/mL to 16 mg/mL, 8 mg/mL to 16 mg/mL, or 4 mg/mL to 8 mg/mL) of padsevonil. In other embodiments, the weight ratio of padsevonil to topiramate is 2:1 or less. In yet other embodiments, the weight ratio of padsevonil to topiramate to is at least 1:4. In certain embodiments, the weight ratio of padsevonil to topiramate is 1:2 to 2:1.

Liquid pharmaceutical compositions disclosed herein may include, e.g., seletracetam. In still further embodiments, the composition includes 0.1 mg/mL to 40 mg/mL (e.g., 0.8 mg/mL to 4 mg/mL, 2.4 mg/mL to 4 mg/mL, or 1.2 mg/mL to 2 mg/mL) of seletracetam. In other embodiments, the weight ratio of topiramate to seletracetam is 10:1 or less. In particular embodiments, the weight ratio of topiramate to seletracetam is at least 2.5:1. In further embodiments, the weight ratio of topiramate to atorvastatin is 2.5:1 to 10:1.

Liquid pharmaceutical compositions disclosed herein may include, e.g., atorvastatin or a pharmaceutically acceptable salt thereof. In still other embodiments, the composition includes 0.1 mg/mL to 80 mg/mL (e.g., 0.1 mg/mL to 20 mg/mL, 0.1 mg/mL to 16 mg/mL, 0.2 mg/mL to 16 mg/mL, 0.4 mg/mL to 2 mg/mL, 1.2 mg/mL to 2 mg/mL, or 0.6 mg/mL to 1 mg/mL) of atorvastatin or a pharmaceutically acceptable salt thereof. In some embodiments, the weight ratio of topiramate to atorvastatin is 15:1 or less (e.g., 10:1 or less). In particular embodiments, the weight ratio of topiramate to atorvastatin is at least 5:1. In further embodiments, the weight ratio of topiramate to atorvastatin is 5:1 to 15:1 (e.g., 5:1 to 10:1). In certain embodiments, the composition includes atorvastatin sodium.

Liquid pharmaceutical compositions disclosed herein may include, e.g., a pharmaceutically acceptable excipient. In further embodiments, the composition further includes an acidulant (e.g., acetic acid). In yet further embodiments, the composition further includes an emulsifier (e.g., polyoxyethylene (20) sorbitan monooleate (polysorbate 80)). In still further embodiments, the composition includes 0.001% to 5.0% (w/v) of the emulsifier.

Liquid pharmaceutical compositions disclosed herein may include, e.g., a calcium-scavenging agent. In further embodiments, the calcium-scavenging agent is ethylenediaminetetraacetic acid (EDTA), egtazic acid (EGTA), BAPTA (1,2-bis(o-aminophenoxy)ethane-N,N,N′,N′-tetraacetic acid), or an alkali salt thereof. In still further embodiments, the composition includes 0.001% to 5.0% (w/v) of the calcium-scavenging agent (e.g., EDTA, EGTA, BAPTA, or an alkali salt thereof). A preferred concentration range for the calcium scavenging agent (e.g., EDTA, EGTA, BAPTA, or an alkali salt thereof) is 0.005 to 0.5% (w/v). An even more preferred concentration is 0.01 to 0.1% (w/v). Some calcium-scavenging agents may serve a multifunctional role: first, they may be used to scavenge calcium ions and, second, they may function as acidulants. Advantageously, in some embodiments, the liquid pharmaceutical compositions including a calcium-scavenging agent may include reduced quantities of an emulsifier or, in some instance, no emulsifier. Reduction or elimination of the emulsifier in the compositions is advantageous because of the typically unfavorable toxicological profiles of emulsifiers.

In other embodiments, the composition has a pH of 5.5 to 8.8 (e.g., 6.5 to 8.5).

In yet other embodiments, the composition is formulated for parenteral administration (e.g., for intravenous administration, subcutaneous administration, or intramuscular administration). In still other embodiments, the composition is formulated for oral administration.

In some embodiments, the composition is a dosage form. In certain embodiments, the composition is aqueous.

In another aspect, disclosed are kits including a first container and a second container. The first container including topiramate or a pharmaceutically acceptable salt thereof. The second container including a pharmaceutically acceptable aqueous solution of meglumine.

Typically, a combination of the contents of the first container and a second container may produce, e.g., a liquid pharmaceutical composition described herein.

In some embodiments, topiramate or a pharmaceutically acceptable salt thereof is present in the first container in a crystalline, micronized form. In particular embodiments, topiramate or a pharmaceutically acceptable salt thereof is present in an amount sufficient to produce a composition including 1 mg/mL to 65 mg/mL of topiramate upon combination with the pharmaceutically acceptable aqueous solution of meglumine.

In certain embodiments, the pharmaceutically acceptable aqueous solution includes 1 mg/mL to 550 mg/mL of meglumine.

In further embodiments, the first container further includes levetiracetam. In yet further embodiments, levetiracetam is present in an amount sufficient to produce a composition including 5 mg/mL to 500 mg/mL of levetiracetam upon combination with the pharmaceutically acceptable aqueous solution of meglumine. In still further embodiments, the weight ratio of levetiracetam to topiramate is 15:1 or less (e.g., 10:1 or less). In other embodiments, the weight ratio of levetiracetam to topiramate is at least 5:1. In other embodiments, the weight ratio of topiramate to atorvastatin is 5:1 to 15:1 (e.g., 5:1 to 10:1).

In further embodiments, the first container further includes brivaracetam. In still further embodiments, brivaracetam is present in an amount sufficient to produce a composition including 0.5 mg/mL to 50 mg/mL of brivaracetam upon combination with the pharmaceutically acceptable aqueous solution of meglumine. In still further embodiments, the weight ratio, the weight ratio of brivaracetam to topiramate to is 1:1 or less. In yet other embodiments, the weight ratio of brivaracetam to topiramate is at least 1:4. In certain embodiments, the weight ratio of brivaracetam to topiramate is 1:4 to 1:1.

In further embodiments, the first container further includes padsevonil. In still further embodiments, padsevonil is present in an amount sufficient to produce a composition including 1 mg/mL to 100 mg/mL (e.g., 4 mg/mL to 16 mg/mL, 8 mg/mL to 16 mg/mL, or 4 mg/mL to 8 mg/mL) of padsevonil upon combination with the pharmaceutically acceptable aqueous solution of meglumine. In still further embodiments, the weight ratio, the weight ratio of padsevonil to topiramate to is 2:1 or less. In yet other embodiments, the weight ratio of padsevonil to topiramate is at least 1:2. In certain embodiments, the weight ratio of padsevonil to topiramate is 1:2 to 2:1.

In further embodiments, the first container further includes seletracetam. In still further embodiments, seletracetam is present in an amount sufficient to produce a composition including 0.1 mg/mL to 40 mg/mL (e.g., 0.8 mg/mL to 4 mg/mL, 2.4 mg/mL to 4 mg/mL, or 1.2 mg/mL to 2 mg/mL) of seletracetam upon combination with the pharmaceutically acceptable aqueous solution of meglumine. In still further embodiments, the weight ratio of topiramate to seletracetam is 10:1 or less. In particular embodiments, the weight ratio of topiramate to seletracetam is at least 2.5:1. In further embodiments, the weight ratio of topiramate to atorvastatin is 2.5:1 to 10:1.

In yet other embodiments, the first container further includes atorvastatin or a pharmaceutically acceptable salt thereof. In still other embodiments, atorvastatin or a pharmaceutically acceptable salt thereof is present in an amount sufficient to produce a composition including 0.1 mg/mL to 80 mg/mL atorvastatin or a pharmaceutically acceptable salt thereof upon combination with the pharmaceutically acceptable aqueous solution of meglumine. In some embodiments, the weight ratio of topiramate to atorvastatin is 15:1 or less (e.g., 10:1 or less). In certain embodiments, the weight ratio of topiramate to atorvastatin is at least 5:1. In further embodiments, the weight ratio of topiramate to atorvastatin is 5:1 to 15:1 (e.g., 5:1 to 10:1). In particular embodiments, the composition includes atorvastatin sodium.

In further embodiments, the first container contents and the second container contents upon combination produce a composition having a pH of 5.5 to 8.8 (e.g., 6.5 to 8.5).

In yet further embodiments, the kit further includes an acidulant (e.g., acetic acid). In still further embodiments, the pharmaceutically acceptable aqueous solution of meglumine includes the acidulant.

In other embodiments, the kit further includes an emulsifier (e.g., polyoxyethylene (20) sorbitan monooleate (polysorbate 80)). In yet other embodiments, the pharmaceutically acceptable aqueous solution of meglumine includes the emulsifier. In still other embodiments, the emulsifier is present in an amount sufficient to produce a composition including 0.001% to 5.0% (w/v) of the emulsifier upon combination of the first container contents and the second container contents.

In further embodiments, the kit further includes a calcium-scavenging agent. In yet further embodiments, the calcium-scavenging agent is ethylenediaminetetraacetic acid (EDTA), egtazic acid (EGTA), BAPTA (1,2-bis(o-aminophenoxy)ethane-N,N,N′,N′-tetraacetic acid), or an alkali salt thereof. In still further embodiments, the calcium-scavenging agent is present in an amount sufficient to produce a composition including 0.001% to 5.0% (w/v) of the calcium scavenging agent (e.g., EDTA, EGTA, BAPTA, or an alkali salt thereof). A preferred concentration range for the calcium scavenging agent (e.g., EDTA, EGTA, BAPTA, or an alkali salt thereof) is 0.005 to 0.5% (w/v). An even more preferred concentration is 0.01 to 0.1% (w/v).

In some embodiments, a combination of the first container contents and the second container contents produces a composition for parenteral administration. In certain embodiments, in combination of the first container contents and the second container contents produces a composition for intravenous administration, subcutaneous administration, or intramuscular administration. In particular embodiments, a combination of the first container contents and the second container contents produces a composition for oral administration. In further embodiments, a combination of the first container contents and the second container contents produces a dosage form.

In yet further embodiments, the first container contents are solid (e.g., lyophilized).

In yet another aspect, disclosed are methods of treating a patient in need thereof by administering to the patient a therapeutically effective amount of the liquid pharmaceutical composition disclosed herein.

In still another aspect, disclosed are methods of treating a patient in need thereof by combining the first container contents and the second container contents in the kit to produce a liquid pharmaceutical composition, and administering a therapeutically effective amount of the liquid pharmaceutical composition to the patient.

In some embodiments, the therapeutically effective amount is 0.5 mg/kg/day to 20 mg/kg/day (e.g., 2 mg/kg/day to 15 mg/kg/day) of topiramate. In certain embodiments, the therapeutically effective amount is at least 40 mg/day (e.g., at least 200 mg/day) of topiramate. In particular embodiments, the therapeutically effective amount is 1200 mg/day or less (e.g., 400 mg/day or less) of topiramate.

In further embodiments, the therapeutically effective amount is 2.5 mg/kg/day to 150 mg/kg/day (e.g., 10 mg/kg/day to 75 mg/kg/day) of levetiracetam.

In yet further embodiments, the therapeutically effective amount is 0.2 mg/kg/day to 10 mg/kg/day (e.g., 0.5 mg/kg/day to 5 mg/kg/day) of brivaracetam.

In still further embodiments, the therapeutically effective amount is 0.1 to 1.5 mg/kg/day (e.g., 0.2 to 2.0 mg/kg/day) of atorvastatin.

In other embodiments, the liquid pharmaceutical composition is administered parenterally (e.g., intravenously, subcutaneously, or intramuscularly). In yet other embodiments, the liquid pharmaceutical composition is administered orally.

In still other embodiments, the patient is in need of a treatment for a disorder or condition selected from the group consisting of epilepsy, seizures, anoxia, stroke, traumatic brain injury, brain infection, brain abscess, aneurysm, subarachnoid hemorrhage, status epilepticus, refractory status epilepticus, refractory partial onset seizures (POS), addiction (e.g., gambling addiction or drug addiction), migraines, substance dependence, alcoholism, cocaine dependence, opioid dependence, nicotine dependence, metabolic syndrome X, diabetes mellitus, type 2, vomiting, obsessive-compulsive disorder, refractory generalized social phobia, Tourette Syndrome, levodopa-induced dyskinesia in Parkinson's Disease, Prader-Willi syndrome, multiple sclerosis, Lennox-Gastaut Syndrome, Dravet's syndrome, bipolar disorder, obesity, post-traumatic stress disorder, headaches (e.g., cluster headaches or severe headaches), and conditions caused by exposure to a chemical warfare nerve agent.

In some embodiments, the patient is in need of neuroprotection. In such embodiments, the patient is in need of a treatment for a disorder or condition selected from the group consisting of traumatic brain injury, stroke, a brain infection, and subarachnoid hemorrhage. In particular embodiments, the patient is in need of a treatment for traumatic brain injury. In further embodiments, the patient is in need of a treatment for stroke. In yet further embodiments, the patient is in need of a treatment for a brain infection. In still further embodiments, the patient is in need of a treatment for encephalitis, meningoencephalitis, or a brain abscess.

In one aspect, the invention provides a method of treating epileptogenesis in a subject after brain insult by administering to the subject a therapeutically effective amount of a therapeutic combination including two to five drugs selected from the group consisting of anti-inflammatory drugs, antioxidant drugs, neuroprotective drugs, GABA-potentiating drugs, glutamate-suppressing drugs, drugs with presynaptic effects on neuronal excitability, drugs having a metabolic mode of action, and pharmaceutically acceptable salts thereof, provided that the two to five drugs are all different.

In some embodiments, each anti-inflammatory drug is independently ibuprofen, celecoxib, parecoxib, a sartan, atorvastatin, fingolimod, anakinra, or agmatine. In certain embodiments, each antioxidant drug is independently α-tocopherol, deferoxamine, N-acetylcysteine, sulforaphane, or melatonin. In particular embodiments, each neuroprotective drug is independently gabapentin, pregabalin, ifenprodil, perampanel, memantine, agmatine, celecoxib, or ceftriaxone. In further embodiments, each GABA-potentiating drug and each glutamate-suppressing drug is independently topiramate, valproate, phenobarbital, deferoxamine, ceftriaxone, ifenprodil, perampanel, padsevonil, or memantine. In yet further embodiments, each drug with presynaptic effects on neuronal excitability is independently levetiracetam, brivaracetam, etiracetam, padsevonil, gabapentin, pregabalin, or valproate. In still further embodiments, each drug having a metabolic mode of action is independently stiripentol, 2-deoxy-D-glucose, 5-azacitidine, decitabine, β-hydroxybutyrate, or vorinostat.

In certain embodiments, the therapeutic combination is a combination of topiramate, levetiracetam, and deferoxamine or a pharmaceutically acceptable salt thereof. In particular embodiments, the therapeutic combination is a combination of topiramate, levetiracetam, and atorvastatin or a pharmaceutically acceptable salt thereof. In some embodiments, the therapeutic combination is a combination of topiramate and levetiracetam. In further embodiments, the therapeutic combination is a combination of topiramate, levetiracetam, and ceftriaxone or a pharmaceutically acceptable salt thereof. In yet further embodiments, the therapeutic combination is a combination of topiramate, levetiracetam, and gabapentin or a pharmaceutically acceptable salt thereof. In still further embodiments, the therapeutic combination is a combination of topiramate, levetiracetam, and pregabalin or a pharmaceutically acceptable salt thereof. In other embodiments, the therapeutic combination is s a combination of levetiracetam, topiramate, and α-tocopherol. In yet other embodiments, the therapeutic combination is a combination of levetiracetam, deferoxamine or a pharmaceutically acceptable salt thereof, and melatonin. In still other embodiments, the therapeutic combination is a combination of levetiracetam, deferoxamine or a pharmaceutically acceptable salt thereof, and celecoxib. In some embodiments, the therapeutic combination is a combination of levetiracetam, deferoxamine or a pharmaceutically acceptable salt thereof, gabapentin or a pharmaceutically acceptable salt thereof, and fingolimod or a pharmaceutically acceptable salt thereof. In certain embodiments, the therapeutic combination is a combination of levetiracetam, atorvastatin or a pharmaceutically acceptable salt thereof, and ceftriaxone. In particular embodiments, the therapeutic combination is a combination of levetiracetam and perampanel or a pharmaceutically acceptable salt thereof. In further embodiments, the therapeutic combination is a combination of levetiracetam, perampanel or a pharmaceutically acceptable salt thereof, and ceftriaxone. In yet further embodiments, the therapeutic combination is a combination of levetiracetam, parecoxib, and anakinra. In still further embodiments, the therapeutic combination is a combination of levetiracetam and phenobarbital.

In other embodiments, the therapeutic combination is a combination of brivaracetam and topiramate. In yet other embodiments, the therapeutic combination is a combination of brivaracetam, topiramate, and ceftriaxone. In still other embodiments, the therapeutic combination is a combination of brivaracetam and perampanel or a pharmaceutically acceptable salt thereof. In some embodiments, the therapeutic combination is a combination of brivaracetam, perampanel or a pharmaceutically acceptable salt thereof, and ceftriaxone. In certain embodiments, the therapeutic combination is a combination of topiramate, brivaracetam, and deferoxamine or a pharmaceutically acceptable salt thereof. In particular embodiments, the therapeutic combination is a combination of topiramate, brivaracetam, and atorvastatin or a pharmaceutically acceptable salt thereof. In yet further embodiments, the therapeutic combination is a combination of topiramate, brivaracetam, and gabapentin or a pharmaceutically acceptable salt thereof. In still further embodiments, the therapeutic combination is a combination of topiramate, brivaracetam, and pregabalin or a pharmaceutically acceptable salt thereof. In other embodiments, the therapeutic combination is s a combination of brivaracetam, topiramate, and α-tocopherol. In yet other embodiments, the therapeutic combination is a combination of brivaracetam, deferoxamine or a pharmaceutically acceptable salt thereof, and melatonin. In still other embodiments, the therapeutic combination is a combination of brivaracetam, deferoxamine or a pharmaceutically acceptable salt thereof, and celecoxib. In some embodiments, the therapeutic combination is a combination of brivaracetam, deferoxamine or a pharmaceutically acceptable salt thereof, gabapentin or a pharmaceutically acceptable salt thereof, and fingolimod or a pharmaceutically acceptable salt thereof. In certain embodiments, the therapeutic combination is a combination of brivaracetam, atorvastatin or a pharmaceutically acceptable salt thereof, and ceftriaxone. In yet further embodiments, the therapeutic combination is a combination of brivaracetam, parecoxib, and anakinra. In still further embodiments, the therapeutic combination is a combination of brivaracetam and phenobarbital.

In other embodiments, the therapeutic combination is a combination of padsevonil and topiramate. In yet other embodiments, the therapeutic combination is a combination of padsevonil, topiramate, and ceftriaxone. In still other embodiments, the therapeutic combination is a combination of padsevonil and perampanel or a pharmaceutically acceptable salt thereof. In some embodiments, the therapeutic combination is a combination of padsevonil, perampanel or a pharmaceutically acceptable salt thereof, and ceftriaxone. In certain embodiments, the therapeutic combination is a combination of topiramate, padsevonil, and deferoxamine or a pharmaceutically acceptable salt thereof. In particular embodiments, the therapeutic combination is a combination of topiramate, padsevonil, and atorvastatin or a pharmaceutically acceptable salt thereof. In yet further embodiments, the therapeutic combination is a combination of topiramate, padsevonil, and gabapentin or a pharmaceutically acceptable salt thereof. In still further embodiments, the therapeutic combination is a combination of topiramate, padsevonil, and pregabalin or a pharmaceutically acceptable salt thereof. In other embodiments, the therapeutic combination is s a combination of padsevonil, topiramate, and α-tocopherol. In yet other embodiments, the therapeutic combination is a combination of padsevonil, deferoxamine or a pharmaceutically acceptable salt thereof, and melatonin. In still other embodiments, the therapeutic combination is a combination of padsevonil, deferoxamine or a pharmaceutically acceptable salt thereof, and celecoxib. In some embodiments, the therapeutic combination is a combination of padsevonil, deferoxamine or a pharmaceutically acceptable salt thereof, gabapentin or a pharmaceutically acceptable salt thereof, and fingolimod or a pharmaceutically acceptable salt thereof. In certain embodiments, the therapeutic combination is a combination of padsevonil, atorvastatin or a pharmaceutically acceptable salt thereof, and ceftriaxone. In yet further embodiments, the therapeutic combination is a combination of padsevonil, parecoxib, and anakinra. In still further embodiments, the therapeutic combination is a combination of padsevonil and phenobarbital.

In other embodiments, the therapeutic combination is a combination of seletracetam and topiramate. In yet other embodiments, the therapeutic combination is a combination of seletracetam, topiramate, and ceftriaxone. In still other embodiments, the therapeutic combination is a combination of seletracetam and perampanel or a pharmaceutically acceptable salt thereof. In some embodiments, the therapeutic combination is a combination of seletracetam, perampanel or a pharmaceutically acceptable salt thereof, and ceftriaxone. In certain embodiments, the therapeutic combination is a combination of topiramate, seletracetam, and deferoxamine or a pharmaceutically acceptable salt thereof. In particular embodiments, the therapeutic combination is a combination of topiramate, seletracetam, and atorvastatin or a pharmaceutically acceptable salt thereof. In yet further embodiments, the therapeutic combination is a combination of topiramate, seletracetam, and gabapentin or a pharmaceutically acceptable salt thereof. In still further embodiments, the therapeutic combination is a combination of topiramate, seletracetam, and pregabalin or a pharmaceutically acceptable salt thereof. In other embodiments, the therapeutic combination is s a combination of seletracetam, topiramate, and α-tocopherol. In yet other embodiments, the therapeutic combination is a combination of seletracetam, deferoxamine or a pharmaceutically acceptable salt thereof, and melatonin. In still other embodiments, the therapeutic combination is a combination of seletracetam, deferoxamine or a pharmaceutically acceptable salt thereof, and celecoxib. In some embodiments, the therapeutic combination is a combination of seletracetam, deferoxamine or a pharmaceutically acceptable salt thereof, gabapentin or a pharmaceutically acceptable salt thereof, and fingolimod or a pharmaceutically acceptable salt thereof. In certain embodiments, the therapeutic combination is a combination of seletracetam, atorvastatin or a pharmaceutically acceptable salt thereof, and ceftriaxone. In yet further embodiments, the therapeutic combination is a combination of seletracetam, parecoxib, and anakinra. In still further embodiments, the therapeutic combination is a combination of seletracetam and phenobarbital.

In certain embodiments, the therapeutic combination is a combination of valproate or a pharmaceutically acceptable salt thereof, losartan or a pharmaceutically acceptable salt thereof, and memantine or a pharmaceutically acceptable salt thereof.

In particular embodiments, the therapeutic combination comprises three of the drugs. In further embodiments, the dose of each drug in the combination is the highest tolerable dose in the individual patient, when the drug is administered in the therapeutic combination. In yet further embodiments, agmatine is administered at a dose of 3.56 g/day. In still further embodiments, anakinra is administered at a dose of 8 mg/kg/day. In some embodiments, brivaracetam is administered at a dose of 400 mg/day. In certain embodiments, padsevonil is administered at a dose of 800 mg/day. In particular embodiments, ceftriaxone is administered at a dose of 4 g/day. In further embodiments, deferoxamine is administered at a dose of 62 mg/kg/day up to a maximum of 6000 mg/day. In yet further embodiments, fingolimod is administered at a dose of 1.25 mg/day. In still further embodiments, gabapentin is administered at a dose of 3,200 mg/day. In other embodiments, ifenprodil is administered at a dose of 40 mg/day. In yet other embodiments, levetiracetam is administered at a dose of 55 mg/kg up to a maximum of 6,000 mg/day. In still other embodiments, losartan is administered at a dose of 100 mg/day. In some embodiments, melatonin is administered at a dose of 20 mg/day. In certain embodiments, memantine is administered at a dose of 28 mg/day. In particular embodiments, N-acetylcysteine is administered at a dose of 300 mg/kg/day. In further embodiments, perampanel is administered at a dose of 24 mg/day. In yet further embodiments, phenobarbital is administered at a dose of 200 mg. In still further embodiments, sulforaphane is administered at a dose of 60 mg/day. In other embodiments, topiramate is administered at dose of 400 mg/day. In yet other embodiments, valproate is administered at a dose of 3,000 mg/day. In still other embodiments, α-tocopherol is administered at a dose of 15 mg/day.

In some embodiments, the therapeutically effective dose represents 50% to 75% of the highest approved dose for administration to a human as a monotherapy. In certain embodiments, agmatine is administered at a dose of 1.78-2.67 g/day. In particular embodiments, anakinra is administered at a dose of 4-6 mg/kg/day. In further embodiments, brivaracetam is administered at a dose of 200-300 mg/day. In yet further embodiments, padsevonil is administered at a dose of 400 mg/day. In still further embodiments, ceftriaxone is administered at a dose of 2-3 g/day. In other embodiments, deferoxamine is administered at a dose of 31-4500 mg/kg/day. In yet other embodiments, fingolimod is administered at a dose of 0.625-0.94 mg/day. In still other embodiments, gabapentin is administered at a dose of 1600-2400 mg/day. In some embodiments, ifenprodil is administered at a dose of, 20-30 mg/day. In certain embodiments, levetiracetam is administered at a dose of 27.5-41 mg/kg/day up to a maximum of 3000-4500 mg/day. In particular embodiments, losartan is administered at a dose of 50-75 mg/day. In further embodiments, melatonin is administered at a dose of 10-15 mg/day. In yet further embodiments, memantine is administered at a dose of 14-21 mg/day. In still further embodiments, N-acetylcysteine is administered at a dose of 150-225 mg/kg/day. In some embodiments, perampanel is administered at a dose of 12-18 mg/day. In certain embodiments, phenobarbital is administered at a dose of 100-150 mg/day. In particular embodiments, sulforaphane is administered at a dose of 30-45 mg/day. In further embodiments, topiramate is administered at a dose of 200-300 mg/day. In yet further embodiments, valproate is administered at a dose of 1500-2250 mg/day. In still further embodiments, α-tocopherol is administered at a dose of 7.5-11.25 mg/day.

In certain embodiments, the therapeutic combination has a tolerability and efficacy that are greater than the expected additive tolerability and efficacy of the drugs in the therapeutic combination. In particular embodiments, the therapeutic combination is initially administered to the subject intravenously for 1 to 30 days. In certain embodiments, the therapeutic combination is administered to the subject intramuscularly, subcutaneously, orally, percutaneously, sublingually, buccally, intranasally, by inhalation, or rectally. In further embodiments, after the therapeutic combination administration is initiated, the therapeutic combination administration is maintained by prolonged oral or parenteral administration. In yet further embodiments, the therapeutic combination administration is maintained for 3 to 6 months following the brain insult. In still further embodiments, at least two drugs are present in the same pharmaceutical composition. In other embodiments, at least three drugs are present in the same pharmaceutical composition. In yet other embodiments, the administering step is initiated within 7 days after the brain insult. In still other embodiments, the administering step is initiated within 48 h after the brain insult. In some embodiments, the administering step is initiated within 24 h after the brain insult. In particular embodiments, the administering step is initiated within 8 h after the brain insult. In certain embodiments, the therapeutic combination is administered to the subject for a period of 3 day to 3 months after the insult. In further embodiments, the therapeutic combination is administered to the subject for 5-30 days after the brain insult.

In certain preferred embodiments, the therapeutic combination methods described herein include administration of a liquid pharmaceutical composition described herein.

The present disclosure also includes the following enumerated items.

A1. A liquid pharmaceutical composition including topiramate or a pharmaceutically acceptable salt thereof, meglumine, and a pharmaceutically acceptable excipient.

A2. The liquid pharmaceutical composition of item A1, where the composition includes 1 mg/mL to 550 mg/mL of meglumine.

A3. The liquid pharmaceutical composition of item A1, where the composition includes 1 mg/mL to 100 mg/mL of meglumine.

A4. The liquid pharmaceutical composition of item A1, where the composition includes 1 mg/mL to 50 mg/mL of meglumine.

A5. The liquid pharmaceutical composition of item A1, where the composition includes 5 mg/mL to 50 mg/mL of meglumine.

A6. The liquid pharmaceutical composition of item A1, where the composition includes 10 mg/mL to 50 mg/mL of meglumine.

A7. The liquid pharmaceutical composition of item A1, where the composition includes 1 mg/mL to 30 mg/mL of meglumine.

A8. The liquid pharmaceutical composition of any one of items A1 to A7, where the composition includes 1 mg/mL to 100 mg/mL of topiramate or a pharmaceutically acceptable salt thereof.

A9. The liquid pharmaceutical composition of any one of items A1 to A7, where the composition includes 1 mg/mL to 65 mg/mL of topiramate or a pharmaceutically acceptable salt thereof.

A10. The liquid pharmaceutical composition of any one of items A1 to A7, where the composition includes 10 mg/mL to 70 mg/mL of topiramate or a pharmaceutically acceptable salt thereof.

A11. The liquid pharmaceutical composition of any one of items A1 to A7, where the composition includes 20 mg/mL to 65 mg/mL of topiramate or a pharmaceutically acceptable salt thereof.

A12. The liquid pharmaceutical composition of any one of items A1 to A7, where the composition includes 4 mg/mL to 30 mg/mL of topiramate or a pharmaceutically acceptable salt thereof.

A13. The liquid pharmaceutical composition of any one of items A1 to A7, where the composition includes 4 mg/mL to 20 mg/mL of topiramate or a pharmaceutically acceptable salt thereof.

A14. The liquid pharmaceutical composition of any one of items A1 to A7, where the composition includes 9 mg/mL to 11 mg/mL of topiramate or a pharmaceutically acceptable salt thereof.

A15. The liquid pharmaceutical composition of any one of items A1 to A7, where the composition includes 4 mg/mL to 6 mg/mL of topiramate or a pharmaceutically acceptable salt thereof.

A16. The liquid pharmaceutical composition of any one of items A1 to A15, where the composition further includes levetiracetam.

A17. The liquid pharmaceutical composition of item A16, where the composition includes 5 mg/mL to 500 mg/mL of levetiracetam.

A18. The liquid pharmaceutical composition of item A16, where the composition includes 5 mg/mL to 150 mg/mL of levetiracetam.

A19. The liquid pharmaceutical composition of item A16, where the composition includes 20 mg/mL to 150 mg/mL of levetiracetam.

A20. The liquid pharmaceutical composition of item A16, where the composition includes 60 mg/mL to 120 mg/mL of levetiracetam.

A21. The liquid pharmaceutical composition of item A16, where the composition includes 30 mg/mL to 60 mg/mL of levetiracetam.

A22. The liquid pharmaceutical composition of any one of items A16 to A21, where the weight ratio of levetiracetam to topiramate to is 15:1 or less.

A23. The liquid pharmaceutical composition of any one of items A16 to A21, where the weight ratio of levetiracetam to topiramate is 10:1 or less.

A24. The liquid pharmaceutical composition of any one of items A16 to A23, where the weight ratio of levetiracetam to topiramate is at least 5:1.

A25. The liquid pharmaceutical composition of any one of items A1 to A15, where the composition further includes brivaracetam.

A26. The liquid pharmaceutical composition of item A25, where the composition includes 0.5 mg/mL to 50 mg/mL of brivaracetam.

A27. The liquid pharmaceutical composition of item A25, where the composition includes 2 mg/mL to 10 mg/mL of brivaracetam.

A28. The liquid pharmaceutical composition of item A25, where the composition includes 2 mg/mL to 8 mg/mL of brivaracetam.

A29. The liquid pharmaceutical composition of item A25, where the composition includes 4 mg/mL to 10 mg/mL of brivaracetam.

A30. The liquid pharmaceutical composition of item A25, where the composition includes 4 mg/mL to 8 mg/mL of brivaracetam.

A31. The liquid pharmaceutical composition of item A25, where the composition includes 2 mg/mL to 4 mg/mL of brivaracetam.

A32. The liquid pharmaceutical composition of any one of items A25 to A31, where the weight ratio of brivaracetam to topiramate to is 1:1 or less.

A33. The liquid pharmaceutical composition of any one of items A25 to A32, where the weight ratio of brivaracetam to topiramate to is at least 1:4.

A34. The liquid pharmaceutical composition of any one of items A1 to A15, where the composition further includes padsevonil.

A35. The liquid pharmaceutical composition of item A34, where the composition includes 1 mg/mL to 100 mg/mL of padsevonil

A36. The liquid pharmaceutical composition of item A35, where the composition includes 4 mg/mL to 16 mg/mL of padsevonil.

A37. The liquid pharmaceutical composition of item A35, where the composition includes 8 mg/mL to 16 mg/mL of padsevonil.

A38. The liquid pharmaceutical composition of item A35, where the composition includes 4 mg/mL to 8 mg/mL of padsevonil.

A39. The liquid pharmaceutical composition of any one of items A34 to A38, where the weight ratio of padsevonil to topiramate is 2:1 or less.

A40. The liquid pharmaceutical composition of any one of items A34 to A38, where the weight ratio of padsevonil to topiramate is 1:2 to 2:1.

A41. The liquid pharmaceutical composition of any one of items A1 to A40, where the composition further includes atorvastatin or a pharmaceutically acceptable salt thereof.

A42. The liquid pharmaceutical composition of item A41, where the composition includes 0.1 mg/mL to 80 mg/mL atorvastatin or a pharmaceutically acceptable salt thereof.

A43. The liquid pharmaceutical composition of item A41, where the composition includes 0.1 mg/mL to 20 mg/mL atorvastatin or a pharmaceutically acceptable salt thereof.

A44. The liquid pharmaceutical composition of item A41, where the composition includes 0.1 mg/mL to 16 mg/mL atorvastatin or a pharmaceutically acceptable salt thereof.

A45. The liquid pharmaceutical composition of item A41, where the composition includes 0.2 mg/mL to 16 mg/mL atorvastatin or a pharmaceutically acceptable salt thereof.

A46. The liquid pharmaceutical composition of item A41, where the composition includes 0.4 mg/mL to 2 mg/mL atorvastatin or a pharmaceutically acceptable salt thereof.

A47. The liquid pharmaceutical composition of item A41, where the composition includes 1.2 mg/mL to 2 mg/mL atorvastatin or a pharmaceutically acceptable salt thereof.

A48. The liquid pharmaceutical composition of item A41, where the composition includes 0.6 mg/mL to 1 mg/mL atorvastatin or a pharmaceutically acceptable salt thereof.

A49. The liquid pharmaceutical composition of any one of items A41 to A48, where the weight ratio of topiramate to atorvastatin is 15:1 or less.

A50. The liquid pharmaceutical composition of any one of items A41 to A48, where the weight ratio of topiramate to atorvastatin is 10:1 or less.

A51. The liquid pharmaceutical composition of any one of items A41 to A50, where the weight ratio of topiramate to atorvastatin is at least 5:1.

A52. The liquid pharmaceutical composition of any one of items A41 to A51, where the composition includes atorvastatin sodium.

A53. The liquid pharmaceutical composition of any one of items A1 to A52, where the composition has a pH of 5.5 to 8.8.

A54. The liquid pharmaceutical composition of item A53, where the composition has a pH of 6.5 to 8.5.

A55. The liquid pharmaceutical composition of any one of items A1 to A54, where the composition further includes an acidulant.

A56. The liquid pharmaceutical composition of any one of items A1 to A55, where the acidulant is acetic acid.

A57. The liquid pharmaceutical composition of any one of items A1 to A56, where the composition further includes a calcium-scavenging agent.

A58. The liquid pharmaceutical composition of item A57, where the calcium-scavenging agent is EDTA, EGTA, BAPTA, or an alkali salt thereof.

A59. The liquid pharmaceutical composition of item A57 or A58, where the composition includes 0.001% to 5.0% (w/v) of the calcium-scavenging agent.

A60. The liquid pharmaceuticals composition of item A57 or A58, where the composition includes 0.005% to 0.5% (w/v) of the calcium-scavenging agent.

A61. The liquid pharmaceuticals composition of item A57 or A58, where the composition includes 0.01% to 0.1% (w/v) of the calcium-scavenging agent.

A62. The liquid pharmaceutical composition of any one of items A1 to A61, where the composition further includes an emulsifier.

A63. The liquid pharmaceutical composition of item A62, where the composition includes 0.001% to 5.0% (w/v) of the emulsifier.

A64. The liquid pharmaceutical composition of item A62 or A63, where the emulsifier is polyoxyethylene (20) sorbitan monooleate (polysorbate 80).

A65. The liquid pharmaceutical composition of any one of items A1 to A64, where the composition is formulated for parenteral administration.

A66. The liquid pharmaceutical composition of item A65, where the composition is formulated for intravenous administration, subcutaneous administration, or intramuscular administration.

A67. The liquid pharmaceutical composition of any one of items A1 to A66, where the composition is formulated for oral administration.

A68. The liquid pharmaceutical composition of any one of items A1 to A67, where the composition is a dosage form.

A69. The liquid pharmaceutical composition of any one of items A1 to A68, where the composition is aqueous.

A70. A kit including a first container and a second container, the first container including topiramate or a pharmaceutically acceptable salt thereof, and the second container including a pharmaceutically acceptable aqueous solution of meglumine.

A71. The kit of item A70, where topiramate or a pharmaceutically acceptable salt thereof is present in the first container in a crystalline, micronized form.

A72. The kit of item A70 or A71, where the pharmaceutically acceptable aqueous solution includes 1 mg/mL to 550 mg/mL of meglumine.

A73. The kit of any one of items A70 to A72, where topiramate or a pharmaceutically acceptable salt thereof is present in an amount sufficient to produce a composition including 1 mg/mL to 65 mg/mL of topiramate upon combination with the pharmaceutically acceptable aqueous solution of meglumine.

A74. The kit of any one of items A70 to A72, where topiramate or a pharmaceutically acceptable salt thereof is present in an amount sufficient to produce a composition including 10 mg/mL to 70 mg/mL of topiramate upon combination with the pharmaceutically acceptable aqueous solution of meglumine.

A75. The kit of any one of items A70 to A72, where topiramate or a pharmaceutically acceptable salt thereof is present in an amount sufficient to produce a composition including 20 mg/mL to 65 mg/mL of topiramate upon combination with the pharmaceutically acceptable aqueous solution of meglumine.

A76. The kit of any one of items A70 to A72, where topiramate or a pharmaceutically acceptable salt thereof is present in an amount sufficient to produce a composition including 4 mg/mL to 20 mg/mL of topiramate upon combination with the pharmaceutically acceptable aqueous solution of meglumine.

A77. The kit of any one of items A70 to A72, where topiramate or a pharmaceutically acceptable salt thereof is present in an amount sufficient to produce a composition including 9 mg/mL to 11 mg/mL of topiramate upon combination with the pharmaceutically acceptable aqueous solution of meglumine.

A78. The kit of any one of items A70 to A77, where the first container further includes levetiracetam.

A79. The kit of item A78, where levetiracetam is present in an amount sufficient to produce a composition including 5 mg/mL to 500 mg/mL of levetiracetam upon combination with the pharmaceutically acceptable aqueous solution of meglumine.

A80. The kit of item A78, where levetiracetam is present in an amount sufficient to produce a composition including 60 mg/mL to 120 mg/mL of levetiracetam of levetiracetam upon combination with the pharmaceutically acceptable aqueous solution of meglumine.

A81. The kit of item A78, where levetiracetam is present in an amount sufficient to produce a composition including 60 mg/mL to 120 mg/mL of levetiracetam of levetiracetam upon combination with the pharmaceutically acceptable aqueous solution of meglumine.

A82. The kit of item A78, where levetiracetam is present in an amount sufficient to produce a composition including 30 mg/mL to 60 mg/mL of levetiracetam upon combination with the pharmaceutically acceptable aqueous solution of meglumine.

A83. The kit of any one of items A78 to A82, where the weight ratio of levetiracetam to topiramate is 15:1 or less.

A84. The kit of any one of items A78 to A82, where the weight ratio of levetiracetam to topiramate is 10:1 or less.

A85. The kit of any one of items A78 to A84, where the weight ratio of levetiracetam to topiramate is at least 5:1.

A86. The kit of any one of items A70 to A77, where the first container further includes brivaracetam.

A87. The kit of item A86, where brivaracetam is present in an amount sufficient to produce a composition including 0.5 mg/mL to 50 mg/mL of brivaracetam upon combination with the pharmaceutically acceptable aqueous solution of meglumine.

A88. The kit of item A86, where brivaracetam is present in an amount sufficient to produce a composition including 2 mg/mL to 10 mg/mL of brivaracetam upon combination with the pharmaceutically acceptable aqueous solution of meglumine.

A89. The kit of item A86, where brivaracetam is present in an amount sufficient to produce a composition including 2 mg/mL to 8 mg/mL of brivaracetam upon combination with the pharmaceutically acceptable aqueous solution of meglumine.

A90. The kit of item A86, where brivaracetam is present in an amount sufficient to produce a composition including 4 mg/mL to 10 mg/mL of brivaracetam upon combination with the pharmaceutically acceptable aqueous solution of meglumine.

A91. The kit of item A86, where brivaracetam is present in an amount sufficient to produce a composition including 4 mg/mL to 8 mg/mL of brivaracetam upon combination with the pharmaceutically acceptable aqueous solution of meglumine.

A92. The kit of item A86, where brivaracetam is present in an amount sufficient to produce a composition including 2 mg/mL to 4 mg/mL of brivaracetam upon combination with the pharmaceutically acceptable aqueous solution of meglumine.

A93. The kit of any one of items A86 to A92, where the weight ratio of brivaracetam to topiramate to is 1:1 or less.

A94. The kit of any one of items A86 to A93, where the weight ratio of brivaracetam to topiramate to is at least 1:4.

A95. The kit of any one of items A70 to A77, where the first container further includes padsevonil.

A96. The kit of item A95, where padsevonil is present in an amount sufficient to produce a composition including 1 mg/mL to 100 mg/mL of padsevonil upon combination with the pharmaceutically acceptable aqueous solution of meglumine.

A97. The kit of item A95, where padsevonil is present in an amount sufficient to produce a composition including 4 mg/mL to 16 mg/mL of padsevonil upon combination with the pharmaceutically acceptable aqueous solution of meglumine.

A98. The kit of item A95, where padsevonil is present in an amount sufficient to produce a composition including 8 mg/mL to 100 mg/mL of padsevonil upon combination with the pharmaceutically acceptable aqueous solution of meglumine.

A99. The kit of item A95, where padsevonil is present in an amount sufficient to produce a composition including 4 mg/mL to 8 mg/mL of padsevonil upon combination with the pharmaceutically acceptable aqueous solution of meglumine.

A100. The kit of any one of items A95 to A99, where the weight ratio of padsevonil to topiramate to is 2:1 or less.

A101. The kit of any one of items A95 to A100, where the weight ratio of padsevonil to topiramate to is 2:1 or less.

A102. The kit of any one of items A95 to A99, where the weight ratio of padsevonil to topiramate to is 1:2 to 2:1.

A103. The kit of any one of items A70 to A102, where the first container further includes atorvastatin or a pharmaceutically acceptable salt thereof.

A104. The kit of item A103, where atorvastatin or a pharmaceutically acceptable salt thereof is present in an amount sufficient to produce a composition including 0.1 mg/mL to 80 mg/mL atorvastatin or a pharmaceutically acceptable salt thereof upon combination with the pharmaceutically acceptable aqueous solution of meglumine.

A105. The kit of item A103, where atorvastatin or a pharmaceutically acceptable salt thereof is present in an amount sufficient to produce a composition including 0.1 mg/mL to 20 mg/mL atorvastatin or a pharmaceutically acceptable salt thereof upon combination with the pharmaceutically acceptable aqueous solution of meglumine.

A106. The kit of item A103, where atorvastatin or a pharmaceutically acceptable salt thereof is present in an amount sufficient to produce a composition including 0.1 mg/mL to 16 mg/mL atorvastatin or a pharmaceutically acceptable salt thereof upon combination with the pharmaceutically acceptable aqueous solution of meglumine.

A107. The kit of item A103, where atorvastatin or a pharmaceutically acceptable salt thereof is present in an amount sufficient to produce a composition including 0.2 mg/mL to 16 mg/mL atorvastatin or a pharmaceutically acceptable salt thereof upon combination with the pharmaceutically acceptable aqueous solution of meglumine.

A108. The kit of item A103, where atorvastatin or a pharmaceutically acceptable salt thereof is present in an amount sufficient to produce a composition including 0.4 mg/mL to 2 mg/mL atorvastatin or a pharmaceutically acceptable salt thereof upon combination with the pharmaceutically acceptable aqueous solution of meglumine.

A109. The kit of item A103, where atorvastatin or a pharmaceutically acceptable salt thereof is present in an amount sufficient to produce a composition including 1.2 mg/mL to 2 mg/mL atorvastatin or a pharmaceutically acceptable salt thereof upon combination with the pharmaceutically acceptable aqueous solution of meglumine.

A110. The kit of item A103, where atorvastatin or a pharmaceutically acceptable salt thereof is present in an amount sufficient to produce a composition including 0.6 mg/mL to 1 mg/mL atorvastatin or a pharmaceutically acceptable salt thereof upon combination with the pharmaceutically acceptable aqueous solution of meglumine.

A111. The kit of any one of items A103 to A110, where the weight ratio of topiramate to atorvastatin is 15:1 or less.

A112. The kit of any one of items A103 to A111, where the weight ratio of topiramate to atorvastatin is 10:1 or less.

A113. The kit of any one of items A103 to A112, where the weight ratio of topiramate to atorvastatin is at least 5:1.

A114. The kit of any one of items A103 to A113, where the composition includes atorvastatin sodium.

A115. The kit of any one of items A70 to A114, where the first container contents and the second container contents upon combination produce a composition having a pH of 6.5 to 8.5.

A116. The kit of any one of items A70 to A115, where the kit further includes an acidulant.

A117. The kit of item A116, where the pharmaceutically acceptable aqueous solution of meglumine includes the acidulant.

A118. The kit of item A116 or A117, where the acidulant is acetic acid.

A119. The kit of any one of items A70 to A118, where the kit further includes a calcium-scavenging agent.

A120. The kit of item A119, where the calcium-scavenging agent is EDTA, EGTA, BAPTA, or an alkali salt thereof.

A121. The kit of item A119 or A120, where the kit further includes 0.001% to 5.0% (w/v) of the calcium-scavenging agent.

A122. The kit of item A119 or A120, where the kit further includes 0.005% to 0.5% (w/v) of the calcium-scavenging agent.

A123. The kit of item A119 or A120, where the kit further includes 0.01% to 0.1% (w/v) of the calcium-scavenging agent.

A124. The kit of any one of items A119 to A123, where the first container includes the calcium-scavenging agent.

A125. The kit of any one of items A70 to A124, where the kit further includes an emulsifier.

A126. The kit of item A125, where the pharmaceutically acceptable aqueous solution of meglumine includes the emulsifier.

A127. The kit of item A125 or A126, where the emulsifier is present in an amount sufficient to produce a composition including 0.001% to 5.0% (w/v) of the emulsifier upon combination of the first container contents and the second container contents.

A128. The kit of any one of items A125 to A127, where the emulsifier is polyoxyethylene (20) sorbitan monooleate (polysorbate 80).

A129. The kit of any one of items A70 to A128, where combination of the first container contents and the second container contents produces a composition for parenteral administration.

A130. The kit of item A129, where combination of the first container contents and the second container contents produces a composition for intravenous administration, subcutaneous administration, or intramuscular administration.

A131. The kit of any one of items A70 to A128, where combination of the first container contents and the second container contents produces a composition for oral administration.

A132. The kit of any one of items A70 to A131, where combination of the first container contents and the second container contents produces a dosage form.

A133. The kit of any one of items A70 to A132, where the first container contents are solid.

A134. The kit of item A133, where the first container contents are lyophilized.

A135. A method of treating a patient in need thereof, the method including administering to the patient a therapeutically effective amount of the composition of any one of items A1 to A69.

A136. A method of treating a patient in need thereof, the method including combining the first container contents and the second container contents in the kit of any one of items A70 to A134 to produce a liquid pharmaceutical composition, and administering a therapeutically effective amount of the liquid pharmaceutical composition to the patient.

A137. The method of item A135 or A136, where the therapeutically effective amount is an amount providing 0.5 mg/kg/day to 20 mg/kg/day of topiramate.

A138. The method of item A135 or A136, where the therapeutically effective amount is an amount providing 2 mg/kg/day to 15 mg/kg/day of topiramate.

A139. The method of any one of items A135 to A138, where the therapeutically effective amount is an amount providing at least 40 mg/day of topiramate.

A140. The method of any one of items A135 to A138, where the therapeutically effective amount is an amount providing at least 100 mg/day of topiramate.

A141. The method of any one of items A135 to A138, where the therapeutically effective amount is an amount providing at least 200 mg/day of topiramate.

A142. The method of any one of items A135 to A138, where the therapeutically effective amount is an amount providing 1200 mg/day or less of topiramate.

A143. The method of any one of items A135 to A138, where the therapeutically effective amount is an amount providing 600 mg/day or less of topiramate.

A144. The method of any one of items A135 to A138, where the therapeutically effective amount is an amount providing 400 mg/day or less of topiramate.

A145. The method of any one of items A135 to A138, where the therapeutically effective amount is an amount providing 300 mg/day or less of topiramate.

A146. The method of any one of items A135 to A144, where the therapeutically effective amount is an amount providing 2.5 mg/kg/day to 150 mg/kg/day of levetiracetam.

A147. The method of any one of items A135 to A144, where the therapeutically effective amount is an amount providing 10 mg/kg/day to 75 mg/kg/day of levetiracetam.

A148. The method of any one of items A135 to A144, where the therapeutically effective amount is an amount providing 13.75 mg/kg/day to 41 mg/kg/day of levetiracetam.

A149. The method of any one of items A135 to A144, where the therapeutically effective amount is an amount providing 0.5 mg/kg/day to 10 mg/kg/day of brivaracetam.

A150. The method of any one of items A135 to A144, where the therapeutically effective amount is an amount providing 0.5 mg/kg/day to 5 mg/kg/day of brivaracetam.

A151. The method of any one of items A135 to A150, where the therapeutically effective amount is an amount providing at least 100 mg/day of brivaracetam.

A152. The method of any one of items A135 to A151, where the therapeutically effective amount is an amount providing 400 mg/day or less of brivaracetam.

A153. The method of any one of items A135 to A151, where the therapeutically effective amount is an amount providing 300 mg/day or less of brivaracetam.

A154. The method of any one of items A135 to A144, where the therapeutically effective amount is an amount providing at least 400 mg/day of padsevonil.

A155. The method of any one of items A135 to A154, where the therapeutically effective amount is an amount providing 800 mg/day or less of padsevonil.

A156. The method of any one of items A135 to A155, where the therapeutically effective amount is an amount providing 600 mg/day or less of padsevonil.

A157. The method of any one of items A135 to A156, where the therapeutically effective amount is an amount providing 0.1 to 2.0 mg/kg/day of atorvastatin.

A158. The method of any one of items A135 to A156, where the therapeutically effective amount is an amount providing 0.2 to 1.5 mg/kg/day of atorvastatin.

A159. The method of any one of items A135 to A158, where the therapeutically effective amount is an amount providing at least 20 mg/day of atorvastatin.

A160. The method of any one of items A135 to A159, where the therapeutically effective amount is an amount providing 80 mg/day or less of atorvastatin.

A161. The method of any one of items A135 to A159, where the therapeutically effective amount is an amount providing 60 mg/day or less of atorvastatin.

A162. The method of any one of items A135 to A161, where the liquid pharmaceutical composition is administered parenterally.

A163. The method of item A162, where the liquid pharmaceutical composition is administered intravenously, subcutaneously, or intramuscularly.

A164. The method of any one of items A135 to A161, where the liquid pharmaceutical composition is administered orally.

A165. The method of any one of items A135 to A164, where the patient is in need of a treatment for a disorder or condition selected from the group consisting of epilepsy, seizures, anoxia, stroke, traumatic brain injury, brain infection, brain abscess, aneurysm, subarachnoid hemorrhage, status epilepticus, refractory status epilepticus, refractory partial onset seizures (POS), addiction (e.g., gambling addiction or drug addiction), migraines, substance dependence, alcoholism, cocaine dependence, opioid dependence, nicotine dependence, metabolic syndrome X, diabetes mellitus, type 2, vomiting, obsessive-compulsive disorder, refractory generalized social phobia, Tourette Syndrome, levodopa-induced dyskinesia in Parkinson's Disease, Prader-Willi syndrome, multiple sclerosis, Lennox-Gastaut Syndrome, Dravet's syndrome, bipolar disorder, obesity, post-traumatic stress disorder, headaches (e.g., cluster headaches or severe headaches), and conditions caused by exposure to a chemical warfare nerve agent.

A166. The method of any one of items A135 to A165, where the patient is in need of neuroprotection.

A167. The method of any one of items A135 to A165, where the patient is in need of a treatment for a disorder or condition selected from the group consisting of traumatic brain injury, stroke, a brain infection, and subarachnoid hemorrhage.

A168. The method of item A167, where the patient is in need of a treatment for traumatic brain injury.

A169. The method of item A167, where the patient is in need of a treatment for stroke.

A170. The method of item A167, where the patient is in need of a treatment for a brain infection.

A171. The method of item A170, where the patient is in need of a treatment for encephalitis, meningoencephalitis, or a brain abscess.

The present disclosure also includes the following enumerated items.

B1. A method of treating epileptogenesis in a subject after brain insult, the method including administering to the subject a therapeutically effective amount of a therapeutic combination including two to five drugs selected from the group consisting of anti-inflammatory drugs, antioxidant drugs, neuroprotective drugs, GABA-potentiating drugs, glutamate-suppressing drugs, drugs with presynaptic effects on neuronal excitability, drugs having a metabolic mode of action, and pharmaceutically acceptable salts thereof, provided that the two to five drugs are all different.

B2. The method of item B1, where each anti-inflammatory drug is independently ibuprofen, celecoxib, parecoxib, a sartan, atorvastatin, fingolimod, anakinra, or agmatine.

B3. The method of item B1, where each antioxidant drug is independently α-tocopherol, deferoxamine, N-acetylcysteine, sulforaphane, or melatonin.

B4. The method of item B1, where each neuroprotective drug is independently gabapentin, pregabalin, ifenprodil, perampanel, memantine, agmatine, celecoxib, or ceftriaxone.

B5. The method of item B1, where each GABA-potentiating drug and each glutamate-suppressing drug is independently topiramate, valproate, phenobarbital, padsevonil, deferoxamine, ceftriaxone, ifenprodil, perampanel, or memantine.

B6. The method of item B1, where each drug with presynaptic effects on neuronal excitability is independently levetiracetam, brivaracetam, etiracetam, padsevonil, gabapentin, pregabalin, or valproate.

B7. The method of item B1, where each drug having a metabolic mode of action is independently stiripentol, 2-deoxy-D-glucose, 5-azacitidine, decitabine, β-hydroxybutyrate, or vorinostat.

B8. The method of item B1, where the therapeutic combination is a combination of topiramate, levetiracetam, and deferoxamine or a pharmaceutically acceptable salt thereof.

B9. The method of item B1, where the therapeutic combination is a combination of topiramate, levetiracetam, and atorvastatin or a pharmaceutically acceptable salt thereof.

B10. The method of item B1, where the therapeutic combination is a combination of levetiracetam, ceftriaxone, and atorvastatin or a pharmaceutically acceptable salt thereof.

B11. The method of item B1, where the therapeutic combination is a combination of topiramate and levetiracetam.

B12. The method of item B1, where the fixed therapeutic combination is a combination of topiramate, levetiracetam, and ceftriaxone or a pharmaceutically acceptable salt thereof.

B13. The method of item B1, where the therapeutic combination is a combination of topiramate, levetiracetam, and gabapentin or a pharmaceutically acceptable salt thereof.

B14. The method of item B1, where the therapeutic combination is a combination of topiramate, levetiracetam, and pregabalin or a pharmaceutically acceptable salt thereof.

B15. The method of item B1, where the therapeutic combination is s a combination of levetiracetam, topiramate, and α-tocopherol.

B16. The method of item B1, where the therapeutic combination is a combination of levetiracetam, deferoxamine or a pharmaceutically acceptable salt thereof, and melatonin.

B17. The method of item B1, where the therapeutic combination is a combination of levetiracetam, deferoxamine or a pharmaceutically acceptable salt thereof, and celecoxib.

B18. The method of item B1, where the therapeutic combination is a combination of levetiracetam, deferoxamine or a pharmaceutically acceptable salt thereof, gabapentin or a pharmaceutically acceptable salt thereof, and fingolimod or a pharmaceutically acceptable salt thereof.

B19. The method of item B1, where the therapeutic combination is a combination of levetiracetam, atorvastatin or a pharmaceutically acceptable salt thereof, and ceftriaxone.

B20. The method of item B1, where the therapeutic combination is a combination of levetiracetam and perampanel or a pharmaceutically acceptable salt thereof.

B21. The method of item B1, where the therapeutic combination is a combination of levetiracetam, perampanel or a pharmaceutically acceptable salt thereof, and ceftriaxone.

B22. The method of item B1, where the therapeutic combination is a combination of levetiracetam, parecoxib, and anakinra.

B23. The method of item B1, where the therapeutic combination is a combination of levetiracetam and phenobarbital.

B24. The method of item B1, where the therapeutic combination is a combination of brivaracetam, and topiramate.

B25. The method of item B1, where the therapeutic combination is a combination of brivaracetam, topiramate, and ceftriaxone.

B26. The method of item B1, where the therapeutic combination is a combination of brivaracetam, ceftriaxone, and atorvastatin

B27. The method of item B1, where the therapeutic combination is a combination of brivaracetam, ceftriaxone, and atorvastatin

B28. The method of item B1, where the therapeutic combination is a combination of brivaracetam and perampanel.

B29. The method of item B1, where the therapeutic combination is a combination of brivaracetam, perampanel, and ceftriaxone.

B30. The method of item B1, where the therapeutic combination is a combination of valproate or a pharmaceutically acceptable salt thereof, losartan or a pharmaceutically acceptable salt thereof, and memantine or a pharmaceutically acceptable salt thereof.

B31. The method of any one of items B1 to B30, where the therapeutic combination includes three of the drugs.

B32. The method of any one of items B1 to B31, where the dose of each drug in the combination is the highest tolerable dose in the individual patient, when the drug is administered in the therapeutic combination.

B33. The method of any one of items B1 to B31, where the therapeutically effective dose represents 30% to 75% of the highest approved dose for administration to a human as a monotherapy.

B34. The method of any one of items B1 to B31, where the therapeutically effective dose represents 50% to 75% of the highest approved dose for administration to a human as a monotherapy.

B35. The method of item B1, where agmatine is administered at a dose of 0.89-3.56 g/day.

B36. The method of item B1, where agmatine is administered at a dose of 0.89-2.67 g/day.

B37. The method of item B1, where agmatine is administered at a dose of 1.78-2.67 g/day.

B38. The method of item B1, where anakinra is administered at a dose of 2-8 mg/kg/day.

B39. The method of item B1, where anakinra is administered at a dose of 2-6 mg/kg/day.

B40. The method of item B1, where anakinra is administered at a dose of 4-6 mg/kg/day.

B41. The method of item B1, where brivaracetam is administered at a dose of 100-400 mg/day.

B42. The method of item B1, where brivaracetam is administered at a dose of 100-300 mg/day.

B43. The method of item B1, where brivaracetam is administered at a dose of 200-300 mg/day.

B44. The method of item B1, where padsevonil is administered at a dose of 200-800 mg/day.

B45. The method of item B1, where padsevonil is administered at a dose of 200-600 mg/day.

B46. The method of item B1, where padsevonil is administered at a dose of 400 mg/day.

B47. The method of item B1, where ceftriaxone is administered at a dose of 1-4 g/day.

B48. The method of item B1, where ceftriaxone is administered at a dose of 1-3 g/day.

B49. The method of item B1, where ceftriaxone is administered at a dose of 2-3 g/day.

B50. The method of item B1, where deferoxamine is administered at a dose of 15.5-62 mg/kg/day.

B51. The method of item B1, where deferoxamine is administered at a dose of 15.5-46.5 mg/kg/day.

B52. The method of item B1, B50, or B51, where deferoxamine is administered at a dose of up to a maximum of 6000 mg/day.

B53. The method of item B1, B50, B51, or B52, where deferoxamine is administered at a dose of up to a maximum of 4500 mg/day.

B54. The method of item B1, where fingolimod is administered at a dose of 0.31-1.25 mg/day.

B55. The method of item B1, where fingolimod is administered at a dose of 0.31-0.94 mg/day.

B56. The method of item B1, where fingolimod is administered at a dose of 0.625-0.94 mg/day.

B57. The method of item B1, where gabapentin is administered at a dose of 800-3200 mg/day.

B58. The method of item B1, where gabapentin is administered at a dose of 800-2400 mg/day.

B59. The method of item B1, where gabapentin is administered at a dose of 1600-2400 mg/day.

B60. The method of item B1, where ifenprodil is administered at a dose of 10-40 mg/day.

B61. The method of item B1, where ifenprodil is administered at a dose of 10-30 mg/day.

B62. The method of item B1, where ifenprodil is administered at a dose of 20-30 mg/day.

B63. The method of item B1, where levetiracetam is administered at a dose of 13.75-55 mg/kg/day.

B64. The method of item B1, where levetiracetam is administered at a dose of 13.75-41 mg/kg/day

B65. The method of item B1, where levetiracetam is administered at a dose of 27.5-41 mg/kg/day

B66. The method of item B1, B63, B64, or B65, where levetiracetam is administered at a dose of 6000 mg/day or less.

B67. The method of item B1, B63, B64, or B65, where levetiracetam is administered at a dose of 4500 mg/day or less.

B68. The method of item B1, B63, B64, or B65, where levetiracetam is administered at a dose of 3000 mg/day or less.

B69. The method of item B1, B63, B64, B65, B66, B67, or B68, where levetiracetam is administered at a dose of at least 900 mg/day.

B70. The method of item B1, B63, B64, B65, B66, B67, or B68, where levetiracetam is administered at a dose of at least 3000 mg/day.

B71. The method of item B1, where losartan is administered at a dose of 25-100 mg/day.

B72. The method of item B1, where losartan is administered at a dose of 25-75 mg/day.

B73. The method of item B1, where losartan is administered at a dose of 50-75 mg/day.

B74. The method of item B1, where melatonin is administered at a dose of 5-20 mg/day.

B75. The method of item B1, where melatonin is administered at a dose of 5-15 mg/day.

B76. The method of item B1, where melatonin is administered at a dose of 10-15 mg/day.

B77. The method of item B1, where memantine is administered at a dose of 7-28 mg/day.

B78. The method of item B1, where memantine is administered at a dose of 7-21 mg/day.

B79. The method of item B1, where memantine is administered at a dose of 14-21 mg/day.

B80. The method of item B1, where N-acetylcysteine is administered at a dose of 75-300 mg/kg/day.

B81. The method of item B1, where N-acetylcysteine is administered at a dose of 75-225 mg/kg/day.

B82. The method of item B1, where N-acetylcysteine is administered at a dose of 150-225 mg/kg/day.

B83. The method of item B1, where perampanel is administered at a dose of 6-24 mg/day.

B84. The method of item B1, where perampanel is administered at a dose of 6-18 mg/day.

B85. The method of item B1, where perampanel is administered at a dose of 12-18 mg/day.

B86. The method of item B1, where phenobarbital is administered at a dose of 50-200 mg.

B87. The method of item B1, where phenobarbital is administered at a dose of 50-150 mg/day.

B88. The method of item B1, where phenobarbital is administered at a dose of 100-150 mg/day.

B89. The method of item B1, where sulforaphane is administered at a dose of 15-60 mg/day.

B90. The method of item B1, where sulforaphane is administered at a dose of 15-45 mg/day.

B91. The method of item B1, where sulforaphane is administered at a dose of 30-45 mg/day.

B92. The method of item B1, where topiramate is administered at dose of 100-400 mg/day.

B93. The method of item B1, where topiramate is administered at a dose of 100-300 mg/day.

B94. The method of item B1, where topiramate is administered at a dose of 200-300 mg/day.

B95. The method of item B1, where valproate is administered at a dose of 750-3000 mg/day.

B96. The method of item B1, where valproate is administered at a dose of 750-2250 mg/day.

B97. The method of item B1, where valproate is administered at a dose of 1500-2250 mg/day.

B98. The method of item B1, where α-tocopherol is administered at a dose of 3.75-15 mg/day.

B99. The method of item B1, where α-tocopherol is administered at a dose of 3.75-11.25 mg/day.

B100 The method of item B1, where α-tocopherol is administered at a dose of 7.5-11.25 mg/day.

B101. The method of any one of items B1 to B34, where the therapeutic combination is administered as the liquid pharmaceutical composition of any one of items A1 to A69.

B102. The method of any one of items B1 to B101, where the therapeutic combination has a tolerability and/or efficacy that are greater than the expected additive tolerability and/or efficacy of the drugs in the therapeutic combination.

B103. The method of any one of items B1 to B102, where the therapeutic combination is initially administered to the subject intravenously for 1 to 30 days.

B104. The method of any one of items B1 to B103, where the therapeutic combination is administered to the subject intramuscularly, subcutaneously, orally, percutaneously, sublingually, buccally, intranasally, by inhalation, or rectally.

B105. The method of any one of items B1 to B104, where, after the therapeutic combination administration is initiated, the therapeutic combination administration is maintained by prolonged oral or parenteral administration.

B106. The method of item B105, where the therapeutic combination administration is maintained for 3 to 6 months following the brain insult.

B107. The method of any one of items B1 to B106, where at least two drugs are present in the same pharmaceutical composition.

B108. The method of any one of items B1 to B106, where at least three drugs are present in the same pharmaceutical composition.

B109. The method of any one of items B1 to B109, where the administering step is initiated within 7 days after the brain insult.

B110. The method of item B109, where the administering step is initiated within 48 h after the brain insult.

B111. The method of item B109, where the administering step is initiated within 24 h after the brain insult.

B112. The method of item B109, where the administering step is initiated within 8 h after the brain insult.

B113. The method of any one of items B1 to B112, where the therapeutic combination is administered to the subject for a period of 3 day to 3 months after the insult.

B114. The method of item B113, where the therapeutic combination is administered to the subject for 5-30 days after the brain insult.

### Definitions

The term “α-amino-3-hydroxy-5-methyl-4-isoxazolepropionic acid (AMPA) receptor antagonist,” as used herein, refers to a compound that binds to and blocks the activation of α-amino-3-hydroxy-5-methyl-4-isoxazolepropionic acid (AMPA) receptor. Non-limiting examples of AMPA receptor antagonists include perampanel, Irampanel (Dimethyl-(2-[2-(3-phenyl-[1,2,4]oxadiazol5-yl)-phenoxyl]-ethyl)-amine hydrochloride), and talampanel. AMPA antagonists are characterized in that they reduce/block the glutamate induced currents as evaluated by whole cell voltage clamp experiments in cells (cell lines or primary neuronal cells) expressing the AMPA receptor. Compounds with an IC50 of below 5 μM are exemplary selective AMPA antagonists. For perampanel, which represents a non-competitive selective allosteric modulator of the AMPA receptor, IC50 is in the range of 0.56 μM.

The term “anti-inflammatory drug,” as used herein, refers to a compound selected from the group consisting of cyclooxygenase (COX) inhibitors; HMGCR inhibitors; selective angiotensin II receptor antagonists (sartans); sphingosine-1-phosphate receptor 1 (S1P1-receptor) modulators; interleukin-1-receptor antagonists; and agmatine.

The term “antioxidant drug,” as used herein, refers to α-tocopherol, deferoxamine, N-acetylcysteine, sulforaphane, or melatonin.

The term “COX inhibitor,” as used herein, refers to a compound that inhibits cyclooxygenase (COX) enzymes. The COX inhibitor may inhibit cyclooxygenase 1 (COX1), cyclooxygenase 2 (COX2), or both. The COX inhibitor may be a selective COX inhibitor (e.g., a selective COX2 inhibitor). COX inhibitors that bind to COX2 with an affinity that is at least 10-fold greater than for COX1 are selective COX2 inhibitors. Non-limiting examples of COX inhibitors include ibuprofen, flurbiprofen, naproxen, ketoprofen, tiaprofenic acid, diclofenac, indomethacin, acemetacin, flufenamic acid, mefenamic acid, piroxicam, tenoxicam, meloxicam, lornoxicam, acetyl salicylic acid, celecoxib, parecoxib, etoricoxib, rofecoxib, valdecoxib, and lumiracoxib. COX inhibitors are defined as compounds which are capable of inhibiting human cyclooxygenase activity with an IC50 concentration of 10 μM or lower. Selective COX inhibitors demonstrate an at least 10 fold lower IC50 for inhibition of one COX enzyme subtype (e.g. the COX2) over the other (e.g. COX1) subtype enzyme. Multiple COX inhibition assays are described which are all applicable to determine COX inhibition. One example is the use of cell-free enzyme preparations using clonally expressed COX enzyme preparations, evaluating the inhibition of the peroxidase activity of COX enzymes.

The term “drugs having a metabolic mode of action,” as used herein, refers to a compound selected from the group consisting of stiripentol, 2-deoxy-D-glucose, 5-azacitidine, decitabine, β-hydroxybutyrate, and vorinostat.

The term “drug with presynaptic effects on neuronal excitability,” as used herein, refers to a compound selected from the group consisting of levetiracetam, brivaracetam, padsevonil, seletracetam, known modulators of the synaptic vesicle protein 2, and modulators of presynaptic calcium channels.

The terms “GABA-potentiating drugs” and “glutamate suppressing drugs,” as used herein, refer to topiramate, valproate, phenobarbital, padsevonil, deferoxamine, ceftriaxone, ifenprodil, α-amino-3-hydroxy-5-methyl-4-isoxazolepropionic acid (AMPA) receptor antagonist, or N-methyl-D-aspartate receptor (NMDA) antagonist.

The term “HMGCR inhibitor,” as used herein, refers to a compound that inhibits 3-hydroxy-3-methyl-glutaryl-coenzyme A reductase. Non-limiting examples of HMGCR inhibitors include atorvastatin, cerivastatin, fluvastatin, lovastatin, pitavastatin, pravastatin, rosuvastatin, and simvastatin. Selective inhibitors block the catalytic domain of the HMBCR domain with an IC50 of at least 1 μM, as assessed using human purified hydroxymethylglutaryl coenzyme A reductase catalytic domain. The inhibition of enzyme activity can be also evaluated in rat and human hepatic microsomes, as they are rich in HMGCR.

The term “interleukin-1-receptor antagonist,” as used herein, refers to a compound that binds to interleukin-1-receptor. Non-limiting examples of interleukin-1-receptor antagonists include anakinra, which is a recombinant and slightly modified version of the human interleukin 1 receptor antagonist protein. Other agents are rilonacept, canakinumab, and gevokizumab, all representing biological drugs (fusion proteins or monoclonal antibodies) binding to the IL1 receptor without activating the receptor. The affinity of such biologic drugs is expected to be in the low nanomolar or picomolar range, as measured by surface plasmon resonance assay using commercial assays.

The term “modulator of presynaptic calcium channels,” as used herein, refers to a compound that binds to a presynaptic calcium channel. Non-limiting examples of modulators of presynaptic calcium channels include gabapentin, pregabalin, and valproate. Other presynapitc calcium channel blockers bind to the alpha2-delta1 or alpha2-delta2 subunit of the presynaptic calcium channel with affinities of less than 1 μM as determined by receptor binding assays using for the [³H]-pregabalin binding as competitive ligand.

The term “modulator of the synaptic vesicle protein 2,” as used herein, refers to a compound that binds to synaptic vesicle protein 2. Non-limiting examples of SV2 modulators including SV2A modulators include levetiracetam, brivaracetam, etiracetam, seletracetam, and padsevonil. SV2 modulators bind to purified SV2A protein preparations or to clonally expressed SV2A protein with an affinity of at 1 μM or lower (IC50), using [³H]-levetiracetam as competitive ligand.

The term “neuroprotective drugs or drugs increasing regeneration and plasticity,” as used herein, refers to gabapentin, pregabalin, ifenprodil, perampanel, memantine, agmatine, celecoxib, or ceftriaxone.

The term “N-methyl-D-aspartate receptor (NMDA) antagonist,” as used herein, refers to a compound that binds to and blocks the activity of N-methyl-D-aspartate receptor (NMDA). Non-limiting examples of NMDA receptor antagonists include memantine ifenprodil, ketamine, dizocilpine and dextromethorphan. NMDA antagonists are characterized in that they reduce/block the N-methyl-D-aspartate induced currents as evaluated by whole cell voltage clamp experiments in cells (cell lines or primary neuronal cells) expressing the NMDA receptor. Compounds with an IC50 of below 10 μM are selective NMDA antagonists. Both, competitive and non-competitive NMDA antagonists are included.

The term “liquid pharmaceutical composition,” as used herein, refers to those pharmaceutical compositions in the form of pharmaceutical solutions and pharmaceutical suspensions. Preferably, a liquid pharmaceutical composition (e.g., a parenteral, liquid pharmaceutical composition) is a solution.

The term “pharmaceutically acceptable,” as used herein, refers to those compounds, materials, compositions, and/or dosage forms, which are suitable for contact with the tissues of an individual (e.g., a human), without excessive toxicity, irritation, allergic response and other problem complications commensurate with a reasonable benefit/risk ratio.

The term “pharmaceutical composition,” as used herein, represents a composition containing one or more compounds described herein, formulated with a pharmaceutically acceptable excipient, and typically manufactured or sold with the approval of a governmental regulatory agency as part of a therapeutic regimen for the treatment of a disease in a mammal. Pharmaceutical compositions can be formulated, for example, for oral administration in unit dosage form (e.g., an oral solution or, alternatively, a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable salt,” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared, e.g., in situ during the final isolation and purification of the compounds described herein or separately by reacting the free base group with a suitable organic acid. Representative acid addition salts include, e.g., acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include, e.g., sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. One of skill in the art will recognize that any mention of a drug compound includes within its scope the pharmaceutically acceptable salts of the indicated drug compound.

The term “polysorbate,” as used herein, refers to oleate esters of sorbitol and its anhydrides, typically copolymerized with ethylene oxide. A preferred polysorbate is polysorbate 80 (poly(ethylene oxide) (80) sorbitan monolaurate).

The term “prolonged,” as used herein in relation to administration of a therapeutic combination, refers to a chronic administration of a therapeutic combination for a period of at least 3 months.

The term “selective angiotensin II receptor antagonist,” as used herein, refers to a compound that binds to and blocks the activation of angiotensin II receptor type 1, known as AT₁ receptors. Selective angiotensin II receptor antagonists are also known as sartans. Non-limiting examples of sartans include losartan, valsartan, irbesartan, telmisartan, candesartan, olmesartan, and azilsartan. Sartans bind to AT₁ receptors with the affinity that is at least 10-fold greater than for AT₂ receptors. Respective compounds are evaluated for their in vitro AT receptor binding affinity in the competitive inhibition of radioactively labelled [¹²⁵I]-Angiotensin II binding to the AT₁ and AT₂ receptors by a conventional ligand binding assays. AT antagonists present with affinities below 1 μM expressed as IC50 values, which are the concentrations of a compound that inhibit [¹²⁵I]-angiotensin II binding to the receptor by 50%.

The term “S1P1-receptor antagonist,” as used herein, refers to a compound that binds to sphingosine-1-phosphate receptor 1. By binding to the S1P receptors, the receptors break down with time and become inactivated, representing the mode of action of these agents. Non-limiting examples of S1P1 antagonists include fingolimod, ozanimod, ponesimod, and laquinimod. Respective S1P1 antagonists bind to the receptor with at sub-micromolar concentrations, as determined by GTP-GammaS assay resulting in an EC50 of <0.1 μM).

“Treatment” and “treating,” as used herein, refer to the medical management of a subject with the intent to improve, ameliorate, stabilize, prevent or cure a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy). “Treatment” and “treating,” as used herein, also refers to disease modification, meaning, that the expression of the disease (e.g., post-traumatic epilepsy and post-traumatic epilepsy associated central nervous system symptoms), is modified towards a less severe expression of the symptoms, including less severe or less frequent occurrence of seizures, and/or less severe or complete suppression of disease associated central nervous system symptoms such as anxiety disorders, depressive disorders, cognitive impairment including learning and memory deficits, and sleeping disorders, known to be linked with epilepsy and seizure disorders. Non-limiting examples of diseases, disorders, and conditions that may be treated using compositions, kits, and methods disclosed herein include, e.g., epilepsy, seizures, anoxia, stroke, traumatic brain injury, brain infection, brain abscess, aneurysm, subarachnoid hemorrhage, status epilepticus, refractory status epilepticus, refractory partial onset seizures (POS), addiction (e.g., gambling addiction or drug addiction), migraines, substance dependence, alcoholism, cocaine dependence, opioid dependence, nicotine dependence, metabolic syndrome X, diabetes mellitus, type 2, vomiting, obsessive-compulsive disorder, refractory generalized social phobia, Tourette Syndrome, levodopa-induced dyskinesia in Parkinson's Disease, Prader-Willi syndrome, multiple sclerosis, Lennox-Gastaut Syndrome, Dravet's syndrome, bipolar disorder, obesity, post-traumatic stress disorder, headaches (e.g., cluster headaches or severe headaches), and conditions caused by exposure to a chemical warfare nerve agent.

Preferably, a disease, disorder, or condition is post-traumatic epilepsy and post-traumatic epilepsy associated central nervous system symptoms, epilepsy, subarachnoid hemorrhage, seizures, anoxia and anoxia induced brain injury, stroke, traumatic brain injury, brain infection, brain abscess, status epilepticus, refractory status epilepticus, or refractory partial onset seizures.

### BRIEF DESCRIPTION OF THE DRAWINGS

The FIG. is a graph showing the dependence of the dependence of the topiramate aqueous solubility (mg/mL) on the concentration (mg/mL) of meglumine.

### DETAILED DESCRIPTION

In general, the invention provides compositions and methods of their use, e.g., therapeutic combination therapies. The compositions and methods described herein may be used in the treatment of a subject in need thereof, e.g., a subject suffering from epileptogenesis or epilepsy.

Therapeutic Combinations

The invention provides a therapeutic combination including two to five (e.g., two, three, four, or five) drugs selected from the group consisting of anti-inflammatory drugs, antioxidant drugs, neuroprotective drugs, GABA-potentiating drugs, glutamate-suppressing drugs, drugs with presynaptic effects on neuronal excitability, drugs having a metabolic mode of action, and pharmaceutically acceptable salts thereof. The therapeutic combinations described herein are useful in the treatment of epileptogenesis in a subject having brain insult (e.g., traumatic brain injury (TBI), stroke, or brain infection).

Advantageously, the therapeutic combinations described herein may reduce the expression of epileptic seizures in a subject (e.g., reduction of the seizure frequency and/or seizure severity), as well as disease-associated symptoms. The therapeutic combinations described herein may suppress epileptogenesis in a subject.

The following drugs may be used as therapeutic combinations:
a) anti-inflammatory drugs, e.g., a cyclooxygenase (COX) inhibitor; HMGCR inhibitor; sartan; sphingosine-1-phosphate receptor 1 (S1P1-receptor) modulator; interleukin-1-receptor antagonist; or agmatine;
b) antioxidant drugs, e.g., α-tocopherol, deferoxamine, N-acetylcysteine, sulforaphane, or melatonin;
c) neuroprotective drugs or drugs increasing regeneration and plasticity, e.g., gabapentin, pregabalin, ifenprodil, perampanel, memantine, agmatine, celecoxib, or ceftriaxone;
d) GABA-potentiating drugs and glutamate suppressing drugs, e.g., topiramate, valproate, phenobarbital, padsevonil, deferoxamine, ceftriaxone, ifenprodil, α-amino-3-hydroxy-5-methyl-4-isoxazolepropionic acid (AMPA) receptor antagonist, or N-methyl-D-aspartate receptor (NMDA) antagonist;
e) drugs with presynaptic effects on neuronal excitability, e.g., levetiracetam, a known modulator of the synaptic vesicle protein 2, or a modulator of presynaptic calcium channels; and
f) drugs having a metabolic mode of action, e.g., stiripentol, 2-deoxy-D-glucose, 5-azacitidine, decitabine, β-hydroxybutyrate, or vorinostat.

Therapeutic combinations described herein are useful for treating a brain insult, such as traumatic brain injury, e.g., open or closed brain injury, brain contusion, a brain injury as a result of brain surgery, a hypoxic brain injury following a thrombotic or hemorrhagic stroke, or a brain injury resulting from infection of the brain or resulting from a brain tumor.

Preferably, a therapeutic combination includes topiramate and levetiracetam. A particularly preferred therapeutic combination is a combination of topiramate, levetiracetam, and a third drug. Preferably, the third drug is an anti-inflammatory drug (e.g., an anti-inflammatory drug targeting the early inflammatory components of epileptogenesis), e.g., atorvastatin, celecoxib, or deferoxamine. Another preferred third drug is gabapentin or pregabalin; each of these drugs add the modulation of the pre-synaptic excitability to topiramate and levetiracetam.

Alternative preferred therapeutic combinations are (i) a combination of levetiracetam and α-tocopherol; (ii) a combination of levetiracetam, deferoxamine, and melatonin; or (iii) a combination of levetiracetam, deferoxamine, and celecoxib.

Further preferred therapeutic combinations include combinations of (i) levetiracetam, deferoxamine, gabapentin, and fingolimod; (ii) levetiracetam, atorvastatin, and ceftriaxone; (iii) levetiracetam and perampanel; (iv) levetiracetam, parecoxib, and anakinra; or (iv) levetiracetam and phenobarbital.

Yet further preferred therapeutic combinations include combinations of (i) brivaracetam and topiramate; (ii) brivaracetam and perampanel; or (iii) valproate, losartan, and memantine.

An important aspect of the combination treatment is proper timing of the treatment. The treatment with therapeutically effective doses is preferably initiated within one week after the brain insult, more preferably within 48 h after the brain insult, even more preferably within 24 h after the brain insult, and most preferably within 8 h after the brain insult. The early treatment initiation may be important for the effectiveness of the epileptogenesis treatment.

Treatment is preferably continued after initiation for a predetermined period of time, even if no clinical signs of epileptogenesis are observed during the latent period of epileptogenesis. At present, there are no known predictive markers of epileptogenesis useful for determining the initial treatment duration. Therefore, it is important to initiate the treatment early and to continue for a sufficiently long period of time. The preferred initial treatment duration is at least 3 days-long (e.g., up to 3 months-long) following the initial brain insult; more preferably, the initial treatment duration is 5-30 days. The treatment duration may be adjusted to the individual severity or the condition of the individual patient, and/or to the duration of the latency period for epileptogenesis as expected for the respective brain insult. In cases where clinical experience shows that the latent period can be very long, the initial epileptogenesis treatment may be extended beyond the initial 30-day treatment, or may lead to a long-term maintenance treatment, either with the same therapeutic combination, or with a different therapeutic combination, where the drug targeting early aspects of epileptogenesis after brain insult, e.g., early inflammation, is removed from or replaced in the therapeutic combination. Thus, the initiation epileptogenesis treatment and its consequences may be continued with a different therapeutic combination to further the epileptogenesis treatment.

The route of administration for the initial treatment (also referred to as the initiation treatment) may be selected to ensure adequate exposure and may be adjusted to the condition of the patient. Preferably, a therapeutic combination is administered intravenously. Advantageously, intravenous administration may provide rapid and/or full bioavailability of the therapeutic agent(s). Intravenous administration is also advantageous because it may be used for administration to out-patient subjects (e.g., by ambulance personnel, in the emergency room, intensive care units), subjects with reduced level of consciousness, or in subjects with disturbed enteral motility and enteral absorption. The intravenous administration can be performed as bolus injection or as infusion. The dosing frequency of infusion may be guided by the pharmacokinetics of the therapeutic combination being administered. The intravenous administration may be performed once daily, twice daily, three times daily administration, or prolonged infusion with or without a loading bolus administration. While the individual drugs can be administered separately, the preferred administration is in a therapeutic combination as a single pharmaceutical composition. Alternative administration routes include oral dosing (e.g., using an esophageal or gastric tube) and parenteral (e.g., subcutaneous, intramuscular) dosing. Alternative, non-limiting examples of dosing routes include rectal, nasal, inhalation, sublingual, buccal, and transdermal administration.

Dosing for each drug in a therapeutic combination may be selected as needed. Two separate strategies may be employed for the dose selection. The high-dose strategy aims at administering the highest tolerated dose (or highest approved dose) of each of the combination partners in the therapeutic combination. This high-dose approach provides the maximal pharmacodynamic effect for each drug. This high-dose approach is particularly appropriate for the initial treatment period (initiation treatment) immediately after the insult for up to 30 days. This approach is particularly useful for subjects with a severe brain insult and high likelihood of the epileptogenesis due to the severity or nature of the brain insult, as well as for subjects treated in the emergency room and in the intensive care unit.

Advantageously and unexpectedly, the tolerability of the preferred combinations may be comparable to the tolerability of the individual components. Without wishing to be bound by theory, the use of drugs targeting different aspects of epileptogenesis may result in the lack of the drug-specific adverse effect potentiation.

In the methods disclosed herein, treatment with the highest tolerated doses is typically not required in most subjects but may be useful in some instances. Typically, because of the combination of the different drugs, a therapeutic effect can be also achieved with a low-dose approach. The low-dose approach typically involves administration of 75% or less (e.g., 40-60%) of the highest approved dose of each drug in the therapeutic combination.

As the preferred administration route for the initiation treatment is the intravenous route, any such doses, both, for the high dose and the low dose approach, may require a loading dose at the beginning of the intravenous administration, to rapidly provide a therapeutically effective plasma and tissue levels of the active(s). Such loading doses can exceed the highest approved doses for a drug by a factor of 2-5 fold.

Typically, the loading doses may be selected according to the pharmacokinetics of each respective drug used in any given therapeutic combination. Furthermore, an approved high dose may be exceeded by up to 100% for the initiation treatment, dependent on factors for each individual subject, e.g., severity of the brain insult, general health status, other concomitant diseases, body weight, and metabolic state.

For the high dose approach, the following doses may be appropriate to represent exemplary high doses: agmatine, 3.56 g/day; anakinra, 8 mg/kg/day; atorvastatin 80 mg/day; brivaracetam, 400 mg/day; ceftriaxone, 4 g/day; deferoxamine, 62 mg/kg/day up to a maximum of 6000 mg/day; fingolimod, 1.25 mg/day; gabapentin, 3,200 mg/day; ifenprodil, 40 mg/day; levetiracetam, 55 mg/kg/day up to a maximum of 6,000 mg/day; losartan, 100 mg/day; melatonin, 20 mg/day; memantine, 28 mg/day; N-acetylcysteine, 300 mg/kg/day; padsevonil, 800 mg/day; perampanel, 24 mg/day; phenobarbital, 200 mg/day; sulforaphane, 60 mg/day; topiramate, 400-600 mg/day; valproate, 3,000 mg/day; α-tocopherol, 15 mg/day; seletracetam, 100-160 mg/day.

For the low dose approach for initiation treatment, the therapeutically effective doses for each drug in the therapeutic combination may be 25-75% of the highest approved dose for administration as a monotherapy. For example, the low doses are as follows: agmatine, 0.89-2.67 g/day; anakinra, 2-6 mg/kg/day; atorvastatin 20-60 mg/day; brivaracetam, 100-300 mg/day; ceftriaxone, 1.0-3 g/day; deferoxamine, 15.5-46.5 mg/kg/day up to a maximal dose of 4500 mg/day; fingolimod, 0.31-0.94 mg/day; gabapentin, 800-2400 mg/day; ifenprodil, 10-30 mg/day; levetiracetam, 13.75-41 mg/kg/day up to a maximum of 3000-4500 mg/day; losartan 25-75 mg/day; melatonin, 5-15 mg/day; memantine 7-21 mg/day; N-acetylcysteine, 75-225 mg/kg/day; padsevonil, 200-600 mg/day; perampanel, 6-18 mg/day; phenobarbital, 50-150 mg/day; seletracetam, 25-75 mg/day; sulforaphane, 15-45 mg/day; topiramate, 100-300 mg/day; valproate, 750-2250 mg/day; α-tocopherol, 3.75-11.25 mg/day.

The preferred route of administration for the initiation treatment is the intravenous route, either as a single pharmaceutical composition containing the therapeutic combination or as sequence of pharmaceutical compositions containing one or more therapeutic combination drugs (e.g., as bolus or as infusion). Depending on the condition of the subject, or based on the disease state and ability of the subject, or based on availability of formulations, other routes of administration may be appropriate. For example, the treatment may be initiated as an intravenous or parenteral (e.g., intramuscular or subcutaneous) administration, and as the compliance and ability of the subject improves, the route of administration may be switched to the oral route, while remaining on the initiation treatment regimen. Routes of administration for the drugs of the therapeutic combination may be same or different. For example, one agent may be administered intravenously, and another drug may be administered subcutaneously, intramuscularly, or orally.

An important embodiment is the initiation treatment after the brain insult, which is performed during the latent period of epileptogenesis. Typically, the initiation treatment may last for up to 30 days. As the latent period may last more than 30 days, depending on the individual brain insult and the condition of the subject, the initiation treatment should be followed by a maintenance treatment. Preferably, the maintenance treatment includes oral administration for the orally bioavailable drugs. The maintenance treatment can be performed with the same therapeutic combination as in the initiation treatment, or with a different therapeutic combination, in which the drugs targeting early aspects of epileptogenesis (e.g., inflammatory processes) are discontinued from the therapeutic combination. The maintenance treatment can continue after the initiation treatment, e.g., for 3 to 6 months or chronically (e.g., to treat the remaining symptoms of epileptogenesis).

Administration Regimens and Pharmaceutical Formulations

The compounds used in the methods described herein are preferably formulated into pharmaceutical compositions for administration to human subjects in a biologically compatible form suitable for administration in vivo. Pharmaceutical compositions typically include a compound, or, for combination treatments as described herein, more than one compound in a fixed ratio, and a pharmaceutically acceptable excipient.

The individual compounds described herein can also be used in the form of the free base, in the form of salts, zwitterions, solvates, or as prodrugs, or pharmaceutical compositions thereof. All forms are within the scope of the invention. The compounds, salts, zwitterions, solvates, prodrugs, or pharmaceutical compositions thereof, may be administered to a patient in a variety of forms depending on the selected route of administration, as will be understood by those skilled in the art. The different compounds to be administered in combination can be administered as fixed combination, where two or more compounds are formulated in one dosing form, or can be administered as individual formulated compounds, but administered as combination treatment to the patient. If more than two compounds are to be administered for the combination treatment, some or all of the compounds can be administered in one fixed combination, while the remaining compounds, if any, to be administered, can be administered as individual formulated compounds or (if e.g. 4 compounds are to be combined, as two fixed combinations with to compounds each, or as a fixed combination of 3 compounds and one individual compound, or as a fixed combination of 4 compounds, or as 4 individual formulated compounds. The compounds and/or fixed combinations of compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound or any combination of compounds described herein can be administered alone or in admixture with a pharmaceutical carrier selected regarding the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of a compound into preparations which can be used pharmaceutically.

This invention also includes pharmaceutical compositions which can contain one or more pharmaceutically acceptable carriers. In making the pharmaceutical compositions, the active ingredient is typically mixed with an excipient, diluted by an excipient or enclosed within such a carrier in the form of, for example, a capsule, sachet, paper, or other container. In the case of liquid formulations e.g. for parenteral administration, such container may represent a glass or plastic vial or bottle, ampoule or other pharmaceutically acceptable container for liquid drugs. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

Dosages

The dosage of the individual compounds administered as combination treatments used in the methods described herein, or pharmaceutically acceptable salts or prodrugs thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the individual to be treated. One of skill in the art can determine the appropriate dosage based on the above factors. The compounds used in the methods described herein may be administered initially in a suitable dosage that may be adjusted as required, depending on the clinical response. In general, a suitable daily dose of each of the compounds in combinations will be that amount of each compound that is the lowest dose effective to produce a therapeutic effect. Such an effective dose will generally depend upon the factors described above.

Each of the compounds may be, for example, administered to the patient in a single dose or in multiple doses. For any such combinations, the frequency of dosing may be the same for each of the compounds to be combined or may be individually selected for each of the individual compounds to be combined. When multiple doses are administered, the doses may be separated from one another by, for example, 1-24 hours, or 1-7 days. The compound may be administered according to a schedule or the compound may be administered without a predetermined schedule. An active compound may be administered, for example, 1, 2, 3, 4, 5, 6, 7, 8, 9, 10, 11, or 12 times per day, every 2nd, 3rd, 4th, 5th, or 6th day, or 1, 2, 3, 4, 5, 6, or 7 times per week. In the case of parenteral administration, such as intravenous or subcutaneous administration, each compound or combination may be administered as bolus administration one or several times per day (1-12 times per day), or as slow bolus with each individual bolus administration taking 1 to 120 min, or as continuous infusion, without or with loading bolus administration. It is to be understood that, for any particular subject, specific dosage regimes should be adjusted over time according to the individual need and the professional judgment of the person administering or supervising the administration of the compositions.

An effective amount of each of the compounds of the combination may be, for example, a total daily dosage of, e.g., between 0.05 mg and 6000 mg of any of the compounds described herein. For some compounds, especially the antioxidant compound family, the total daily dose may exceed 3000 mg, with a top dose of up to 10000 mg. Alternatively, the dosage amount can be calculated using the body weight of the patient. Such dose ranges may include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the individual compound is administered.

The time periods during which the therapeutic combination may be administered may be as described herein.

According to a preferred embodiment, the treatment is to be administered within a defined time frame after the occurrence of the traumatic brain injury. The initiation treatment with effective doses is initiated within less than 7 days after the brain insult, preferably within less than 48 or 24 h after the brain insult, and most preferably within less than 8 h after the brain insult. The initial treatment is continued for a period of 3 day to 3 months after the insult, preferably for 5-30 days. The initiation treatment may be followed by a continuation treatment with the same combination immediately thereafter, either by a different administration route or using the same administration route. Such prolonged treatment can be expected to continue after the initiation treatment for 3 to 6 months, or, if epileptogenesis is only ameliorated, may be administered chronically, to treat the remaining symptoms, as medically indicated, or until occurrence of uncontrolled seizures, which require a change in treatment regimen.

Formulations

A compound identified as capable of treating any of the conditions described herein if administered in the invented combination of compounds, using any of the methods described herein, may be administered to patients or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to patients suffering from a bacterial infection. Administration may begin before the patient is symptomatic.

Exemplary routes of administration of the compounds described herein, or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The compounds desirably are administered with a pharmaceutically acceptable carrier. Pharmaceutical formulations of the compounds described herein formulated for treatment of the disorders described herein are also part of the present invention.

Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution- or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

Formulations for Buccal Administration

Dosages for buccal or sublingual administration typically are 0.1 to 500 mg per single dose as required. In practice, the physician determines the actual dosing regimen which is most suitable for an individual patient, and the dosage varies with the age, weight, and response of the particular patient. The above dosages are exemplary of the average case, but individual instances exist wherein higher or lower dosages are merited, and such are within the scope of this invention.

For buccal administration, the compositions may take the form of tablets, lozenges, etc. formulated in a conventional manner. Liquid drug formulations suitable for use with nebulizers and liquid spray devices and electrohydrodynamic (EHD) aerosol devices will typically include a compound with a pharmaceutically acceptable carrier. Preferably, the pharmaceutically acceptable carrier is a liquid, e.g., alcohol, water, polyethylene glycol, or a perfluorocarbon. Optionally, another material may be added to alter the aerosol properties of the solution or suspension of compounds. Desirably, this material is liquid, e.g., an alcohol, glycol, polyglycol, or a fatty acid. Other methods of formulating liquid drug solutions or suspension suitable for use in aerosol devices are known to those of skill in the art (see, e.g., Biesalski, U.S. Pat. No. 5,112,598 and Biesalski, U.S. Pat. No. 5,556,611, each of which is herein incorporated by reference).

Formulations for Nasal or Inhalation Administration

The compounds may also be formulated for nasal administration. Compositions for nasal administration also may conveniently be formulated as aerosols, drops, gels, and powders. The formulations may be provided in a single or multidose form. In the case of a dropper or pipette, dosing may be achieved by the patient administering an appropriate, predetermined volume of the solution or suspension. In the case of a spray, this may be achieved, for example, by means of a metering atomizing spray pump.

The compounds may further be formulated for aerosol administration, particularly to the respiratory tract by inhalation and including intranasal administration. The compound will generally have a small particle size for example on the order of five (5) microns or less. Such a particle size may be obtained by means known in the art, for example by micronization. The active ingredient is provided in a pressurized pack with a suitable propellant, e.g., a chlorofluorocarbon (CFC), for example, dichlorodifluoromethane, trichlorofluoromethane, or dichlorotetrafluoroethane, or carbon dioxide, or other suitable gas. The aerosol may conveniently also contain a surfactant, e.g., lecithin. The dose of drug may be controlled by a metered valve. Alternatively, the active ingredients may be provided in a form of a dry powder, e.g., a powder mix of the compound in a suitable powder base, e.g., lactose, starch, and starch derivatives, e.g., hydroxypropylmethyl cellulose, and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, e.g., a single dose nasal inhaler or an aerosol dispenser fitted with a metering valve which is intended for disposal after use. Where the dosage form comprises an aerosol dispenser, it will contain a propellant, which can be a compressed gas, e.g., compressed air or an organic propellant, e.g., fluorochlorohydrocarbon. The aerosol dosage forms can also take the form of a pump-atomizer.

Formulations for Parenteral Administration

The compounds described herein for use in the methods described herein can be administered in a pharmaceutically acceptable parenteral (e.g., intravenous or intramuscular or subcutaneous) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:

(1) “Drug Injection:” a liquid preparation that is a drug substance (e.g., a compound or combination of compounds described herein), or a solution thereof;

(2) “Drug for Injection:” the drug substance (e.g., a compound or combination of compounds described herein) as a dry solid that will be combined with the appropriate sterile vehicle for parenteral administration as a drug injection;

(3) “Drug Injectable Emulsion:” a liquid preparation of the drug substance (e.g., a compound or combination of compounds described herein) that is dissolved or dispersed in a suitable emulsion medium;

(4) “Drug Injectable Suspension:” a liquid preparation of the drug substance (e.g., a compound or combination of compounds described herein) suspended in a suitable liquid medium; and

(5) “Drug for Injectable Suspension:” the drug substance (e.g., a compound or combination of compounds described herein) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21st Ed., Gennaro, Ed., Lippincott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include, for example, aqueous solutions, powders for reconstitution, cosolvent solutions, oil/water emulsions, suspensions, oil-based solutions, liposomes, microspheres, and polymeric gels.

Preferred Pharmaceutical Compositions

In some embodiments, the invention provides liquid pharmaceutical compositions of topiramate or a pharmaceutically acceptable salt thereof. As is demonstrated herein, it was surprisingly found that liquid pharmaceutical compositions of topiramate could be prepared by including meglumine as a co-solvent in the composition. Such a liquid pharmaceutical composition disclosed herein includes topiramate (or a pharmaceutically acceptable salt of topiramate), meglumine, and a pharmaceutically acceptable excipient. Preferably, the liquid pharmaceutical composition is aqueous.

Topiramate

Liquid pharmaceutical compositions disclosed herein may include, e.g., 1 mg/mL to 100 mg/mL (e.g., 1 mg/mL to 100 mg/mL, 4 mg/mL to 100 mg/mL, 10 mg/mL to 100 mg/mL, 13 mg/mL to 100 mg/mL, 20 mg/mL to 100 mg/mL, 30 mg/mL to 100 mg/mL, 37 mg/mL to 100 mg/mL, 40 mg/mL to 100 mg/mL, 50 mg/mL to 100 mg/mL, 60 mg/mL to 100 mg/mL, 65 mg/mL to 100 mg/mL, 70 mg/mL to 100 mg/mL, 80 mg/mL to 100 mg/mL, 90 mg/mL to 100 mg/mL, 1 mg/mL to 90 mg/mL, 4 mg/mL to 90 mg/mL, 10 mg/mL to 90 mg/mL, 13 mg/mL to 90 mg/mL, 20 mg/mL to 90 mg/mL, 30 mg/mL to 90 mg/mL, 37 mg/mL to 90 mg/mL, 40 mg/mL to 90 mg/mL, 50 mg/mL to 90 mg/mL, 60 mg/mL to 90 mg/mL, 65 mg/mL to 90 mg/mL, 70 mg/mL to 90 mg/mL, 80 mg/mL to 90 mg/mL, 1 mg/mL to 80 mg/mL, 4 mg/mL to 80 mg/mL, 10 mg/mL to 80 mg/mL, 13 mg/mL to 80 mg/mL, 20 mg/mL to 80 mg/mL, 30 mg/mL to 80 mg/mL, 37 mg/mL to 80 mg/mL, 40 mg/mL to 80 mg/mL, 50 mg/mL to 80 mg/mL, 60 mg/mL to 80 mg/mL, 65 mg/mL to 80 mg/mL, 70 mg/mL to 80 mg/mL, 1 mg/mL to 70 mg/mL, 4 mg/mL to 70 mg/mL, 10 mg/mL to 70 mg/mL, 13 mg/mL to 70 mg/mL, 20 mg/mL to 70 mg/mL, 30 mg/mL to 70 mg/mL, 37 mg/mL to 70 mg/mL, 40 mg/mL to 70 mg/mL, 50 mg/mL to 70 mg/mL, 60 mg/mL to 70 mg/mL, 65 mg/mL to 70 mg/mL, 1 mg/mL to 65 mg/mL, 4 mg/mL to 65 mg/mL, 10 mg/mL to 65 mg/mL, 13 mg/mL to 65 mg/mL, 20 mg/mL to 65 mg/mL, 30 mg/mL to 65 mg/mL, 37 mg/mL to 65 mg/mL, 40 mg/mL to 65 mg/mL, 50 mg/mL to 65 mg/mL, 60 mg/mL to 65 mg/mL, 1 mg/mL to 60 mg/mL, 4 mg/mL to 60 mg/mL, 10 mg/mL to 60 mg/mL, 13 mg/mL to 60 mg/mL, 20 mg/mL to 60 mg/mL, 30 mg/mL to 60 mg/mL, 37 mg/mL to 60 mg/mL, 40 mg/mL to 60 mg/mL, 50 mg/mL to 60 mg/mL, 1 mg/mL to 50 mg/mL, 4 mg/mL to 50 mg/mL, 10 mg/mL to 50 mg/mL, 13 mg/mL to 50 mg/mL, 20 mg/mL to 50 mg/mL, 30 mg/mL to 50 mg/mL, 37 mg/mL to 50 mg/mL, 40 mg/mL to 50 mg/mL, 1 mg/mL to 40 mg/mL, 4 mg/mL to 40 mg/mL, 10 mg/mL to 40 mg/mL, 13 mg/mL to 40 mg/mL, 20 mg/mL to 40 mg/mL, 30 mg/mL to 40 mg/mL, 37 mg/mL to 40 mg/mL, 1 mg/mL to 37 mg/mL, 4 mg/mL to 37 mg/mL, 10 mg/mL to 37 mg/mL, 13 mg/mL to 37 mg/mL, 20 mg/mL to 37 mg/mL, 30 mg/mL to 37 mg/mL, 1 mg/mL to 30 mg/mL, 4 mg/mL to 30 mg/mL, 10 mg/mL to 30 mg/mL, 13 mg/mL to 30 mg/mL, 20 mg/mL to 30 mg/mL, 1 mg/mL to 20 mg/mL, 4 mg/mL to 20 mg/mL, 10 mg/mL to 20 mg/mL, 13 mg/mL to 20 mg/mL, 1 mg/mL to 13 mg/mL, 4 mg/mL to 13 mg/mL, 10 mg/mL to 13 mg/mL, 1 mg/mL to 10 mg/mL, 4 mg/mL to 10 mg/mL, or 1 mg/mL to 4 mg/mL) of topiramate or a pharmaceutically acceptable salt thereof.

Preferably, a liquid pharmaceutical composition disclosed herein includes 10 mg/mL to 70 mg/mL (e.g., 13 mg/mL to 70 mg/mL, 20 mg/mL to 70 mg/mL, 30 mg/mL to 70 mg/mL, 37 mg/mL to 70 mg/mL, 40 mg/mL to 70 mg/mL, 50 mg/mL to 70 mg/mL, 60 mg/mL to 70 mg/mL, 65 mg/mL to 70 mg/mL, 10 mg/mL to 65 mg/mL, 13 mg/mL to 65 mg/mL, 20 mg/mL to 65 mg/mL, 30 mg/mL to 65 mg/mL, 37 mg/mL to 65 mg/mL, 40 mg/mL to 65 mg/mL, 50 mg/mL to 65 mg/mL, 60 mg/mL to 65 mg/mL, 10 mg/mL to 60 mg/mL, 13 mg/mL to 60 mg/mL, 20 mg/mL to 60 mg/mL, 30 mg/mL to 60 mg/mL, 37 mg/mL to 60 mg/mL, 40 mg/mL to 60 mg/mL, 50 mg/mL to 60 mg/mL, 10 mg/mL to 50 mg/mL, 13 mg/mL to 50 mg/mL, 20 mg/mL to 50 mg/mL, 30 mg/mL to 50 mg/mL, 37 mg/mL to 50 mg/mL, 40 mg/mL to 50 mg/mL, 10 mg/mL to 40 mg/mL, 13 mg/mL to 40 mg/mL, 20 mg/mL to 40 mg/mL, 30 mg/mL to 40 mg/mL, 37 mg/mL to 40 mg/mL, 10 mg/mL to 37 mg/mL, 13 mg/mL to 37 mg/mL, 20 mg/mL to 37 mg/mL, 30 mg/mL to 37 mg/mL, 10 mg/mL to 30 mg/mL, 13 mg/mL to 30 mg/mL, 20 mg/mL to 30 mg/mL, 10 mg/mL to 20 mg/mL, 13 mg/mL to 20 mg/mL, or 10 mg/mL to 13 mg/mL) of topiramate or a pharmaceutically acceptable salt thereof.

More preferably, a liquid pharmaceutical composition disclosed herein includes 20 mg/mL to 65 mg/mL (e.g., 30 mg/mL to 65 mg/mL, 37 mg/mL to 65 mg/mL, 40 mg/mL to 65 mg/mL, 50 mg/mL to 65 mg/mL, 60 mg/mL to 65 mg/mL, 20 mg/mL to 60 mg/mL, 30 mg/mL to 60 mg/mL, 37 mg/mL to 60 mg/mL, 40 mg/mL to 60 mg/mL, 50 mg/mL to 60 mg/mL, 20 mg/mL to 50 mg/mL, 30 mg/mL to 50 mg/mL, 37 mg/mL to 50 mg/mL, 40 mg/mL to 50 mg/mL, 20 mg/mL to 40 mg/mL, 30 mg/mL to 40 mg/mL, 37 mg/mL to 40 mg/mL, 20 mg/mL to 37 mg/mL, 30 mg/mL to 37 mg/mL, or 20 mg/mL to 30 mg/mL) of topiramate or a pharmaceutically acceptable salt thereof.

In some preferred embodiments (e.g., in instances where a lower concentration of meglumine is preferable, such as in liquid pharmaceutical compositions containing a combination of therapeutic agents described herein), a liquid pharmaceutical composition disclosed herein includes 4 mg/mL to 30 mg/mL (e.g., 10 mg/mL to 30 mg/mL, 13 mg/mL to 30 mg/mL, 20 mg/mL to 30 mg/mL, 4 mg/mL to 20 mg/mL, 10 mg/mL to 20 mg/mL, 13 mg/mL to 20 mg/mL, 4 mg/mL to 13 mg/mL, 10 mg/mL to 13 mg/mL, or 4 mg/mL to 10 mg/mL) of topiramate or a pharmaceutically acceptable salt thereof. In certain more preferred embodiments (e.g., in instances where a lower concentration of meglumine (e.g., 1 mg/mL to 50 mg/mL (preferably, 5 mg/mL to 50 mg/mL)) is preferable, such as in liquid pharmaceutical compositions containing a combination of therapeutic agents described herein), a liquid pharmaceutical composition disclosed herein includes 4 mg/mL to 20 mg/mL (e.g., 4 mg/mL to 9 mg/mL, 9 mg/mL to 20 mg/mL, 4 mg/mL to 7 mg/mL, 7 mg/mL to 15 mg/mL, or 8 mg/mL to 12 mg/mL) of topiramate or a pharmaceutically acceptable salt thereof. In even more preferred embodiments (e.g., in instances where a lower concentration of meglumine is preferable, such as in liquid pharmaceutical compositions containing a combination of therapeutic agents described herein), a liquid pharmaceutical composition disclosed herein includes 9 mg/mL to 11 mg/mL (e.g., 10 mg/mL) of topiramate or a pharmaceutically acceptable salt thereof. In alternative, preferred embodiments (e.g., for liquid pharmaceutical compositions configured for administration at a higher dosage volume (e.g., 40 mL to 50 mL), a liquid pharmaceutical composition disclosed herein includes 4 mg/mL to 6 mg/mL (e.g., 5 mg/mL) of topiramate or a pharmaceutically acceptable salt thereof.

Meglumine

Liquid pharmaceutical composition disclosed herein may include, e.g., 1 mg/mL to 550 mg/mL (e.g., 1 mg/mL to 500 mg/mL, 1 mg/mL to 400 mg/mL, 1 mg/ml to 300 mg/mL, 1 mg/ml to 250 mg/mL, 1 mg/mL to 200 mg/mL, 1 mg/mL to 150 mg/mL, 1 mg/mL to 100 mg/mL, 1 mg/mL to 75 mg/ml, 1 mg/mL to 60 mg/mL, 1 mg/mL to 50 mg/mL, 1 mg/mL to 30 mg/mL, 1 mg/mL to 25 mg/mL, 1 mg/mL to 10 mg/mL, 5 mg/mL to 550 mg/mL, 5 mg/mL to 500 mg/mL, 5 mg/mL to 400 mg/mL, 5 mg/mL to 300 mg/mL, 5 mg/mL to 250 mg/mL, 5 mg/mL to 200 mg/mL, 5 mg/mL to 150 mg/mL, 5 mg/mL to 100 mg/mL, 5 mg/mL to 75 mg/mL, 5 mg/mL to 60 mg/mL, 5 mg/mL to 50 mg/mL, 5 mg/mL to 30 mg/mL, 5 mg/mL to 25 mg/mL, 5 mg/mL to 10 mg/mL, 10 mg/mL to 550 mg/mL, 10 mg/mL to 500 mg/mL, 10 mg/mL to 400 mg/mL, 10 mg/mL to 300 mg/mL, 10 mg/mL to 250 mg/mL, 10 mg/mL to 200 mg/mL, 10 mg/mL to 150 mg/mL, 10 mg/mL to 100 mg/mL, 10 mg/mL to 75 mg/mL, 10 mg/mL to 60 mg/mL, 10 mg/mL to 50 mg/mL, 10 mg/mL to 30 mg/mL, 10 mg/mL to 25 mg/mL, 25 mg/mL to 550 mg/mL, 25 mg/mL to 500 mg/mL, 25 mg/mL to 400 mg/mL, 25 mg/mL to 300 mg/mL, 25 mg/mL to 250 mg/mL, 25 mg/mL to 200 mg/mL, 25 mg/mL to 150 mg/mL, 25 mg/mL to 100 mg/mL, 25 mg/mL to 75 mg/mL, 25 mg/mL to 60 mg/mL, 25 mg/mL to 50 mg/mL, 25 mg/mL to 30 mg/mL, 50 mg/mL to 550 mg/mL, 50 mg/mL to 500 mg/mL, 50 mg/mL to 400 mg/mL, 50 mg/mL to 300 mg/mL, 50 mg/mL to 250 mg/mL, 50 mg/mL to 200 mg/mL, 50 mg/mL to 150 mg/mL, 50 mg/mL to 100 mg/mL, 50 mg/mL to 75 mg/mL, 50 mg/mL to 60 mg/mL, 60 mg/mL to 550 mg/mL, 60 mg/mL to 500 mg/mL, 60 mg/mL to 400 mg/mL, 60 mg/mL to 300 mg/mL, 60 mg/mL to 250 mg/mL, 60 mg/mL to 200 mg/mL, 60 mg/mL to 150 mg/mL, 60 mg/mL to 100 mg/mL, 60 mg/mL to 75 mg/mL, 75 mg/mL to 550 mg/mL, 75 mg/mL to 500 mg/mL, 75 mg/mL to 400 mg/mL, 75 mg/mL to 300 mg/mL, 75 mg/mL to 250 mg/mL, 75 mg/mL to 200 mg/mL, 75 mg/mL to 150 mg/mL, 75 mg/mL to 100 mg/mL, 100 mg/mL to 550 mg/mL, 100 mg/mL to 500 mg/mL, 100 mg/mL to 400 mg/mL, 100 mg/mL to 300 mg/mL, 100 mg/mL to 250 mg/mL, 100 mg/mL to 200 mg/mL, 100 mg/mL to 150 mg/mL, 150 mg/mL to 550 mg/mL, 150 mg/mL to 500 mg/mL, 150 mg/mL to 400 mg/mL, 150 mg/mL to 300 mg/mL, 150 mg/mL to 250 mg/mL, 150 mg/mL to 200 mg/mL, 200 mg/mL to 550 mg/mL, 200 mg/mL to 500 mg/mL, 200 mg/mL to 400 mg/mL, 200 mg/mL to 300 mg/mL, 200 mg/mL to 250 mg/mL, 250 mg/mL to 550 mg/mL, 250 mg/mL to 500 mg/mL, 250 mg/mL to 400 mg/mL, 250 mg/mL to 300 mg/mL, 300 mg/mL to 550 mg/mL, 300 mg/mL to 500 mg/mL, 300 mg/mL to 400 mg/mL, 400 mg/mL to 550 mg/mL, or 400 mg/mL to 500 mg/mL) of meglumine.

Liquid pharmaceutical compositions disclosed herein may include, e.g., 1 mg/mL to 100 mg/mL (e.g., 1 mg/mL to 90 mg/mL, 1 mg/mL to 80 mg/mL, 1 mg/mL to 70 mg/mL, 1 mg/mL to 60 mg/mL, 1 mg/mL to 50 mg/mL, 1 mg/mL to 40 mg/mL, 1 mg/mL to 30 mg/mL, 1 mg/mL to 25 mg/mL, 1 mg/mL to 20 mg/mL, 1 mg/mL to 15 mg/mL, 1 mg/mL to 10 mg/mL, 1 mg/mL to 5 mg/mL, 2 mg/mL to 100 mg/mL, 2 mg/mL to 90 mg/mL, 2 mg/mL to 80 mg/mL, 2 mg/mL to 70 mg/mL, 2 mg/mL to 60 mg/mL, 2 mg/mL to 50 mg/mL, 2 mg/mL to 40 mg/mL, 2 mg/mL to 30 mg/mL, 2 mg/mL to 25 mg/mL, 2 mg/mL to 20 mg/mL, 2 mg/mL to 15 mg/mL, 2 mg/mL to 10 mg/mL, 2 mg/mL to 5 mg/mL, 3 mg/mL to 100 mg/mL, 3 mg/mL to 90 mg/mL, 3 mg/mL to 80 mg/mL, 3 mg/mL to 70 mg/mL, 3 mg/mL to 60 mg/mL, 3 mg/mL to 50 mg/mL, 3 mg/mL to 40 mg/mL, 3 mg/mL to 30 mg/mL, 3 mg/mL to 25 mg/mL, 3 mg/mL to 20 mg/mL, 3 mg/mL to 15 mg/mL, 3 mg/mL to 10 mg/mL, 3 mg/mL to 5 mg/mL, 4 mg/mL to 100 mg/mL, 4 mg/mL to 90 mg/mL, 4 mg/mL to 80 mg/mL, 4 mg/mL to 70 mg/mL, 4 mg/mL to 60 mg/mL, 4 mg/mL to 50 mg/mL, 4 mg/mL to 40 mg/mL, 4 mg/mL to 30 mg/mL, 4 mg/mL to 25 mg/mL, 4 mg/mL to 20 mg/mL, 4 mg/mL to 15 mg/mL, 4 mg/mL to 10 mg/mL, 4 mg/mL to 5 mg/mL, 5 mg/mL to 100 mg/mL, 5 mg/mL to 90 mg/mL, 5 mg/mL to 80 mg/mL, 5 mg/mL to 70 mg/mL, 5 mg/mL to 60 mg/mL, 5 mg/mL to 50 mg/mL, 5 mg/mL to 40 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 100 mg/mL, 10 mg/mL to 90 mg/mL, 10 mg/mL to 80 mg/mL, 10 mg/mL to 70 mg/mL, 10 mg/mL to 60 mg/mL, 10 mg/mL to 50 mg/mL, 10 mg/mL to 40 mg/mL, 10 mg/mL to 30 mg/mL, 10 mg/mL to 25 mg/mL, 10 mg/mL to 20 mg/mL, 10 mg/mL to 15 mg/mL, 15 mg/mL to 100 mg/mL, 15 mg/mL to 90 mg/mL, 15 mg/mL to 80 mg/mL, 15 mg/mL to 70 mg/mL, 15 mg/mL to 60 mg/mL, 15 mg/mL to 50 mg/mL, 15 mg/mL to 40 mg/mL, 15 mg/mL to 30 mg/mL, 15 mg/mL to 25 mg/mL, 15 mg/mL to 20 mg/mL, 20 mg/mL to 100 mg/mL, 20 mg/mL to 90 mg/mL, 20 mg/mL to 80 mg/mL, 20 mg/mL to 70 mg/mL, 20 mg/mL to 60 mg/mL, 20 mg/mL to 50 mg/mL, 20 mg/mL to 40 mg/mL, 20 mg/mL to 30 mg/mL, 20 mg/mL to 25 mg/mL, 25 mg/mL to 100 mg/mL, 25 mg/mL to 90 mg/mL, 25 mg/mL to 80 mg/mL, 25 mg/mL to 70 mg/mL, 25 mg/mL to 60 mg/mL, 25 mg/mL to 50 mg/mL, 25 mg/mL to 40 mg/mL, 25 mg/mL to 30 mg/mL, 30 mg/mL to 100 mg/mL, 30 mg/mL to 90 mg/mL, 30 mg/mL to 80 mg/mL, 30 mg/mL to 70 mg/mL, 30 mg/mL to 60 mg/mL, 30 mg/mL to 50 mg/mL, 30 mg/mL to 40 mg/mL, 40 mg/mL to 100 mg/mL, 40 mg/mL to 90 mg/mL, 40 mg/mL to 80 mg/mL, 40 mg/mL to 70 mg/mL, 40 mg/mL to 60 mg/mL, 40 mg/mL to 50 mg/mL, 50 mg/mL to 100 mg/mL, 50 mg/mL to 90 mg/mL, 50 mg/mL to 80 mg/mL, 50 mg/mL to 70 mg/mL, 50 mg/mL to 60 mg/mL, 60 mg/mL to 100 mg/mL, 60 mg/mL to 90 mg/mL, 60 mg/mL to 80 mg/mL, 60 mg/mL to 70 mg/mL, 70 mg/mL to 100 mg/mL, 70 mg/mL to 90 mg/mL, 70 mg/mL to 80 mg/mL, 80 mg/mL to 100 mg/mL, 80 mg/mL to 90 mg/mL, or 90 mg/mL to 100 mg/mL) of meglumine.

Preferably, a liquid pharmaceutical composition disclosed herein includes 1 mg/mL to 50 mg/mL (e.g., 1 mg/mL to 40 mg/mL, 1 mg/mL to 30 mg/mL, 1 mg/mL to 25 mg/mL, 1 mg/mL to 20 mg/mL, 1 mg/mL to 15 mg/mL, 1 mg/mL to 10 mg/mL, 1 mg/mL to 5 mg/mL, 2 mg/mL to 50 mg/mL, 2 mg/mL to 40 mg/mL, 2 mg/mL to 30 mg/mL, 2 mg/mL to 25 mg/mL, 2 mg/mL to 20 mg/mL, 2 mg/mL to 15 mg/mL, 2 mg/mL to 10 mg/mL, 2 mg/mL to 5 mg/mL, 3 mg/mL to 50 mg/mL, 3 mg/mL to 40 mg/mL, 3 mg/mL to 30 mg/mL, 3 mg/mL to 25 mg/mL, 3 mg/mL to 20 mg/mL, 3 mg/mL to 15 mg/mL, 3 mg/mL to 10 mg/mL, 3 mg/mL to 5 mg/mL, 4 mg/mL to 50 mg/mL, 4 mg/mL to 40 mg/mL, 4 mg/mL to 30 mg/mL, 4 mg/mL to 25 mg/mL, 4 mg/mL to 20 mg/mL, 4 mg/mL to 15 mg/mL, 4 mg/mL to 10 mg/mL, 4 mg/mL to 5 mg/mL, 5 mg/mL to 50 mg/mL, 5 mg/mL to 40 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 50 mg/mL, 10 mg/mL to 40 mg/mL, 10 mg/mL to 30 mg/mL, 10 mg/mL to 25 mg/mL, 10 mg/mL to 20 mg/mL, 10 mg/mL to 15 mg/mL, 15 mg/mL to 50 mg/mL, 15 mg/mL to 40 mg/mL, 15 mg/mL to 30 mg/mL, 15 mg/mL to 25 mg/mL, 15 mg/mL to 20 mg/mL, 20 mg/mL to 50 mg/mL, 20 mg/mL to 40 mg/mL, 20 mg/mL to 30 mg/mL, 20 mg/mL to 25 mg/mL, 25 mg/mL to 50 mg/mL, 25 mg/mL to 40 mg/mL, 25 mg/mL to 30 mg/mL, 30 mg/mL to 50 mg/mL, 30 mg/mL to 40 mg/mL, or 40 mg/mL to 50 mg/mL) of meglumine. More preferably, a liquid pharmaceutical composition disclosed herein includes 5 mg/mL to 50 mg/mL of meglumine.

In certain preferred embodiments (e.g., for liquid pharmaceutical compositions including two or more therapeutic agents described herein), a liquid pharmaceutical composition disclosed herein includes 1 mg/mL to 30 mg/mL (e.g., 1 mg/mL to 25 mg/mL, 1 mg/mL to 20 mg/mL, 1 mg/mL to 15 mg/mL, 1 mg/mL to 10 mg/mL, 1 mg/mL to 5 mg/mL, 2 mg/mL to 30 mg/mL, 2 mg/mL to 25 mg/mL, 2 mg/mL to 20 mg/mL, 2 mg/mL to 15 mg/mL, 2 mg/mL to 10 mg/mL, 2 mg/mL to 5 mg/mL, 3 mg/mL to 30 mg/mL, 3 mg/mL to 25 mg/mL, 3 mg/mL to 20 mg/mL, 3 mg/mL to 15 mg/mL, 3 mg/mL to 10 mg/mL, 3 mg/mL to 5 mg/mL, 4 mg/mL to 30 mg/mL, 4 mg/mL to 25 mg/mL, 4 mg/mL to 20 mg/mL, 4 mg/mL to 15 mg/mL, 4 mg/mL to 10 mg/mL, 4 mg/mL to 5 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 30 mg/mL, 10 mg/mL to 25 mg/mL, 10 mg/mL to 20 mg/mL, 10 mg/mL to 15 mg/mL, 15 mg/mL to 30 mg/mL, 15 mg/mL to 25 mg/mL, 15 mg/mL to 20 mg/mL, 20 mg/mL to 30 mg/mL, 20 mg/mL to 25 mg/mL, or 25 mg/mL to 30 mg/mL) of meglumine.

In further preferred embodiments (e.g., for liquid pharmaceutical compositions described herein including topiramate as the only therapeutic agent), a liquid pharmaceutical composition disclosed herein includes 10 mg/mL to 50 mg/mL (e.g., 10 mg/mL to 40 mg/mL, 10 mg/mL to 30 mg/mL, 10 mg/mL to 25 mg/mL, 10 mg/mL to 20 mg/mL, 10 mg/mL to 15 mg/mL, 15 mg/mL to 50 mg/mL, 15 mg/mL to 40 mg/mL, 15 mg/mL to 30 mg/mL, 15 mg/mL to 25 mg/mL, 15 mg/mL to 20 mg/mL, 20 mg/mL to 50 mg/mL, 20 mg/mL to 40 mg/mL, 20 mg/mL to 30 mg/mL, 20 mg/mL to 25 mg/mL, 25 mg/mL to 50 mg/mL, 25 mg/mL to 40 mg/mL, 25 mg/mL to 30 mg/mL, 30 mg/mL to 50 mg/mL, 30 mg/mL to 40 mg/mL, or 40 mg/mL to 50 mg/mL) of meglumine.

Further Therapeutic Agents

Levetiracetam

Liquid pharmaceutical compositions disclosed herein may include one or more further therapeutic agents in addition to topiramate or a pharmaceutically acceptable salt thereof.

Preferably, a liquid pharmaceutical composition disclosed herein further includes levetiracetam. For example, a liquid pharmaceutical composition disclosed herein may include 5 mg/mL to 500 mg/mL (e.g., 5 mg/mL to 400 mg/mL, 5 mg/mL to 300 mg/mL, 5 mg/mL to 250 mg/mL, 5 mg/mL to 200 mg/mL, 5 mg/mL to 150 mg/mL, 5 mg/mL to 100 mg/mL, 5 mg/mL to 75 mg/mL, 5 mg/mL to 60 mg/mL, 5 mg/mL to 50 mg/mL, 5 mg/mL to 30 mg/mL, 5 mg/mL to 25 mg/mL, 5 mg/mL to 10 mg/mL, 10 mg/mL to 500 mg/mL, 10 mg/mL to 400 mg/mL, 10 mg/mL to 300 mg/mL, 10 mg/mL to 250 mg/mL, 10 mg/mL to 200 mg/mL, 10 mg/mL to 150 mg/mL, 10 mg/mL to 100 mg/mL, 10 mg/mL to 75 mg/mL, 10 mg/mL to 60 mg/mL, 10 mg/mL to 50 mg/mL, 10 mg/mL to 30 mg/mL, 10 mg/mL to 25 mg/mL, 25 mg/mL to 500 mg/mL, 25 mg/mL to 400 mg/mL, 25 mg/mL to 300 mg/mL, 25 mg/mL to 250 mg/mL, 25 mg/mL to 200 mg/mL, 25 mg/mL to 150 mg/mL, 25 mg/mL to 100 mg/mL, 25 mg/mL to 75 mg/mL, 25 mg/mL to 60 mg/mL, 25 mg/mL to 50 mg/mL, 25 mg/mL to 30 mg/mL, 50 mg/mL to 500 mg/mL, 50 mg/mL to 400 mg/mL, 50 mg/mL to 300 mg/mL, 50 mg/mL to 250 mg/mL, 50 mg/mL to 200 mg/mL, 50 mg/mL to 150 mg/mL, 50 mg/mL to 100 mg/mL, 50 mg/mL to 75 mg/mL, 50 mg/mL to 60 mg/mL, 60 mg/mL to 500 mg/mL, 60 mg/mL to 400 mg/mL, 60 mg/mL to 300 mg/mL, 60 mg/mL to 250 mg/mL, 60 mg/mL to 200 mg/mL, 60 mg/mL to 150 mg/mL, 60 mg/mL to 100 mg/mL, 60 mg/mL to 75 mg/mL, 75 mg/mL to 500 mg/mL, 75 mg/mL to 400 mg/mL, 75 mg/mL to 300 mg/mL, 75 mg/mL to 250 mg/mL, 75 mg/mL to 200 mg/mL, 75 mg/mL to 150 mg/mL, 75 mg/mL to 100 mg/mL, 100 mg/mL to 500 mg/mL, 100 mg/mL to 400 mg/mL, 100 mg/mL to 300 mg/mL, 100 mg/mL to 250 mg/mL, 100 mg/mL to 200 mg/mL, 100 mg/mL to 150 mg/mL, 150 mg/mL to 500 mg/mL, 150 mg/mL to 400 mg/mL, 150 mg/mL to 300 mg/mL, 150 mg/mL to 250 mg/mL, 150 mg/mL to 200 mg/mL, 200 mg/mL to 500 mg/mL, 200 mg/mL to 400 mg/mL, 200 mg/mL to 300 mg/mL, 200 mg/mL to 250 mg/mL, 250 mg/mL to 500 mg/mL, 250 mg/mL to 400 mg/mL, 250 mg/mL to 300 mg/mL, 300 mg/mL to 500 mg/mL, 300 mg/mL to 400 mg/mL, or 400 mg/mL to 500 mg/mL) of levetiracetam.

In certain preferred embodiments, a liquid pharmaceutical composition disclosed herein includes 5 mg/mL to 150 mg/mL (e.g., 5 mg/mL to 100 mg/mL, 5 mg/mL to 75 mg/mL, 5 mg/mL to 60 mg/mL, 5 mg/mL to 50 mg/mL, 5 mg/mL to 30 mg/mL, 5 mg/mL to 25 mg/mL, 5 mg/mL to 10 mg/mL, 10 mg/mL to 150 mg/mL, 10 mg/mL to 100 mg/mL, 10 mg/mL to 75 mg/mL, 10 mg/mL to 60 mg/mL, 10 mg/mL to 50 mg/mL, 10 mg/mL to 30 mg/mL, 10 mg/mL to 25 mg/mL, 25 mg/mL to 150 mg/mL, 25 mg/mL to 100 mg/mL, 25 mg/mL to 75 mg/mL, 25 mg/mL to 60 mg/mL, 25 mg/mL to 50 mg/mL, 25 mg/mL to 30 mg/mL, 50 mg/mL to 150 mg/mL, 50 mg/mL to 100 mg/mL, 50 mg/mL to 75 mg/mL, 50 mg/mL to 60 mg/mL, 60 mg/mL to 150 mg/mL, 60 mg/mL to 100 mg/mL, 60 mg/mL to 75 mg/mL, 75 mg/mL to 150 mg/mL, 75 mg/mL to 100 mg/mL, or 100 mg/mL to 150 mg/mL) of levetiracetam. In some more preferred embodiments, a liquid pharmaceutical composition disclosed herein includes 20 mg/mL to 150 mg/mL (e.g., 20 mg/mL to 120 mg/mL, 20 mg/mL to 100 mg/mL, 20 mg/mL to 60 mg/mL, 20 mg/mL to 50 mg/mL, 50 mg/mL to 150 mg/mL, 50 mg/mL to 120 mg/mL, 50 mg/mL to 100 mg/mL, 50 mg/mL to 60 mg/mL, 100 mg/mL to 150 mg/mL, 100 mg/mL to 120 mg/mL, or 120 mg/mL to 150 mg/mL) of levetiracetam. In yet more preferred embodiments, a liquid pharmaceutical composition disclosed herein includes 60 mg/mL to 120 mg/mL of levetiracetam. In alternative yet more preferred embodiments (e.g., for liquid pharmaceutical compositions configured for administration at a higher dosage volume (e.g., 40 mL to 50 mL)), a liquid pharmaceutical composition disclosed herein includes 30 mg/mL to 60 mg/mL of levetiracetam.

Brivaracetam

In the alternative preferred embodiments, a liquid pharmaceutical composition disclosed herein further includes brivaracetam. For example, a liquid pharmaceutical composition disclosed herein may include 0.5 mg/mL to 50 mg/mL (e.g., 0.5 mg/mL to 40 mg/mL, 0.5 mg/mL to 30 mg/mL, 0.5 mg/mL to 25 mg/mL, 0.5 mg/mL to 20 mg/mL, 0.5 mg/mL to 15 mg/mL, 0.5 mg/mL to 10 mg/mL, 0.5 mg/mL to 7.5 mg/mL, 0.5 mg/mL to 5 mg/mL, 0.5 mg/mL to 2.5 mg/mL, 2.5 mg/mL to 50 mg/mL, 2.5 mg/mL to 40 mg/mL, 2.5 mg/mL to 30 mg/mL, 2.5 mg/mL to 25 mg/mL, 2.5 mg/mL to 20 mg/mL, 2.5 mg/mL to 15 mg/mL, 2.5 mg/mL to 10 mg/mL, 2.5 mg/mL to 7.5 mg/mL, 2.5 mg/mL to 5 mg/mL, 5 mg/mL to 50 mg/mL, 5 mg/mL to 40 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 5 mg/mL to 7.5 mg/mL, 7.5 mg/mL to 50 mg/mL, 7.5 mg/mL to 40 mg/mL, 7.5 mg/mL to 30 mg/mL, 7.5 mg/mL to 25 mg/mL, 7.5 mg/mL to 20 mg/mL, 7.5 mg/mL to 15 mg/mL, 7.5 mg/mL to 10 mg/mL, 10 mg/mL to 50 mg/mL, 10 mg/mL to 40 mg/mL, 10 mg/mL to 30 mg/mL, 10 mg/mL to 25 mg/mL, 10 mg/mL to 20 mg/mL, 10 mg/mL to 15 mg/mL, 15 mg/mL to 50 mg/mL, 15 mg/mL to 40 mg/mL, 15 mg/mL to 30 mg/mL, 15 mg/mL to 25 mg/mL, 15 mg/mL to 20 mg/mL, 20 mg/mL to 50 mg/mL, 20 mg/mL to 40 mg/mL, 20 mg/mL to 30 mg/mL, 20 mg/mL to 25 mg/mL, 25 mg/mL to 50 mg/mL, 25 mg/mL to 40 mg/mL, 25 mg/mL to 30 mg/mL, 30 mg/mL to 50 mg/mL, 30 mg/mL to 40 mg/mL, or 40 mg/mL to 50 mg/mL) of brivaracetam. In certain preferred embodiments, a liquid pharmaceutical composition disclosed herein includes 0.5 mg/mL to 10 mg/mL (e.g., 0.5 mg/mL to 7.5 mg/mL, 0.5 mg/mL to 5 mg/mL, 0.5 mg/mL to 2.5 mg/mL, 2.5 mg/mL to 10 mg/mL, 2.5 mg/mL to 7.5 mg/mL, 2.5 mg/mL to 5 mg/mL, 5 mg/mL to 10 mg/mL, 5 mg/mL to 7.5 mg/mL, or 7.5 mg/mL to 10 mg/mL) of brivaracetam. In yet more preferred embodiments, a liquid pharmaceutical composition disclosed herein includes 2 mg/mL to 8 mg/mL of brivaracetam. In still more preferred embodiments, a liquid pharmaceutical composition disclosed herein includes 4 mg/mL to 8 mg/mL of brivaracetam. In alternative yet more preferred embodiments (e.g., for liquid pharmaceutical compositions configured for administration at a higher dosage volume (e.g., 40 mL to 50 mL)), a liquid pharmaceutical composition disclosed herein includes 2 mg/mL to 4 mg/mL of brivaracetam.

Seletracetam

In the alternative preferred embodiments, a liquid pharmaceutical composition disclosed herein further includes seletracetam. For example, a liquid pharmaceutical composition disclosed herein may include 0.2 mg/mL to 20 mg/mL (e.g., 0.2 mg/mL to 15 mg/mL, 0.2 mg/mL to 12 mg/mL, 0.2 mg/mL to 10 mg/mL, 0.2 mg/mL to 7.5 mg/mL, 0.2 mg/mL to 5 mg/mL, 0.2 mg/mL to 3.3 mg/mL, 0.2 mg/mL to 2.5 mg/mL, 0.2 mg/mL to 1 mg/mL, 0.1 mg/mL to 0.5 mg/mL, 1 mg/mL to 20 mg/mL, 1 mg/mL to 15 mg/mL, 1 mg/mL to 12 mg/mL, 1 mg/mL to 10 mg/mL, 1 mg/mL to 7.5 mg/mL, 1 mg/mL to 5 mg/mL, 1 mg/mL to 3.3 mg/mL, 1 mg/mL to 2 mg/mL, 1 mg/mL to 1.5 mg/mL, 2 mg/mL to 20 mg/mL, 2 mg/mL to 15 mg/mL, 2 mg/mL to 12 mg/mL, 2 mg/mL to 10 mg/mL, 2 mg/mL to 7.5 mg/mL, 2 mg/mL to 5 mg/mL, 2 mg/mL to 3.3 mg/mL, 3.3 mg/mL to 20 mg/mL, 3.3 mg/mL to 15 mg/mL, 3.3 mg/mL to 10 mg/mL, 3.3 mg/mL to 7.5 mg/mL, 3.3 mg/mL to 5 mg/mL, 5 mg/mL to 20 mg/mL, 5 mg/mL to 15 mg/mL, 5 mg/mL to 12 mg/mL, 5 mg/mL to 10 mg/mL, 5 mg/mL to 7.5 mg/mL, 7.5 mg/mL to 20 mg/mL, 7.5 mg/mL to 15 mg/mL, 7.5 mg/mL to 12 mg/mL, 7.5 mg/mL to 10 mg/mL, 10 mg/mL to 20 mg/mL, 10 mg/mL to 15 mg/mL, 15 mg/mL to 20 mg/mL) of seletracetam. In certain preferred embodiments, a liquid pharmaceutical composition disclosed herein includes 0.2 mg/mL to 5 mg/mL (e.g., 0.2 mg/mL to 4 mg/mL, 0.2 mg/mL to 2.5 mg/mL, 0.2 mg/mL to 1 mg/mL, 0.2 mg/mL to 0.5 mg/mL, 1 mg/mL to 3.3 mg/mL, 1 mg/mL to 3.3 mg/mL, or 1 mg/mL to 2 mg/mL) of seletracetam. In yet more preferred embodiments, a liquid pharmaceutical composition disclosed herein includes 0.8 mg/mL to 3 mg/mL of seletracetam. In still more preferred embodiments, a liquid pharmaceutical composition disclosed herein includes 1.6 mg/mL to 3 mg/mL of seletracetam. In alternative yet more preferred embodiments (e.g., for liquid pharmaceutical compositions configured for administration at a higher dosage volume (e.g., 40 mL to 50 mL)), a liquid pharmaceutical composition disclosed herein includes 0.8 mg/mL to 1.6 mg/mL of seletracetam.

Padsevonil

In the alternative preferred embodiments, a liquid pharmaceutical composition disclosed herein further includes pdsevonil. For example, a liquid pharmaceutical composition disclosed herein may include 1 mg/mL to 100 mg/mL 1 mg/mL to 100 mg/mL (e.g., 1 mg/mL to 90 mg/mL, 1 mg/mL to 80 mg/mL, 1 mg/mL to 70 mg/mL, 1 mg/mL to 60 mg/mL, 1 mg/mL to 50 mg/mL, 1 mg/mL to 40 mg/mL, 1 mg/mL to 30 mg/mL, 1 mg/mL to 25 mg/mL, 1 mg/mL to 20 mg/mL, 1 mg/mL to 15 mg/mL, 1 mg/mL to 10 mg/mL, 1 mg/mL to 5 mg/mL, 2 mg/mL to 100 mg/mL, 2 mg/mL to 90 mg/mL, 2 mg/mL to 80 mg/mL, 2 mg/mL to 70 mg/mL, 2 mg/mL to 60 mg/mL, 2 mg/mL to 50 mg/mL, 2 mg/mL to 40 mg/mL, 2 mg/mL to 30 mg/mL, 2 mg/mL to 25 mg/mL, 2 mg/mL to 20 mg/mL, 2 mg/mL to 15 mg/mL, 2 mg/mL to 10 mg/mL, 2 mg/mL to 5 mg/mL, 3 mg/mL to 100 mg/mL, 3 mg/mL to 90 mg/mL, 3 mg/mL to 80 mg/mL, 3 mg/mL to 70 mg/mL, 3 mg/mL to 60 mg/mL, 3 mg/mL to 50 mg/mL, 3 mg/mL to 40 mg/mL, 3 mg/mL to 30 mg/mL, 3 mg/mL to 25 mg/mL, 3 mg/mL to 20 mg/mL, 3 mg/mL to 15 mg/mL, 3 mg/mL to 10 mg/mL, 3 mg/mL to 5 mg/mL, 4 mg/mL to 100 mg/mL, 4 mg/mL to 90 mg/mL, 4 mg/mL to 80 mg/mL, 4 mg/mL to 70 mg/mL, 4 mg/mL to 60 mg/mL, 4 mg/mL to 50 mg/mL, 4 mg/mL to 40 mg/mL, 4 mg/mL to 30 mg/mL, 4 mg/mL to 25 mg/mL, 4 mg/mL to 20 mg/mL, 4 mg/mL to 15 mg/mL, 4 mg/mL to 10 mg/mL, 4 mg/mL to 5 mg/mL, 5 mg/mL to 100 mg/mL, 5 mg/mL to 90 mg/mL, 5 mg/mL to 80 mg/mL, 5 mg/mL to 70 mg/mL, 5 mg/mL to 60 mg/mL, 5 mg/mL to 50 mg/mL, 5 mg/mL to 40 mg/mL, 5 mg/mL to 30 mg/mL, 5 mg/mL to 25 mg/mL, 5 mg/mL to 20 mg/mL, 5 mg/mL to 15 mg/mL, 5 mg/mL to 10 mg/mL, 10 mg/mL to 100 mg/mL, 10 mg/mL to 90 mg/mL, 10 mg/mL to 80 mg/mL, 10 mg/mL to 70 mg/mL, 10 mg/mL to 60 mg/mL, 10 mg/mL to 50 mg/mL, 10 mg/mL to 40 mg/mL, 10 mg/mL to 30 mg/mL, 10 mg/mL to 25 mg/mL, 10 mg/mL to 20 mg/mL, 10 mg/mL to 15 mg/mL, 15 mg/mL to 100 mg/mL, 15 mg/mL to 90 mg/mL, 15 mg/mL to 80 mg/mL, 15 mg/mL to 70 mg/mL, 15 mg/mL to 60 mg/mL, 15 mg/mL to 50 mg/mL, 15 mg/mL to 40 mg/mL, 15 mg/mL to 30 mg/mL, 15 mg/mL to 25 mg/mL, 15 mg/mL to 20 mg/mL, 20 mg/mL to 100 mg/mL, 20 mg/mL to 90 mg/mL, 20 mg/mL to 80 mg/mL, 20 mg/mL to 70 mg/mL, 20 mg/mL to 60 mg/mL, 20 mg/mL to 50 mg/mL, 20 mg/mL to 40 mg/mL, 20 mg/mL to 30 mg/mL, 20 mg/mL to 25 mg/mL, 25 mg/mL to 100 mg/mL, 25 mg/mL to 90 mg/mL, 25 mg/mL to 80 mg/mL, 25 mg/mL to 70 mg/mL, 25 mg/mL to 60 mg/mL, 25 mg/mL to 50 mg/mL, 25 mg/mL to 40 mg/mL, 25 mg/mL to 30 mg/mL, 30 mg/mL to 100 mg/mL, 30 mg/mL to 90 mg/mL, 30 mg/mL to 80 mg/mL, 30 mg/mL to 70 mg/mL, 30 mg/mL to 60 mg/mL, 30 mg/mL to 50 mg/mL, 30 mg/mL to 40 mg/mL, 40 mg/mL to 100 mg/mL, 40 mg/mL to 90 mg/mL, 40 mg/mL to 80 mg/mL, 40 mg/mL to 70 mg/mL, 40 mg/mL to 60 mg/mL, 40 mg/mL to 50 mg/mL, 50 mg/mL to 100 mg/mL, 50 mg/mL to 90 mg/mL, 50 mg/mL to 80 mg/mL, 50 mg/mL to 70 mg/mL, 50 mg/mL to 60 mg/mL, 60 mg/mL to 100 mg/mL, 60 mg/mL to 90 mg/mL, 60 mg/mL to 80 mg/mL, 60 mg/mL to 70 mg/mL, 70 mg/mL to 100 mg/mL, 70 mg/mL to 90 mg/mL, 70 mg/mL to 80 mg/mL, 80 mg/mL to 100 mg/mL, 80 mg/mL to 90 mg/mL, or 90 mg/mL to 100 mg/mL) of padsevonil. In certain preferred embodiments, a liquid pharmaceutical composition disclosed herein includes 4 mg/mL to 16 mg/mL (e.g., 4 mg/mL to 8 mg/mL or 8 mg/mL to 16 mg/mL) of padsevonil.

Atorvastatin

Additionally or alternatively, a liquid pharmaceutical composition disclosed herein may further include, e.g., atorvastatin or a pharmaceutically acceptable salt thereof. For example, a liquid pharmaceutical composition disclosed herein may include 0.1 mg/mL to 80 mg/mL (e.g., 0.1 mg/mL to 70 mg/mL, 0.1 mg/mL to 60 mg/mL, 0.1 mg/mL to 50 mg/mL, 0.1 mg/mL to 40 mg/mL, 0.1 mg/mL to 30 mg/mL, 0.1 mg/mL to 20 mg/mL, 0.1 mg/mL to 15 mg/mL, 0.1 mg/mL to 13 mg/mL, 0.1 mg/mL to 12 mg/mL, 0.1 mg/mL to 11 mg/mL, 0.1 mg/mL to 10 mg/mL, 0.1 mg/mL to 9 mg/mL, 0.1 mg/mL to 8 mg/mL, 0.1 mg/mL to 7 mg/mL, 0.1 mg/mL to 6 mg/mL, 0.1 mg/mL to 5 mg/mL, 0.1 mg/mL to 4 mg/mL, 0.1 mg/mL to 3 mg/mL, 0.1 mg/mL to 2 mg/mL, 0.1 mg/mL to 1 mg/mL, 0.1 mg/mL to 0.8 mg/mL, 0.1 mg/mL to 0.5 mg/mL, 0.1 mg/mL to 0.4 mg/mL, 0.1 mg/mL to 0.3 mg/mL, 0.1 mg/mL to 0.2 mg/mL, 0.2 mg/mL to 80 mg/mL, 0.2 mg/mL to 70 mg/mL, 0.2 mg/mL to 60 mg/mL, 0.2 mg/mL to 50 mg/mL, 0.2 mg/mL to 40 mg/mL, 0.2 mg/mL to 30 mg/mL, 0.2 mg/mL to 20 mg/mL, 0.2 mg/mL to 15 mg/mL, 0.2 mg/mL to 13 mg/mL, 0.2 mg/mL to 12 mg/mL, 0.2 mg/mL to 11 mg/mL, 0.2 mg/mL to 10 mg/mL, 0.2 mg/mL to 9 mg/mL, 0.2 mg/mL to 8 mg/mL, 0.2 mg/mL to 7 mg/mL, 0.2 mg/mL to 6 mg/mL, 0.2 mg/mL to 5 mg/mL, 0.2 mg/mL to 4 mg/mL, 0.2 mg/mL to 3 mg/mL, 0.2 mg/mL to 2 mg/mL, 0.2 mg/mL to 1 mg/mL, 0.2 mg/mL to 0.8 mg/mL, 0.2 mg/mL to 0.5 mg/mL, 0.2 mg/mL to 0.4 mg/mL, 0.2 mg/mL to 0.3 mg/mL, 0.3 mg/mL to 80 mg/mL, 0.3 mg/mL to 70 mg/mL, 0.3 mg/mL to 60 mg/mL, 0.3 mg/mL to 50 mg/mL, 0.3 mg/mL to 40 mg/mL, 0.3 mg/mL to 30 mg/mL, 0.3 mg/mL to 20 mg/mL, 0.3 mg/mL to 15 mg/mL, 0.3 mg/mL to 13 mg/mL, 0.3 mg/mL to 12 mg/mL, 0.3 mg/mL to 11 mg/mL, 0.3 mg/mL to 10 mg/mL, 0.3 mg/mL to 9 mg/mL, 0.3 mg/mL to 8 mg/mL, 0.3 mg/mL to 7 mg/mL, 0.3 mg/mL to 6 mg/mL, 0.3 mg/mL to 5 mg/mL, 0.3 mg/mL to 4 mg/mL, 0.3 mg/mL to 3 mg/mL, 0.3 mg/mL to 2 mg/mL, 0.3 mg/mL to 1 mg/mL, 0.3 mg/mL to 0.8 mg/mL, 0.3 mg/mL to 0.5 mg/mL, 0.3 mg/mL to 0.4 mg/mL, 0.4 mg/mL to 80 mg/mL, 0.4 mg/mL to 70 mg/mL, 0.4 mg/mL to 60 mg/mL, 0.4 mg/mL to 50 mg/mL, 0.4 mg/mL to 40 mg/mL, 0.4 mg/mL to 30 mg/mL, 0.4 mg/mL to 20 mg/mL, 0.4 mg/mL to 15 mg/mL, 0.4 mg/mL to 13 mg/mL, 0.4 mg/mL to 12 mg/mL, 0.4 mg/mL to 11 mg/mL, 0.4 mg/mL to 10 mg/mL, 0.4 mg/mL to 9 mg/mL, 0.4 mg/mL to 8 mg/mL, 0.4 mg/mL to 7 mg/mL, 0.4 mg/mL to 6 mg/mL, 0.4 mg/mL to 5 mg/mL, 0.4 mg/mL to 4 mg/mL, 0.4 mg/mL to 3 mg/mL, 0.4 mg/mL to 2 mg/mL, 0.4 mg/mL to 1 mg/mL, 0.4 mg/mL to 0.8 mg/mL, 0.4 mg/mL to 0.5 mg/mL, 0.5 mg/mL to 80 mg/mL, 0.5 mg/mL to 70 mg/mL, 0.5 mg/mL to 60 mg/mL, 0.5 mg/mL to 50 mg/mL, 0.5 mg/mL to 40 mg/mL, 0.5 mg/mL to 30 mg/mL, 0.5 mg/mL to 20 mg/mL, 0.5 mg/mL to 15 mg/mL, 0.5 mg/mL to 13 mg/mL, 0.5 mg/mL to 12 mg/mL, 0.5 mg/mL to 11 mg/mL, 0.5 mg/mL to 10 mg/mL, 0.5 mg/mL to 9 mg/mL, 0.5 mg/mL to 8 mg/mL, 0.5 mg/mL to 7 mg/mL, 0.5 mg/mL to 6 mg/mL, 0.5 mg/mL to 5 mg/mL, 0.5 mg/mL to 4 mg/mL, 0.5 mg/mL to 3 mg/mL, 0.5 mg/mL to 2 mg/mL, 0.5 mg/mL to 1 mg/mL, 0.5 mg/mL to 0.8 mg/mL, 1 mg/mL to 80 mg/mL, 1 mg/mL to 70 mg/mL, 1 mg/mL to 60 mg/mL, 1 mg/mL to 50 mg/mL, 1 mg/mL to 40 mg/mL, 1 mg/mL to 30 mg/mL, 1 mg/mL to 20 mg/mL, 1 mg/mL to 15 mg/mL, 1 mg/mL to 13 mg/mL, 1 mg/mL to 12 mg/mL, 1 mg/mL to 11 mg/mL, 1 mg/mL to 10 mg/mL, 1 mg/mL to 9 mg/mL, 1 mg/mL to 8 mg/mL, 1 mg/mL to 7 mg/mL, 1 mg/mL to 6 mg/mL, 1 mg/mL to 5 mg/mL, 1 mg/mL to 4 mg/mL, 1 mg/mL to 3 mg/mL, 1 mg/mL to 2 mg/mL, 2 mg/mL to 80 mg/mL, 2 mg/mL to 70 mg/mL, 2 mg/mL to 60 mg/mL, 2 mg/mL to 50 mg/mL, 2 mg/mL to 40 mg/mL, 2 mg/mL to 30 mg/mL, 2 mg/mL to 20 mg/mL, 2 mg/mL to 15 mg/mL, 2 mg/mL to 13 mg/mL, 2 mg/mL to 12 mg/mL, 2 mg/mL to 11 mg/mL, 2 mg/mL to 10 mg/mL, 2 mg/mL to 9 mg/mL, 2 mg/mL to 8 mg/mL, 2 mg/mL to 7 mg/mL, 2 mg/mL to 6 mg/mL, 2 mg/mL to 5 mg/mL, 2 mg/mL to 4 mg/mL, 2 mg/mL to 3 mg/mL, 3 mg/mL to 80 mg/mL, 3 mg/mL to 70 mg/mL, 3 mg/mL to 60 mg/mL, 3 mg/mL to 50 mg/mL, 3 mg/mL to 40 mg/mL, 3 mg/mL to 30 mg/mL, 3 mg/mL to 20 mg/mL, 3 mg/mL to 15 mg/mL, 3 mg/mL to 13 mg/mL, 3 mg/mL to 12 mg/mL, 3 mg/mL to 11 mg/mL, 3 mg/mL to 10 mg/mL, 3 mg/mL to 9 mg/mL, 3 mg/mL to 8 mg/mL, 3 mg/mL to 7 mg/mL, 3 mg/mL to 6 mg/mL, 3 mg/mL to 5 mg/mL, 3 mg/mL to 4 mg/mL, 4 mg/mL to 80 mg/mL, 4 mg/mL to 70 mg/mL, 4 mg/mL to 60 mg/mL, 4 mg/mL to 50 mg/mL, 4 mg/mL to 40 mg/mL, 4 mg/mL to 30 mg/mL, 4 mg/mL to 20 mg/mL, 4 mg/mL to 15 mg/mL, 4 mg/mL to 13 mg/mL, 4 mg/mL to 12 mg/mL, 4 mg/mL to 11 mg/mL, 4 mg/mL to 10 mg/mL, 4 mg/mL to 9 mg/mL, 4 mg/mL to 8 mg/mL, 4 mg/mL to 7 mg/mL, 4 mg/mL to 6 mg/mL, 4 mg/mL to 5 mg/mL, 5 mg/mL to 80 mg/mL, 5 mg/mL to 70 mg/mL, 5 mg/mL to 60 mg/mL, 5 mg/mL to 50 mg/mL, 5 mg/mL to 40 mg/mL, 5 mg/mL to 30 mg/mL, 5 mg/mL to 20 mg/mL, 5 mg/mL to 15 mg/mL, 5 mg/mL to 13 mg/mL, 5 mg/mL to 12 mg/mL, 5 mg/mL to 11 mg/mL, 5 mg/mL to 10 mg/mL, 5 mg/mL to 9 mg/mL, 5 mg/mL to 8 mg/mL, 5 mg/mL to 7 mg/mL, 5 mg/mL to 6 mg/mL, 6 mg/mL to 80 mg/mL, 6 mg/mL to 70 mg/mL, 6 mg/mL to 60 mg/mL, 6 mg/mL to 50 mg/mL, 6 mg/mL to 40 mg/mL, 6 mg/mL to 30 mg/mL, 6 mg/mL to 20 mg/mL, 6 mg/mL to 15 mg/mL, 6 mg/mL to 13 mg/mL, 6 mg/mL to 12 mg/mL, 6 mg/mL to 11 mg/mL, 6 mg/mL to 10 mg/mL, 6 mg/mL to 9 mg/mL, 6 mg/mL to 8 mg/mL, 6 mg/mL to 7 mg/mL, 7 mg/mL to 80 mg/mL, 7 mg/mL to 70 mg/mL, 7 mg/mL to 60 mg/mL, 7 mg/mL to 50 mg/mL, 7 mg/mL to 40 mg/mL, 7 mg/mL to 30 mg/mL, 7 mg/mL to 20 mg/mL, 7 mg/mL to 15 mg/mL, 7 mg/mL to 13 mg/mL, 7 mg/mL to 12 mg/mL, 7 mg/mL to 11 mg/mL, 7 mg/mL to 10 mg/mL, 7 mg/mL to 9 mg/mL, 7 mg/mL to 8 mg/mL, 8 mg/mL to 80 mg/mL, 8 mg/mL to 70 mg/mL, 8 mg/mL to 60 mg/mL, 8 mg/mL to 50 mg/mL, 8 mg/mL to 40 mg/mL, 8 mg/mL to 30 mg/mL, 8 mg/mL to 20 mg/mL, 8 mg/mL to 15 mg/mL, 8 mg/mL to 13 mg/mL, 8 mg/mL to 12 mg/mL, 8 mg/mL to 11 mg/mL, 8 mg/mL to 10 mg/mL, 8 mg/mL to 9 mg/mL, 9 mg/mL to 80 mg/mL, 9 mg/mL to 70 mg/mL, 9 mg/mL to 60 mg/mL, 9 mg/mL to 50 mg/mL, 9 mg/mL to 40 mg/mL, 9 mg/mL to 30 mg/mL, 9 mg/mL to 20 mg/mL, 9 mg/mL to 15 mg/mL, 9 mg/mL to 13 mg/mL, 9 mg/mL to 12 mg/mL, 9 mg/mL to 11 mg/mL, 9 mg/mL to 10 mg/mL, 10 mg/mL to 80 mg/mL, 10 mg/mL to 70 mg/mL, 10 mg/mL to 60 mg/mL, 10 mg/mL to 50 mg/mL, 10 mg/mL to 40 mg/mL, 10 mg/mL to 30 mg/mL, 10 mg/mL to 20 mg/mL, 10 mg/mL to 15 mg/mL, 10 mg/mL to 13 mg/mL, 10 mg/mL to 12 mg/mL, 10 mg/mL to 11 mg/mL, 11 mg/mL to 80 mg/mL, 11 mg/mL to 70 mg/mL, 11 mg/mL to 60 mg/mL, 11 mg/mL to 50 mg/mL, 11 mg/mL to 40 mg/mL, 11 mg/mL to 30 mg/mL, 11 mg/mL to 20 mg/mL, 11 mg/mL to 15 mg/mL, 11 mg/mL to 13 mg/mL, 11 mg/mL to 12 mg/mL, 12 mg/mL to 80 mg/mL, 12 mg/mL to 70 mg/mL, 12 mg/mL to 60 mg/mL, 12 mg/mL to 50 mg/mL, 12 mg/mL to 40 mg/mL, 12 mg/mL to 30 mg/mL, 12 mg/mL to 20 mg/mL, 12 mg/mL to 15 mg/mL, 12 mg/mL to 13 mg/mL, 13 mg/mL to 80 mg/mL, 13 mg/mL to 70 mg/mL, 13 mg/mL to 60 mg/mL, 13 mg/mL to 50 mg/mL, 13 mg/mL to 40 mg/mL, 13 mg/mL to 30 mg/mL, 13 mg/mL to 20 mg/mL, 13 mg/mL to 15 mg/mL, 15 mg/mL to 80 mg/mL, 15 mg/mL to 70 mg/mL, 15 mg/mL to 60 mg/mL, 15 mg/mL to 50 mg/mL, 15 mg/mL to 40 mg/mL, 15 mg/mL to 30 mg/mL, 15 mg/mL to 20 mg/mL, 20 mg/mL to 80 mg/mL, 20 mg/mL to 70 mg/mL, 20 mg/mL to 60 mg/mL, 20 mg/mL to 50 mg/mL, 20 mg/mL to 40 mg/mL, 20 mg/mL to 30 mg/mL, 30 mg/mL to 80 mg/mL, 30 mg/mL to 70 mg/mL, 30 mg/mL to 60 mg/mL, 30 mg/mL to 50 mg/mL, 30 mg/mL to 40 mg/mL, 40 mg/mL to 80 mg/mL, 40 mg/mL to 70 mg/mL, 40 mg/mL to 60 mg/mL, 40 mg/mL to 50 mg/mL, 50 mg/mL to 80 mg/mL, 50 mg/mL to 70 mg/mL, 50 mg/mL to 60 mg/mL, 60 mg/mL to 80 mg/mL, 60 mg/mL to 70 mg/mL, or 70 mg/mL to 80 mg/mL) of atorvastatin or a pharmaceutically acceptable salt thereof. Preferred among the atorvastatin-containing liquid pharmaceutical compositions are those including atorvastatin sodium.

Preferably, a liquid pharmaceutical composition disclosed herein includes 0.1 mg/mL to 20 mg/mL (e.g., 0.1 mg/mL to 15 mg/mL, 0.1 mg/mL to 13 mg/mL, 0.1 mg/mL to 12 mg/mL, 0.1 mg/mL to 11 mg/mL, 0.1 mg/mL to 10 mg/mL, 0.1 mg/mL to 9 mg/mL, 0.1 mg/mL to 8 mg/mL, 0.1 mg/mL to 7 mg/mL, 0.1 mg/mL to 6 mg/mL, 0.1 mg/mL to 5 mg/mL, 0.1 mg/mL to 4 mg/mL, 0.1 mg/mL to 3 mg/mL, 0.1 mg/mL to 2 mg/mL, 0.1 mg/mL to 1 mg/mL, 0.1 mg/mL to 0.8 mg/mL, 0.1 mg/mL to 0.5 mg/mL, 0.1 mg/mL to 0.4 mg/mL, 0.1 mg/mL to 0.3 mg/mL, 0.1 mg/mL to 0.2 mg/mL, 0.2 mg/mL to 20 mg/mL, 0.2 mg/mL to 15 mg/mL, 0.2 mg/mL to 13 mg/mL, 0.2 mg/mL to 12 mg/mL, 0.2 mg/mL to 11 mg/mL, 0.2 mg/mL to 10 mg/mL, 0.2 mg/mL to 9 mg/mL, 0.2 mg/mL to 8 mg/mL, 0.2 mg/mL to 7 mg/mL, 0.2 mg/mL to 6 mg/mL, 0.2 mg/mL to 5 mg/mL, 0.2 mg/mL to 4 mg/mL, 0.2 mg/mL to 3 mg/mL, 0.2 mg/mL to 2 mg/mL, 0.2 mg/mL to 1 mg/mL, 0.2 mg/mL to 0.8 mg/mL, 0.2 mg/mL to 0.5 mg/mL, 0.2 mg/mL to 0.4 mg/mL, 0.2 mg/mL to 0.3 mg/mL, 0.3 mg/mL to 20 mg/mL, 0.3 mg/mL to 15 mg/mL, 0.3 mg/mL to 13 mg/mL, 0.3 mg/mL to 12 mg/mL, 0.3 mg/mL to 11 mg/mL, 0.3 mg/mL to 10 mg/mL, 0.3 mg/mL to 9 mg/mL, 0.3 mg/mL to 8 mg/mL, 0.3 mg/mL to 7 mg/mL, 0.3 mg/mL to 6 mg/mL, 0.3 mg/mL to 5 mg/mL, 0.3 mg/mL to 4 mg/mL, 0.3 mg/mL to 3 mg/mL, 0.3 mg/mL to 2 mg/mL, 0.3 mg/mL to 1 mg/mL, 0.3 mg/mL to 0.8 mg/mL, 0.3 mg/mL to 0.5 mg/mL, 0.3 mg/mL to 0.4 mg/mL, 0.4 mg/mL to 20 mg/mL, 0.4 mg/mL to 15 mg/mL, 0.4 mg/mL to 13 mg/mL, 0.4 mg/mL to 12 mg/mL, 0.4 mg/mL to 11 mg/mL, 0.4 mg/mL to 10 mg/mL, 0.4 mg/mL to 9 mg/mL, 0.4 mg/mL to 8 mg/mL, 0.4 mg/mL to 7 mg/mL, 0.4 mg/mL to 6 mg/mL, 0.4 mg/mL to 5 mg/mL, 0.4 mg/mL to 4 mg/mL, 0.4 mg/mL to 3 mg/mL, 0.4 mg/mL to 2 mg/mL, 0.4 mg/mL to 1 mg/mL, 0.4 mg/mL to 0.8 mg/mL, 0.4 mg/mL to 0.5 mg/mL, 0.5 mg/mL to 20 mg/mL, 0.5 mg/mL to 15 mg/mL, 0.5 mg/mL to 13 mg/mL, 0.5 mg/mL to 12 mg/mL, 0.5 mg/mL to 11 mg/mL, 0.5 mg/mL to 10 mg/mL, 0.5 mg/mL to 9 mg/mL, 0.5 mg/mL to 8 mg/mL, 0.5 mg/mL to 7 mg/mL, 0.5 mg/mL to 6 mg/mL, 0.5 mg/mL to 5 mg/mL, 0.5 mg/mL to 4 mg/mL, 0.5 mg/mL to 3 mg/mL, 0.5 mg/mL to 2 mg/mL, 0.5 mg/mL to 1 mg/mL, 0.5 mg/mL to 0.8 mg/mL, 1 mg/mL to 20 mg/mL, 1 mg/mL to 15 mg/mL, 1 mg/mL to 13 mg/mL, 1 mg/mL to 12 mg/mL, 1 mg/mL to 11 mg/mL, 1 mg/mL to 10 mg/mL, 1 mg/mL to 9 mg/mL, 1 mg/mL to 8 mg/mL, 1 mg/mL to 7 mg/mL, 1 mg/mL to 6 mg/mL, 1 mg/mL to 5 mg/mL, 1 mg/mL to 4 mg/mL, 1 mg/mL to 3 mg/mL, 1 mg/mL to 2 mg/mL, 2 mg/mL to 20 mg/mL, 2 mg/mL to 15 mg/mL, 2 mg/mL to 13 mg/mL, 2 mg/mL to 12 mg/mL, 2 mg/mL to 11 mg/mL, 2 mg/mL to 10 mg/mL, 2 mg/mL to 9 mg/mL, 2 mg/mL to 8 mg/mL, 2 mg/mL to 7 mg/mL, 2 mg/mL to 6 mg/mL, 2 mg/mL to 5 mg/mL, 2 mg/mL to 4 mg/mL, 2 mg/mL to 3 mg/mL, 3 mg/mL to 20 mg/mL, 3 mg/mL to 15 mg/mL, 3 mg/mL to 13 mg/mL, 3 mg/mL to 12 mg/mL, 3 mg/mL to 11 mg/mL, 3 mg/mL to 10 mg/mL, 3 mg/mL to 9 mg/mL, 3 mg/mL to 8 mg/mL, 3 mg/mL to 7 mg/mL, 3 mg/mL to 6 mg/mL, 3 mg/mL to 5 mg/mL, 3 mg/mL to 4 mg/mL, 4 mg/mL to 20 mg/mL, 4 mg/mL to 15 mg/mL, 4 mg/mL to 13 mg/mL, 4 mg/mL to 12 mg/mL, 4 mg/mL to 11 mg/mL, 4 mg/mL to 10 mg/mL, 4 mg/mL to 9 mg/mL, 4 mg/mL to 8 mg/mL, 4 mg/mL to 7 mg/mL, 4 mg/mL to 6 mg/mL, 4 mg/mL to 5 mg/mL, 5 mg/mL to 20 mg/mL, 5 mg/mL to 15 mg/mL, 5 mg/mL to 13 mg/mL, 5 mg/mL to 12 mg/mL, 5 mg/mL to 11 mg/mL, 5 mg/mL to 10 mg/mL, 5 mg/mL to 9 mg/mL, 5 mg/mL to 8 mg/mL, 5 mg/mL to 7 mg/mL, 5 mg/mL to 6 mg/mL, 6 mg/mL to 20 mg/mL, 6 mg/mL to 15 mg/mL, 6 mg/mL to 13 mg/mL, 6 mg/mL to 12 mg/mL, 6 mg/mL to 11 mg/mL, 6 mg/mL to 10 mg/mL, 6 mg/mL to 9 mg/mL, 6 mg/mL to 8 mg/mL, 6 mg/mL to 7 mg/mL, 7 mg/mL to 20 mg/mL, 7 mg/mL to 15 mg/mL, 7 mg/mL to 13 mg/mL, 7 mg/mL to 12 mg/mL, 7 mg/mL to 11 mg/mL, 7 mg/mL to 10 mg/mL, 7 mg/mL to 9 mg/mL, 7 mg/mL to 8 mg/mL, 8 mg/mL to 20 mg/mL, 8 mg/mL to 15 mg/mL, 8 mg/mL to 13 mg/mL, 8 mg/mL to 12 mg/mL, 8 mg/mL to 11 mg/mL, 8 mg/mL to 10 mg/mL, 8 mg/mL to 9 mg/mL, 9 mg/mL to 20 mg/mL, 9 mg/mL to 15 mg/mL, 9 mg/mL to 13 mg/mL, 9 mg/mL to 12 mg/mL, 9 mg/mL to 11 mg/mL, 9 mg/mL to 10 mg/mL, 10 mg/mL to 20 mg/mL, 10 mg/mL to 15 mg/mL, 10 mg/mL to 13 mg/mL, 10 mg/mL to 12 mg/mL, 10 mg/mL to 11 mg/mL, 11 mg/mL to 20 mg/mL, 11 mg/mL to 15 mg/mL, 11 mg/mL to 13 mg/mL, 11 mg/mL to 12 mg/mL, 12 mg/mL to 20 mg/mL, 12 mg/mL to 15 mg/mL, 12 mg/mL to 13 mg/mL, 13 mg/mL to 20 mg/mL, 13 mg/mL to 15 mg/mL, or 15 mg/mL to 20 mg/mL) of atorvastatin or a pharmaceutically acceptable salt thereof.

More preferably, a liquid pharmaceutical composition disclosed herein includes 0.1 mg/mL to 16 mg/mL (e.g., 0.1 mg/mL to 15 mg/mL, 0.1 mg/mL to 13 mg/mL, 0.1 mg/mL to 12 mg/mL, 0.1 mg/mL to 11 mg/mL, 0.1 mg/mL to 10 mg/mL, 0.1 mg/mL to 9 mg/mL, 0.1 mg/mL to 8 mg/mL, 0.1 mg/mL to 7 mg/mL, 0.1 mg/mL to 6 mg/mL, 0.1 mg/mL to 5 mg/mL, 0.1 mg/mL to 4 mg/mL, 0.1 mg/mL to 3 mg/mL, 0.1 mg/mL to 2 mg/mL, 0.1 mg/mL to 1 mg/mL, 0.1 mg/mL to 0.8 mg/mL, 0.1 mg/mL to 0.5 mg/mL, 0.1 mg/mL to 0.4 mg/mL, 0.1 mg/mL to 0.3 mg/mL, 0.1 mg/mL to 0.2 mg/mL, 0.2 mg/mL to 16 mg/mL, 0.2 mg/mL to 15 mg/mL, 0.2 mg/mL to 13 mg/mL, 0.2 mg/mL to 12 mg/mL, 0.2 mg/mL to 11 mg/mL, 0.2 mg/mL to 10 mg/mL, 0.2 mg/mL to 9 mg/mL, 0.2 mg/mL to 8 mg/mL, 0.2 mg/mL to 7 mg/mL, 0.2 mg/mL to 6 mg/mL, 0.2 mg/mL to 5 mg/mL, 0.2 mg/mL to 4 mg/mL, 0.2 mg/mL to 3 mg/mL, 0.2 mg/mL to 2 mg/mL, 0.2 mg/mL to 1 mg/mL, 0.2 mg/mL to 0.8 mg/mL, 0.2 mg/mL to 0.5 mg/mL, 0.2 mg/mL to 0.4 mg/mL, 0.2 mg/mL to 0.3 mg/mL, 0.3 mg/mL to 16 mg/mL, 0.3 mg/mL to 15 mg/mL, 0.3 mg/mL to 13 mg/mL, 0.3 mg/mL to 12 mg/mL, 0.3 mg/mL to 11 mg/mL, 0.3 mg/mL to 10 mg/mL, 0.3 mg/mL to 9 mg/mL, 0.3 mg/mL to 8 mg/mL, 0.3 mg/mL to 7 mg/mL, 0.3 mg/mL to 6 mg/mL, 0.3 mg/mL to 5 mg/mL, 0.3 mg/mL to 4 mg/mL, 0.3 mg/mL to 3 mg/mL, 0.3 mg/mL to 2 mg/mL, 0.3 mg/mL to 1 mg/mL, 0.3 mg/mL to 0.8 mg/mL, 0.3 mg/mL to 0.5 mg/mL, 0.3 mg/mL to 0.4 mg/mL, 0.4 mg/mL to 16 mg/mL, 0.4 mg/mL to 15 mg/mL, 0.4 mg/mL to 13 mg/mL, 0.4 mg/mL to 12 mg/mL, 0.4 mg/mL to 11 mg/mL, 0.4 mg/mL to 10 mg/mL, 0.4 mg/mL to 9 mg/mL, 0.4 mg/mL to 8 mg/mL, 0.4 mg/mL to 7 mg/mL, 0.4 mg/mL to 6 mg/mL, 0.4 mg/mL to 5 mg/mL, 0.4 mg/mL to 4 mg/mL, 0.4 mg/mL to 3 mg/mL, 0.4 mg/mL to 2 mg/mL, 0.4 mg/mL to 1 mg/mL, 0.4 mg/mL to 0.8 mg/mL, 0.4 mg/mL to 0.5 mg/mL, 0.5 mg/mL to 16 mg/mL, 0.5 mg/mL to 15 mg/mL, 0.5 mg/mL to 13 mg/mL, 0.5 mg/mL to 12 mg/mL, 0.5 mg/mL to 11 mg/mL, 0.5 mg/mL to 10 mg/mL, 0.5 mg/mL to 9 mg/mL, 0.5 mg/mL to 8 mg/mL, 0.5 mg/mL to 7 mg/mL, 0.5 mg/mL to 6 mg/mL, 0.5 mg/mL to 5 mg/mL, 0.5 mg/mL to 4 mg/mL, 0.5 mg/mL to 3 mg/mL, 0.5 mg/mL to 2 mg/mL, 0.5 mg/mL to 1 mg/mL, 0.5 mg/mL to 0.8 mg/mL, 1 mg/mL to 16 mg/mL, 1 mg/mL to 15 mg/mL, 1 mg/mL to 13 mg/mL, 1 mg/mL to 12 mg/mL, 1 mg/mL to 11 mg/mL, 1 mg/mL to 10 mg/mL, 1 mg/mL to 9 mg/mL, 1 mg/mL to 8 mg/mL, 1 mg/mL to 7 mg/mL, 1 mg/mL to 6 mg/mL, 1 mg/mL to 5 mg/mL, 1 mg/mL to 4 mg/mL, 1 mg/mL to 3 mg/mL, 1 mg/mL to 2 mg/mL, 2 mg/mL to 16 mg/mL, 2 mg/mL to 15 mg/mL, 2 mg/mL to 13 mg/mL, 2 mg/mL to 12 mg/mL, 2 mg/mL to 11 mg/mL, 2 mg/mL to 10 mg/mL, 2 mg/mL to 9 mg/mL, 2 mg/mL to 8 mg/mL, 2 mg/mL to 7 mg/mL, 2 mg/mL to 6 mg/mL, 2 mg/mL to 5 mg/mL, 2 mg/mL to 4 mg/mL, 2 mg/mL to 3 mg/mL, 3 mg/mL to 16 mg/mL, 3 mg/mL to 15 mg/mL, 3 mg/mL to 13 mg/mL, 3 mg/mL to 12 mg/mL, 3 mg/mL to 11 mg/mL, 3 mg/mL to 10 mg/mL, 3 mg/mL to 9 mg/mL, 3 mg/mL to 8 mg/mL, 3 mg/mL to 7 mg/mL, 3 mg/mL to 6 mg/mL, 3 mg/mL to 5 mg/mL, 3 mg/mL to 4 mg/mL, 4 mg/mL to 16 mg/mL, 4 mg/mL to 15 mg/mL, 4 mg/mL to 13 mg/mL, 4 mg/mL to 12 mg/mL, 4 mg/mL to 11 mg/mL, 4 mg/mL to 10 mg/mL, 4 mg/mL to 9 mg/mL, 4 mg/mL to 8 mg/mL, 4 mg/mL to 7 mg/mL, 4 mg/mL to 6 mg/mL, 4 mg/mL to 5 mg/mL, 5 mg/mL to 16 mg/mL, 5 mg/mL to 15 mg/mL, 5 mg/mL to 13 mg/mL, 5 mg/mL to 12 mg/mL, 5 mg/mL to 11 mg/mL, 5 mg/mL to 10 mg/mL, 5 mg/mL to 9 mg/mL, 5 mg/mL to 8 mg/mL, 5 mg/mL to 7 mg/mL, 5 mg/mL to 6 mg/mL, 6 mg/mL to 16 mg/mL, 6 mg/mL to 15 mg/mL, 6 mg/mL to 13 mg/mL, 6 mg/mL to 12 mg/mL, 6 mg/mL to 11 mg/mL, 6 mg/mL to 10 mg/mL, 6 mg/mL to 9 mg/mL, 6 mg/mL to 8 mg/mL, 6 mg/mL to 7 mg/mL, 7 mg/mL to 16 mg/mL, 7 mg/mL to 15 mg/mL, 7 mg/mL to 13 mg/mL, 7 mg/mL to 12 mg/mL, 7 mg/mL to 11 mg/mL, 7 mg/mL to 10 mg/mL, 7 mg/mL to 9 mg/mL, 7 mg/mL to 8 mg/mL, 8 mg/mL to 16 mg/mL, 8 mg/mL to 15 mg/mL, 8 mg/mL to 13 mg/mL, 8 mg/mL to 12 mg/mL, 8 mg/mL to 11 mg/mL, 8 mg/mL to 10 mg/mL, 8 mg/mL to 9 mg/mL, 9 mg/mL to 16 mg/mL, 9 mg/mL to 15 mg/mL, 9 mg/mL to 13 mg/mL, 9 mg/mL to 12 mg/mL, 9 mg/mL to 11 mg/mL, 9 mg/mL to 10 mg/mL, 10 mg/mL to 16 mg/mL, 10 mg/mL to 15 mg/mL, 10 mg/mL to 13 mg/mL, 10 mg/mL to 12 mg/mL, 10 mg/mL to 11 mg/mL, 11 mg/mL to 16 mg/mL, 11 mg/mL to 15 mg/mL, 11 mg/mL to 13 mg/mL, 11 mg/mL to 12 mg/mL, 12 mg/mL to 16 mg/mL, 12 mg/mL to 15 mg/mL, 12 mg/mL to 13 mg/mL, 13 mg/mL to 16 mg/mL, 13 mg/mL to 15 mg/mL, or 15 mg/mL to 16 mg/mL) of atorvastatin or a pharmaceutically acceptable salt thereof.

Yet more preferably, a liquid pharmaceutical composition disclosed herein includes 0.2 mg/mL to 16 mg/mL (e.g., 0.2 mg/mL to 15 mg/mL, 0.2 mg/mL to 13 mg/mL, 0.2 mg/mL to 12 mg/mL, 0.2 mg/mL to 11 mg/mL, 0.2 mg/mL to 10 mg/mL, 0.2 mg/mL to 9 mg/mL, 0.2 mg/mL to 8 mg/mL, 0.2 mg/mL to 7 mg/mL, 0.2 mg/mL to 6 mg/mL, 0.2 mg/mL to 5 mg/mL, 0.2 mg/mL to 4 mg/mL, 0.2 mg/mL to 3 mg/mL, 0.2 mg/mL to 2 mg/mL, 0.2 mg/mL to 1 mg/mL, 0.2 mg/mL to 0.8 mg/mL, 0.2 mg/mL to 0.5 mg/mL, 0.2 mg/mL to 0.4 mg/mL, 0.2 mg/mL to 0.3 mg/mL, 0.3 mg/mL to 16 mg/mL, 0.3 mg/mL to 15 mg/mL, 0.3 mg/mL to 13 mg/mL, 0.3 mg/mL to 12 mg/mL, 0.3 mg/mL to 11 mg/mL, 0.3 mg/mL to 10 mg/mL, 0.3 mg/mL to 9 mg/mL, 0.3 mg/mL to 8 mg/mL, 0.3 mg/mL to 7 mg/mL, 0.3 mg/mL to 6 mg/mL, 0.3 mg/mL to 5 mg/mL, 0.3 mg/mL to 4 mg/mL, 0.3 mg/mL to 3 mg/mL, 0.3 mg/mL to 2 mg/mL, 0.3 mg/mL to 1 mg/mL, 0.3 mg/mL to 0.8 mg/mL, 0.3 mg/mL to 0.5 mg/mL, 0.3 mg/mL to 0.4 mg/mL, 0.4 mg/mL to 16 mg/mL, 0.4 mg/mL to 15 mg/mL, 0.4 mg/mL to 13 mg/mL, 0.4 mg/mL to 12 mg/mL, 0.4 mg/mL to 11 mg/mL, 0.4 mg/mL to 10 mg/mL, 0.4 mg/mL to 9 mg/mL, 0.4 mg/mL to 8 mg/mL, 0.4 mg/mL to 7 mg/mL, 0.4 mg/mL to 6 mg/mL, 0.4 mg/mL to 5 mg/mL, 0.4 mg/mL to 4 mg/mL, 0.4 mg/mL to 3 mg/mL, 0.4 mg/mL to 2 mg/mL, 0.4 mg/mL to 1 mg/mL, 0.4 mg/mL to 0.8 mg/mL, 0.4 mg/mL to 0.5 mg/mL, 0.5 mg/mL to 16 mg/mL, 0.5 mg/mL to 15 mg/mL, 0.5 mg/mL to 13 mg/mL, 0.5 mg/mL to 12 mg/mL, 0.5 mg/mL to 11 mg/mL, 0.5 mg/mL to 10 mg/mL, 0.5 mg/mL to 9 mg/mL, 0.5 mg/mL to 8 mg/mL, 0.5 mg/mL to 7 mg/mL, 0.5 mg/mL to 6 mg/mL, 0.5 mg/mL to 5 mg/mL, 0.5 mg/mL to 4 mg/mL, 0.5 mg/mL to 3 mg/mL, 0.5 mg/mL to 2 mg/mL, 0.5 mg/mL to 1 mg/mL, 0.5 mg/mL to 0.8 mg/mL, 1 mg/mL to 16 mg/mL, 1 mg/mL to 15 mg/mL, 1 mg/mL to 13 mg/mL, 1 mg/mL to 12 mg/mL, 1 mg/mL to 11 mg/mL, 1 mg/mL to 10 mg/mL, 1 mg/mL to 9 mg/mL, 1 mg/mL to 8 mg/mL, 1 mg/mL to 7 mg/mL, 1 mg/mL to 6 mg/mL, 1 mg/mL to 5 mg/mL, 1 mg/mL to 4 mg/mL, 1 mg/mL to 3 mg/mL, 1 mg/mL to 2 mg/mL, 2 mg/mL to 16 mg/mL, 2 mg/mL to 15 mg/mL, 2 mg/mL to 13 mg/mL, 2 mg/mL to 12 mg/mL, 2 mg/mL to 11 mg/mL, 2 mg/mL to 10 mg/mL, 2 mg/mL to 9 mg/mL, 2 mg/mL to 8 mg/mL, 2 mg/mL to 7 mg/mL, 2 mg/mL to 6 mg/mL, 2 mg/mL to 5 mg/mL, 2 mg/mL to 4 mg/mL, 2 mg/mL to 3 mg/mL, 3 mg/mL to 16 mg/mL, 3 mg/mL to 15 mg/mL, 3 mg/mL to 13 mg/mL, 3 mg/mL to 12 mg/mL, 3 mg/mL to 11 mg/mL, 3 mg/mL to 10 mg/mL, 3 mg/mL to 9 mg/mL, 3 mg/mL to 8 mg/mL, 3 mg/mL to 7 mg/mL, 3 mg/mL to 6 mg/mL, 3 mg/mL to 5 mg/mL, 3 mg/mL to 4 mg/mL, 4 mg/mL to 16 mg/mL, 4 mg/mL to 15 mg/mL, 4 mg/mL to 13 mg/mL, 4 mg/mL to 12 mg/mL, 4 mg/mL to 11 mg/mL, 4 mg/mL to 10 mg/mL, 4 mg/mL to 9 mg/mL, 4 mg/mL to 8 mg/mL, 4 mg/mL to 7 mg/mL, 4 mg/mL to 6 mg/mL, 4 mg/mL to 5 mg/mL, 5 mg/mL to 16 mg/mL, 5 mg/mL to 15 mg/mL, 5 mg/mL to 13 mg/mL, 5 mg/mL to 12 mg/mL, 5 mg/mL to 11 mg/mL, 5 mg/mL to 10 mg/mL, 5 mg/mL to 9 mg/mL, 5 mg/mL to 8 mg/mL, 5 mg/mL to 7 mg/mL, 5 mg/mL to 6 mg/mL, 6 mg/mL to 16 mg/mL, 6 mg/mL to 15 mg/mL, 6 mg/mL to 13 mg/mL, 6 mg/mL to 12 mg/mL, 6 mg/mL to 11 mg/mL, 6 mg/mL to 10 mg/mL, 6 mg/mL to 9 mg/mL, 6 mg/mL to 8 mg/mL, 6 mg/mL to 7 mg/mL, 7 mg/mL to 16 mg/mL, 7 mg/mL to 15 mg/mL, 7 mg/mL to 13 mg/mL, 7 mg/mL to 12 mg/mL, 7 mg/mL to 11 mg/mL, 7 mg/mL to 10 mg/mL, 7 mg/mL to 9 mg/mL, 7 mg/mL to 8 mg/mL, 8 mg/mL to 16 mg/mL, 8 mg/mL to 15 mg/mL, 8 mg/mL to 13 mg/mL, 8 mg/mL to 12 mg/mL, 8 mg/mL to 11 mg/mL, 8 mg/mL to 10 mg/mL, 8 mg/mL to 9 mg/mL, 9 mg/mL to 16 mg/mL, 9 mg/mL to 15 mg/mL, 9 mg/mL to 13 mg/mL, 9 mg/mL to 12 mg/mL, 9 mg/mL to 11 mg/mL, 9 mg/mL to 10 mg/mL, 10 mg/mL to 16 mg/mL, 10 mg/mL to 15 mg/mL, 10 mg/mL to 13 mg/mL, 10 mg/mL to 12 mg/mL, 10 mg/mL to 11 mg/mL, 11 mg/mL to 16 mg/mL, 11 mg/mL to 15 mg/mL, 11 mg/mL to 13 mg/mL, 11 mg/mL to 12 mg/mL, 12 mg/mL to 16 mg/mL, 12 mg/mL to 15 mg/mL, 12 mg/mL to 13 mg/mL, 13 mg/mL to 16 mg/mL, 13 mg/mL to 15 mg/mL, or 15 mg/mL to 16 mg/mL) of atorvastatin or a pharmaceutically acceptable salt thereof.

Still more preferably, a liquid pharmaceutical composition disclosed herein includes 0.4 mg/mL to 2 mg/mL (e.g., 0.4 mg/mL to 1.6 mg/mL, 0.4 mg/mL to 1 mg/mL, 0.4 mg/mL to 0.8 mg/mL, 0.4 mg/mL to 0.5 mg/mL, 0.5 mg/mL to 2 mg/mL, 0.5 mg/mL to 1.6 mg/mL, 0.5 mg/mL to 1 mg/mL, 0.5 mg/mL to 0.8 mg/mL, 1 mg/mL to 2 mg/mL, 1 mg/mL to 1.6 mg/mL, or 1.6 mg/mL to 2 mg/mL) of atorvastatin or a pharmaceutically acceptable salt thereof. Particularly preferred among liquid pharmaceutical compositions containing atorvastatin or a pharmaceutically acceptable salt thereof are those compositions include 1.2 mg/mL to 2.0 mg/mL. In alternative particularly preferred embodiments (e.g., for liquid pharmaceutical compositions configured for administration at a higher dosage volume (e.g., 40 mL to 50 mL)), a liquid pharmaceutical composition disclosed herein includes 0.6 mg/mL to 1.0 mg/mL of atorvastatin or a pharmaceutically acceptable salt thereof.

Excipients

Liquid pharmaceutical compositions disclosed herein may further include one or more pharmaceutically acceptable excipients, e.g., an antioxidant, an emulsifier, a tonicity agent, an acidulant, a calcium scavenging agent, or a combination thereof. Other pharmaceutically acceptable excipients can be colorants, flavoring agents, sweeteners, taste masking agent, thickening agents, and the like.

Antioxidants are pharmaceutically acceptable excipients, typically utilized for their capability to reduce oxidation-related decomposition of a pharmaceutical composition ingredient. Non-limiting examples of antioxidants include citric acid, α-tocopherol (e.g., D,L-α-tocopherol), monothioglycerol, ascorbic acid, butylated hydroxytoluene (BHT), butylated hydroxyanisole (BHA), sodium sulfite, sodium bisulfite, sodium thiosulfate, p-amino benzoic acid, glutathione, propyl gallate, and combinations thereof. Liquid pharmaceutical compositions disclosed herein may include, e.g., 0.001% (w/v) to 1% (w/v) (e.g., 0.01% (w/v) to 0.5% (w/v)) of an antioxidant.

Emulsifiers are pharmaceutically acceptable excipients that may be used to stabilize pharmaceutical compositions, e.g., against mechanical stresses like agitation, shearing and/or crystallization. Non-limiting examples of pharmaceutically acceptable emulsifiers include poloxamers (e.g., low molecular weight poloxamers (e.g., poloxamers having an average molecular weight of less than 10 kDa, e.g., poloxamer 188), polysorbates, polyoxyethylene alkyl ethers (Brij), alkylphenylpolyoxyethylene ethers (Triton-X), sodium dodecyl sulphate (SDS), polyvinylpyrrolidone (PVP), 1,2-propylene glycol, cremophor EL, cremophor RH40, lecithin, tert-butanol, ethanol, or polyoxyethylene stearate. Preferred emulsifiers are polysorbates. For example, polysorbate 80 may be used in intravenous liquid pharmaceutical compositions (e.g., solutions) at concentrations, e.g., 0.0001% (w/v) to 0.5% (w/v). Liquid pharmaceutical compositions disclosed herein may include, e.g., 0.0001% (w/v) to 5% (w/v) (e.g., 0.001% (w/v) to 4% (w/v), 0.001% (w/v) to 3% (w/v), 0.001% (w/v) to 2% (w/v), 0.001% (w/v) to 1% (w/v), 0.001% (w/v) to 0.5% (w/v), 0.001% (w/v) to 0.3% (w/v), 0.001% (w/v) to 0.1% (w/v), 0.003% (w/v) to 5% (w/v), 0.003% (w/v) to 4% (w/v), 0.003% (w/v) to 3% (w/v), 0.003% (w/v) to 2% (w/v), 0.003% (w/v) to 1% (w/v), 0.003% (w/v) to 0.5% (w/v), 0.003% (w/v) to 0.3% (w/v), 0.003% (w/v) to 0.1% (w/v), 0.1% (w/v) to 5% (w/v), 0.1% (w/v) to 4% (w/v), 0.1% (w/v) to 3% (w/v), 0.1% (w/v) to 2% (w/v), 0.1% (w/v) to 1% (w/v), 0.1% (w/v) to 0.5% (w/v), 0.1% (w/v) to 0.3% (w/v), 0.3% (w/v) to 5% (w/v), 0.3% (w/v) to 4% (w/v), 0.3% (w/v) to 3% (w/v), 0.3% (w/v) to 2% (w/v), 0.3% (w/v) to 1% (w/v), 0.3% (w/v) to 0.5% (w/v), 0.5% (w/v) to 5% (w/v), 0.5% (w/v) to 4% (w/v), 0.5% (w/v) to 3% (w/v), 0.5% (w/v) to 2% (w/v), 0.5% (w/v) to 1% (w/v), 1% (w/v) to 5% (w/v), 1% (w/v) to 4% (w/v), 1% (w/v) to 3% (w/v), 1% (w/v) to 2% (w/v), 2% (w/v) to 5% (w/v), 2% (w/v) to 4% (w/v), 2% (w/v) to 3% (w/v), 3% (w/v) to 5% (w/v), 3% (w/v) to 4% (w/v), or 4% (w/v) to 5% (w/v)) of an emulsifier. Alternatively, liquid pharmaceutical composition disclosed herein (e.g., those including propylene glycol, tert-butanol, ethanol, or a combination thereof as an emulsifier) may include, e.g., 1% (w/v) to 40% (w/v) (e.g., 1% (w/v) to 15% (w/v)) of an emulsifier. liquid pharmaceutical composition disclosed herein (e.g., those including ethanol as an emulsifier) may include, e.g., 1% (w/v) to 15% (w/v) of an emulsifier. Emulsifiers are especially advantageous for liquid pharmaceutical compositions including a pharmaceutically acceptable salt of atorvastatin, as certain pharmaceutically acceptable salts of atorvastatin can precipitate from its aqueous solution upon standing.

Tonicity agents are pharmaceutically acceptable excipients, typically utilized to modulate the tonicity of a liquid pharmaceutical composition. Tonicity in general relates to the osmotic pressure of a solution and is typically assessed relative to that of human blood serum. A liquid pharmaceutical composition disclosed herein can be hypotonic, isotonic, or hypertonic. A liquid pharmaceutical composition is preferably isotonic. An isotonic liquid pharmaceutical composition is a liquid having the same tonicity as a reference solution, e.g., an isotonic saline or the blood serum. Non-limiting examples of tonicity agents include pharmaceutically acceptable salts (e.g., alkaline salts, e.g., alkaline halides (e.g., sodium chloride and/or potassium chloride), amino acids, and sugars. Preferred tonicity agents are sodium chloride, trehalose, sucrose, and arginine. Non-limiting examples of amino acid tonicity agents include arginine, glycine, ornithine, lysine, histidine, glutamic acid, aspartic acid, isoleucine, leucine, alanine, phenylalanine, tyrosine, tryptophan, methionine, serine, and proline.

Acidulants are pharmaceutically acceptable excipients, typically utilized to modulate the pH of a liquid pharmaceutical composition. A liquid pharmaceutical composition may include sufficient amount of an acidulant to have a pH 5.5 to 8.8. The preferred pH range is 6.5 to 8.5. Typical acidulants included in liquid pharmaceutical compositions of the invention are weak Brønsted acids, e.g., acids having a pKa of 3-8. Non-limiting examples of acidulants include acetic acid, maleic acid, ascorbic acid, lactic acid, malic acid, and phosphoric acid.

Calcium-scavenging agents are chelators capable of binding calcium ions. Non-limiting examples of calcium-scavenging agents include, e.g., ethylenediaminetetraacetic acid (EDTA); ethylene glycol-bis(β-aminoethyl ether)-N,N,N′,N′-tetraacetic acid (also known as egtazic acid or EGTA), 1,2-bis(o-aminophenoxy)ethane-N,N,N′,N′-tetraacetic acid (BAPTA), and alkaline (e.g., sodium or potassium) salts thereof. Certain calcium-scavenging agents, e.g., free-acid forms of EDTA, EGTA, or BAPTA, are acidic and, therefore, can serve as acidulants. The calcium-scavenging agents may be used at concentrations of, e.g., 0.0001% (w/v) to 0.5% (w/v). Liquid pharmaceutical compositions disclosed herein may include, e.g., 0.0001% (w/v) to 5% (w/v) (e.g., 0.001% (w/v) to 4% (w/v), 0.001% (w/v) to 3% (w/v), 0.001% (w/v) to 2% (w/v), 0.001% (w/v) to 1% (w/v), 0.001% (w/v) to 0.5% (w/v), 0.001% (w/v) to 0.3% (w/v), 0.001% (w/v) to 0.1% (w/v), 0.003% (w/v) to 5% (w/v), 0.003% (w/v) to 4% (w/v), 0.003% (w/v) to 3% (w/v), 0.003% (w/v) to 2% (w/v), 0.003% (w/v) to 1% (w/v), 0.003% (w/v) to 0.5% (w/v), 0.003% (w/v) to 0.3% (w/v), 0.003% (w/v) to 0.1% (w/v), 0.1% (w/v) to 5% (w/v), 0.1% (w/v) to 4% (w/v), 0.1% (w/v) to 3% (w/v), 0.1% (w/v) to 2% (w/v), 0.1% (w/v) to 1% (w/v), 0.1% (w/v) to 0.5% (w/v), 0.1% (w/v) to 0.3% (w/v), 0.3% (w/v) to 5% (w/v), 0.3% (w/v) to 4% (w/v), 0.3% (w/v) to 3% (w/v), 0.3% (w/v) to 2% (w/v), 0.3% (w/v) to 1% (w/v), 0.3% (w/v) to 0.5% (w/v), 0.5% (w/v) to 5% (w/v), 0.5% (w/v) to 4% (w/v), 0.5% (w/v) to 3% (w/v), 0.5% (w/v) to 2% (w/v), 0.5% (w/v) to 1% (w/v), 1% (w/v) to 5% (w/v), 1% (w/v) to 4% (w/v), 1% (w/v) to 3% (w/v), 1% (w/v) to 2% (w/v), 2% (w/v) to 5% (w/v), 2% (w/v) to 4% (w/v), 2% (w/v) to 3% (w/v), 3% (w/v) to 5% (w/v), 3% (w/v) to 4% (w/v), or 4% (w/v) to 5% (w/v)) of a calcium scavenging agent. Preferred liquid pharmaceutical compositions disclosed herein may include 0.005% (w/v) to 0.5% (w/v) of the calcium-scavenging agents, e.g. 0.005% (w/v) to 0.01% (w/v); 0.005% (w/v) to 0.02% (w/v); 0.005% (w/v) to 0.05% (w/v); 0.005% (w/v) to 0.1% (w/v); 0.005% (w/v) to 0.2% (w/v); 0.01% (w/v) to 0.02% (w/v); 0.01% (w/v) to 0.02% (w/v); 0.01% (w/v) to 0.05% (w/v); 0.02% (w/v) to 0.1% (w/v); 0.01% (w/v) to 0.5% (w/v); 0.05% (w/v) to 0.1% (w/v); 0.05% (w/v) to 0.2% (w/v); 0.05% (w/v) to 0.5% (w/v); 0.1% (w/v) to 0.2% (w/v); 0.1% (w/v) to 0.5% (w/v). Other preferred liquid pharmaceutical compositions disclosed herein may include 0.01% (w/v) to 0.1% (w/v) of the calcium-scavenging agents.

Advantageously, in some embodiments, the liquid pharmaceutical compositions including a calcium-scavenging agent may include reduced quantities of an emulsifier or, in some instance, no emulsifier. Reduction or elimination of the emulsifier in the compositions is advantageous because of the typically unfavorable toxicological profiles of emulsifiers.

Administration

A liquid pharmaceutical composition disclosed herein may be a liquid pharmaceutical composition for parenteral administration, e.g., intravenous, intraperitoneal, subcutaneous, intramuscular, or intrathecal mode of administration. Parenteral administration may be by continuous infusion over a predetermined period of time. Alternatively, a liquid pharmaceutical composition disclosed herein may be for oral administration.

The liquid pharmaceutical compositions, e.g., for parenteral administration, may be provided in one or more containers. Such container may be, e.g., vial, bottle, ampoule, or another pharmaceutically acceptable container for liquid pharmaceutical compositions. Alternatively, the container may be a prefilled dosing system, e.g., a prefilled syringe, a prefilled infusion bottle, or a prefilled infusion bag.

Alternatively, liquid pharmaceutical compositions disclosed herein may be prepared shortly before administration (e.g., at the point-of-use or to be used within, e.g., 24 to 48 hours of preparation). Such liquid pharmaceutical compositions may be prepared using kits disclosed herein. Typically, a kit disclosed herein includes a first container and a second container, the first container including topiramate (e.g., topiramate as the only therapeutic agent; topiramate and levetiracetam; topiramate and brivaracetam; topiramate, levetiracetam, and atorvastatin; or topiramate, brivaracetam, and atorvastatin) or pharmaceutically acceptable salts thereof, and the second container including a pharmaceutically acceptable aqueous solution of meglumine. Kits disclosed herein are configured to provide a liquid pharmaceutical composition disclosed herein.

Liquid pharmaceutical composition disclosed herein may be used in a method of treating a patient in need thereof. The method includes administering to the patient a therapeutically effective amount of a liquid pharmaceutical composition described herein. The liquid pharmaceutical composition may be prepared from a kit. Thus, the method may include combining the first container contents and the second container contents in the kit described herein to produce a liquid pharmaceutical composition, and administering a therapeutically effective amount of the liquid pharmaceutical composition to the patient.

The therapeutically effective amount may be an amount providing, e.g., 0.5 mg/kg/day to 20 mg/kg/day (e.g., 2 mg/kg/day to 15 mg/kg/day) of topiramate. For example, the therapeutically effective amount may be, e.g., an amount providing at least 40 mg/day (e.g., at least 200 mg/day) of topiramate. Additionally or alternatively, the therapeutically effective amount may be, e.g., an amount providing 1200 mg/day or less (e.g., 400 mg/day or less) of topiramate. The therapeutically effective amount may be an amount providing, e.g., 2.5 mg/kg/day to 150 mg/kg/day (e.g., 10 mg/kg/day to 75 mg/kg/day) of levetiracetam. The therapeutically effective amount may be an amount providing, e.g., 0.2 mg/kg/day to 10 mg/kg/day (e.g., 0.5 mg/kg/day to 5 mg/kg/day) of brivaracetam. Additionally or alternatively, the therapeutically effective amount may be, e.g., an amount providing 50 mg/day to 400 mg/day (e.g., 50 mg/day to 200 mg/day or 200 mg/day to 400 mg/day) of brivaracetam. The therapeutically effective amount may be an amount providing, e.g., 0.1 mg/kg/day to 2.0 mg/kg/day (e.g., 0.2 to 1.5 mg/kg/day) of atorvastatin.

The liquid pharmaceutical composition may be administered, e.g., parenterally (e.g., intravenously, subcutaneously, or intramuscularly). The liquid pharmaceutical composition may be administered, e.g., orally.

In instances involving administration of a liquid pharmaceutical composition including topiramate or a pharmaceutically acceptable salt thereof as the only therapeutic agent, the method may further include step(s) of administering (i) levetiracetam or brivaracetam and/or (ii) atorvastatin or a pharmaceutically acceptable salt thereof. Levetiracetam may be administered as the only additional therapeutic agent or in combination with atorvastatin or a pharmaceutically acceptable salt thereof. When levetiracetam is administered in combination with atorvastatin or a pharmaceutically acceptable salt thereof, the two therapeutic agents may be administered in the same pharmaceutical composition or in different pharmaceutical compositions. When levetiracetam is administered in combination with atorvastatin or a pharmaceutically acceptable salt thereof, the two therapeutic agents may be administered by the same route of administration or by different routes of administration. A pharmaceutical composition including levetiracetam may be administered by the same route of administration as the liquid pharmaceutical composition including topiramate or a pharmaceutically acceptable salt thereof. Alternatively, a pharmaceutical composition including levetiracetam may be administered by a different route of administration as the liquid pharmaceutical composition including topiramate or a pharmaceutically acceptable salt thereof. Brivaracetam may be administered as the only additional therapeutic agent or in combination with atorvastatin or a pharmaceutically acceptable salt thereof. When brivaracetam is administered in combination with atorvastatin or a pharmaceutically acceptable salt thereof, the two therapeutic agents may be administered by the same route of administration or by different routes of administration. A pharmaceutical composition including brivaracetam may be administered by the same route of administration as the liquid pharmaceutical composition including topiramate or a pharmaceutically acceptable salt thereof. Alternatively, a pharmaceutical composition including brivaracetam may be administered by a different route of administration as the liquid pharmaceutical composition including topiramate or a pharmaceutically acceptable salt thereof. A pharmaceutical composition including atorvastatin or a pharmaceutically acceptable salt thereof may be administered by the same route of administration as the liquid pharmaceutical composition including topiramate or a pharmaceutically acceptable salt thereof. Alternatively, a pharmaceutical composition including atorvastatin or a pharmaceutically acceptable salt thereof may be administered by a different route of administration as the liquid pharmaceutical composition including topiramate or a pharmaceutically acceptable salt thereof.

In instances involving administration of a liquid pharmaceutical composition including topiramate or a pharmaceutically acceptable salt thereof and levetiracetam as the only therapeutic agents, the method may further include step of administering atorvastatin or a pharmaceutically acceptable salt thereof. A pharmaceutical composition including atorvastatin or a pharmaceutically acceptable salt thereof may be administered by the same route of administration as the liquid pharmaceutical composition including topiramate or a pharmaceutically acceptable salt thereof and levetiracetam. Alternatively, a pharmaceutical composition including atorvastatin or a pharmaceutically acceptable salt thereof may be administered by a different route of administration as the liquid pharmaceutical composition including topiramate or a pharmaceutically acceptable salt thereof and levetiracetam.

In instances involving administration of a liquid pharmaceutical composition including topiramate or a pharmaceutically acceptable salt thereof and brivaracetam as the only therapeutic agents, the method may further include step of administering atorvastatin or a pharmaceutically acceptable salt thereof. A pharmaceutical composition including atorvastatin or a pharmaceutically acceptable salt thereof may be administered by the same route of administration as the liquid pharmaceutical composition including topiramate or a pharmaceutically acceptable salt thereof and brivaracetam. Alternatively, a pharmaceutical composition including atorvastatin or a pharmaceutically acceptable salt thereof may be administered by a different route of administration as the liquid pharmaceutical composition including topiramate or a pharmaceutically acceptable salt thereof and brivaracetam.

In instances involving administration of a liquid pharmaceutical composition including topiramate or a pharmaceutically acceptable salt thereof and atorvastatin or a pharmaceutically acceptable salt thereof as the only therapeutic agents, the method may further include step of administering levetiracetam or brivaracetam. A pharmaceutical composition including levetiracetam may be administered by the same route of administration as the liquid pharmaceutical composition including topiramate or a pharmaceutically acceptable salt thereof and atorvastatin or a pharmaceutically acceptable salt thereof. Alternatively, a pharmaceutical composition including levetiracetam may be administered by a different route of administration as the liquid pharmaceutical composition including topiramate or a pharmaceutically acceptable salt thereof and atorvastatin or a pharmaceutically acceptable salt thereof. A pharmaceutical composition including brivaracetam may be administered by the same route of administration as the liquid pharmaceutical composition including topiramate or a pharmaceutically acceptable salt thereof and atorvastatin or a pharmaceutically acceptable salt thereof. Alternatively, a pharmaceutical composition including brivaracetam may be administered by a different route of administration as the liquid pharmaceutical composition including topiramate or a pharmaceutically acceptable salt thereof and atorvastatin or a pharmaceutically acceptable salt thereof.

Treatment

The patient may be in need of a treatment for, e.g., epilepsy, seizures, anoxia, stroke, traumatic brain injury, brain infection, brain abscess, aneurysm, subarachnoid hemorrhage, status epilepticus, refractory status epilepticus, refractory partial onset seizures (POS), addiction (e.g., gambling addiction or drug addiction), migraines, substance dependence, alcoholism, cocaine dependence, opioid dependence, nicotine dependence, metabolic syndrome X, diabetes mellitus, type 2, vomiting, obsessive-compulsive disorder, refractory generalized social phobia, Tourette Syndrome, levodopa-induced dyskinesia in Parkinson's Disease, Prader-Willi syndrome, multiple sclerosis, Lennox-Gastaut Syndrome, Dravet's syndrome, bipolar disorder, obesity, post-traumatic stress disorder, headaches (e.g., cluster headaches or severe headaches), and conditions caused by exposure to a chemical warfare nerve agent. Preferably, the patient is in need of a treatment for post-traumatic epilepsy and post-traumatic epilepsy associated central nervous system symptoms, epilepsy, seizures, anoxia and anoxia induced brain injury, stroke, traumatic brain injury, brain infection, subarachnoid hemorrhage, brain abscess, status epilepticus, refractory status epilepticus, or refractory partial onset seizures. The patient may be in need of, e.g., neuroprotection. More preferably, the patient is in need of a treatment for traumatic brain injury, stroke, or a brain infection. The patient may be in need of a treatment for, e.g., a brain abscess.

Each of the compounds may be, for example, administered to the patient in a single dose or in multiple doses. For any such combinations, the frequency of dosing may be the same for each of the compounds to be combined, or may be individually selected for each of the individual compounds to be combined. When multiple doses are administered, the doses may be separated from one another by, for example, 1-24 hours, or 1-7 days. The compound may be administered according to a schedule or the compound may be administered without a predetermined schedule. An active compound may be administered, for example, 1, 2, 3, 4, 5, 6, 7, 8, 9, 10, 11, or 12 times per day, every 2nd, 3rd, 4th, 5th, or 6th day, or 1, 2, 3, 4, 5, 6, or 7 times per week. In the case of parenteral administration, such as intravenous or subcutaneous administration, each compound or combination may be administered as bolus administration one or several times per day (1-12 times per day), or as slow bolus with each individual bolus administration taking 1 to 120 min, or as continuous infusion, without or with loading bolus administration. It is to be understood that, for any particular subject, specific dosage regimes should be adjusted over time according to the individual need and the professional judgment of the person administering or supervising the administration of the compositions.

The time periods during which the therapeutic combination may be administered may be as described herein.

According to a preferred embodiment, the treatment is to be administered within a defined time frame after the occurrence of the traumatic brain injury. The initiation treatment with effective doses is initiated within less than 7 days after the brain insult, preferably within less than 48 or 24 h after the brain insult, and most preferably within less than 8 h after the brain insult. The initial treatment is continued for a period of 3 day to 3 months after the insult, preferably for 5-30 days. The initiation treatment may be followed by a continuation treatment with the same combination immediately thereafter, either by a different administration route or using the same administration route. Such prolonged treatment can be expected to continue after the initiation treatment for 3 to 6 months, or, if epileptogenesis is only ameliorated, may be administered chronically, to treat the remaining symptoms, as medically indicated, or until occurrence of uncontrolled seizures, which require a change in treatment regimen.

Preparation

When the excipient serves as a diluent, it can be a solid, semisolid, or liquid material (e.g., isotonic saline or aqueous meglumine), which acts as a vehicle, carrier, or medium for the therapeutic agent.

The pharmaceutical forms suitable for injectable use include sterile aqueous solutions (where water soluble) or sterile powders for the extemporaneous preparation of sterile injectable solutions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, using emulsifiers. The prevention of the action of microorganisms can be brought about by various antibacterial and antifungal agents, for example, parabens, chlorobutanol, phenol, sorbic acid, thimerosal, and the like. In many cases, it will be preferable to include a tonicity agent, for example, a sugar or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization (e.g., sterile filtration using a microfilter with a typical pore size of about 0.22 μm or less). Generally, dispersions are prepared by incorporating the various sterilized active ingredient into a sterile vehicle which contains the basic dispersion medium and the required other ingredients from those enumerated above. In the case of preparing sterile powders for the manufacture of sterile injectable solutions, the preferred methods of preparation are vacuum drying, or freeze-drying optionally together with any additional desired ingredient.

Alternative sterile preparation techniques include autoclaving or ionizing radiation sterilization (i.e., gamma-radiation sterilization) of individual ingredients and primary packaging material or the whole formulation. In case of sterilization of individual ingredients and the primary packaging material, the individual components must be handled in a way that avoids contamination, i.e. may be handled in a sterile environment to prevent contamination during handling, mixing and filling into the primary packaging material, such as the vials, bottle, or infusion bags.

It is especially advantageous to formulate parenteral compositions in unit dosage forms for ease of administration and dosage uniformity. A unit dosage form as used herein refers to physically discrete units suited for administration as unitary dosages for the mammalian subjects to be treated; each unit containing a predetermined quantity of active material calculated to produce the desired therapeutic effect in association with the required pharmaceutical carrier.

The principal active ingredients are compounded for convenient and effective administration in effective amounts with a suitable pharmaceutically acceptable carrier in a unit dosage form as hereinbefore described. A unit dosage form can, for example, be prepared using kits described herein. The dosages for some therapeutic agents described herein may be determined by reference to the usual dose and manner of administration of the said ingredients.

These pharmaceutical compositions can be manufactured using methods known in the art, e.g., by conventional mixing, dissolving, levigating, emulsifying, encapsulating, entrapping, or lyophilizing processes. Methods known in the art for making pharmaceutical compositions are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen.

The dosage of the individual compounds administered as combination treatments used in the methods described herein, or pharmaceutically acceptable salts thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the individual to be treated. One of skill in the art can determine the appropriate dosage based on the above factors. The compounds used in the methods described herein may be administered initially in a suitable dosage that may be adjusted as required, depending on the clinical response. In general, a suitable daily dose of each of the compounds in combinations will be that amount of each compound that is the lowest dose effective to produce a therapeutic effect. Such an effective dose will generally depend upon the factors described above.

A compound identified as capable of treating any of the conditions described herein if administered in the combination of compounds, using any of the methods described herein, may be administered to patients or animals as a liquid pharmaceutical composition (e.g., in unit dosage form). The chemical compounds for use in such therapies may be produced and isolated by any standard technique known to those in the field of medicinal chemistry. Conventional pharmaceutical practice may be employed to provide suitable pharmaceutical compositions to administer the identified compound to patients suffering from traumatic brain injury, brain infection, brain abscess, stroke, brain ischemia including ischemic stroke, status epilepticus, and brain tumors. The treatment is initiated after the occurrence/diagnosis of the respective insult, and aims at treating epileptogenesis. Administration may begin before the patient is symptomatic.

Exemplary routes of administration of the liquid pharmaceutical compositions include oral, intranasal, intradermal, intramuscular, parenteral, intravenous, intra-arterial, intracranial, subcutaneous, intraorbital, intraventricular, intraspinal, intrathecal, and intraperitoneal. The compounds desirably are administered with a pharmaceutically acceptable carrier. Liquid pharmaceutical compositions described herein formulated for treatment of the disorders described herein are also part of the present invention.

The liquid pharmaceutical compositions described herein include those formulated for oral administration. Oral liquid pharmaceutical compositions can be, for example, in the form of a liquid solution or suspension, which contains the active ingredient(s) in a mixture with pharmaceutically acceptable excipients, e.g., as described herein. Preferably, oral liquid pharmaceutical compositions are solutions.

The liquid pharmaceutical compositions can also include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

Principles useful in the formulation of liquid pharmaceutical compositions and kits therefor are described, e.g., in Remington: The Science and Practice of Pharmacy, 21st Ed., Gennaro, Ed., Lippincott Williams & Wilkins (2005) and in The United States Pharmacopeia: The National Formulary (USP 36 NF31), published in 2013.

The following examples are meant to illustrate the invention. They are not meant to limit the invention in any way.

### EXAMPLES

### Example 1: Preclinical Evaluation of Fixed Combinations in a Rat Model of TBI

The drug combinations described here are evaluated for antiepileptogenic or disease-modifying activity in a rat model of fluid percussion injury (FPI)-induced TBI, in which rostral parasagittal fluid percussion injury in rats reliably induces a perilesional, neocortical epileptic focus within weeks after injury.

In this model, Sprague-Dawley male rats (32-36 days old) are mechanically ventilated under halothane anesthesia, and a 3 mm-diameter craniotomy is centered 2 mm posterior to bregma, 3 mm from the midline. Animals are disconnected from the ventilator and administered an 8-millisecond pressure pulse (3.4 or 3.7 atm) through the FPI device, and ventilation is resumed after a uniform 10-second interval of post-traumatic apnea. Epidural electrodes for seizure monitoring are incorporated into acrylic headsets that are securely anchored to the skull. Five epidural electrodes are implanted using procedures designed to avoid damage to the underlying neocortex. The entire EEG recording assembly is encased in dental acrylic and adhered to the skull. Video-electrocorticographic (ECoG) monitoring is performed during and up to 3 months after termination of treatment, i.e. during the first 4 weeks, but also during the drug-free period after treatment. Drug combination treatment starts 15 min-8 hours after injury and is continued for 3-4 weeks, i.e., during the latent period before onset of epilepsy in this model. Drug-treated groups of rats are compared with vehicle (“placebo”)-treated groups, using randomized and blinded protocols.

Only drug combinations that are effective in the TBI rat model are included in clinical trials. Antiepileptogenic efficacy of drug treatment is defined by a significant difference, using stand statistical methods, in the incidence of seizure-free rats between the group of drug-treated rats vs. the group of vehicle-treated rats. EEG measurements conducted after termination of the treatment are used to evaluate the seizure activity. Disease-modifying efficacy of drug treatment is defined by a significant decrease in one or more of the following parameters: frequency, severity, or duration of spontaneous seizures in the drug-treated group vs. the vehicle-treated group. Plasma drug levels are determined in the rat study to allow drug dose selection for a clinical trial.

### Example 1a

The above described model is applied for the combination of topiramate, levetiracetam, and deferoxamine. The following doses are administered: Levetiracetam: 200 mg/kg three times daily (t.i.d.) via the intraperitoneal route (i.p.), topiramate 30 mg/kg i.p. t.i.d., and deferoxamine, 40 mg/kg i.p. t.i.d. The treatment is initiated 60 min after the fluid injury and continued for 21 days. Electrographic seizure activity is recorded at 1 and 5 weeks after termination of treatment. The selected doses represent the high dose treatment scheme as described above. The route of administration is the intraperitoneal route over the first 5 days and then the oral route for the remaining treatment period.

In addition, the same combination is tested using a low dose treatment scheme. Levetiracetam: 100 mg/kg t.i.d., topiramate 15 mg/kg t.i.d., deferoxamine, 20 mg/kg q.d.

### Example 1b

The above described model is applied for the combination of topiramate, levetiracetam, and atorvastatin. The following doses are administered: Levetiracetam: 200 mg/kg i.p. t.i.d., topiramate 30 mg/kg i.p. t.i.d., and atorvastatin, 10 mg/kg i.p. or p.o., t.i.d. The treatment is initiated 60 min after the fluid injury and is continued for 21 days. Electrographic seizure activity is recorded at 1 and 5 weeks after termination of treatment. The selected doses represent the high dose treatment scheme as described above. The route of administration is the intraperitoneal route.

In addition, the same combination is tested using a low dose treatment scheme. Levetiracetam: 100 mg/kg t.i.d., topiramate 15 mg/kg t.i.d., atorvastatin, 5 mg/kg t.i.d.

The administration of a high dose regimen of 200 mg/kg levetiracetam, 30 mg/kg topiramate, and 10 mg/kg atorvastatin, was well tolerated. Following fluid percussion injury (FPI) of vehicle treated rats (15 rats injured), one rat died from the injury procedure within less than one hour (6.67% mortality). The remaining animals survived and were counted as survivor after one week and onwards.

In the treatment group, animals were exposed to FPI and treatment was started 1 h after injury, administered 3 times daily. In this group, 2 animals died from the injury within less than one hour, these deaths were unrelated to the treatment. All treated animals had normal grooming behavior and activity level. No distressed animals were observed. It could be concluded, that the combination is well tolerated. Analysis of plasma samples from the animals dosed with the combination revealed, that three-times daily dosing resulted in an exposure determined as area under the curve of the plasma level time curve in the range of the therapeutic anticonvulsant plasma exposure in man for levetiracetam and topiramate. For atorvastatin, continuous exposure could be demonstrated for 24 h. The mean plasma levels 8 h after the first dose (just prior the next dose) amounted to 26,800, 1,900, and 8.5 ng/ml for levetiracetam, topiramate, and atorvastatin, respectively.

Four weeks after FPI followed by one week of treatment, animals were evaluated for electrographic seizure frequency and total seizure duration. The treatment for one week with the triple combination resulted in a reduction in number of electrographic seizures per animal and in the total duration of seizures per animal, indicating, that the short-term treatment with the triple combination starting after the injury resulted in an anti-epileptogenic effect in a predictive animal model of traumatic brain injury.

### Example 1c

The above described model is applied for the combination of topiramate and levetiracetam. The following doses are administered: Levetiracetam: 200 mg/kg i.p. t.i.d., topiramate 30 mg/kg i.p. t.i.d. The treatment is initiated 60 min after the fluid injury and continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1d

The above described model may be applied for the combination of topiramate, levetiracetam, and ceftriaxone. The following doses are administered: Levetiracetam: 200 mg/kg i.p. t.i.d., topiramate 30 mg/kg i.p. t.i.d. and ceftriaxone 200 mg/kg i.p. t.i.d. The treatment is initiated 60 min after the fluid percussion injury and continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1e

The above described model may be applied for the combination of topiramate, levetiracetam, and gabapentin. The following doses will be administered: Levetiracetam: 200 mg/kg i.p. t.i.d., topiramate 30 mg/kg i.p. t.i.d. and gabapentin 200 mg/kg i.p. t.i.d. The treatment is initiated 60 min after the fluid percussion injury and continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose. Gabapentin in this combination may be replaced with gabapentin derivative pregabalin at the high dose of 100 mg/kg i.e. t.i.d.

### Example 1f

The above described model is applied for the combination of topiramate, levetiracetam, and pregabalin. The following doses is administered: Levetiracetam: 200 mg/kg i.p. t.i.d., topiramate 30 mg/kg i.p. t.i.d. and pregabalin 60 mg/kg i.p. t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1g

The above described model is applied for the combination of topiramate, levetiracetam, and ceftriaxone. The following doses is administered: Levetiracetam: 200 mg/kg i.p. t.i.d., topiramate 30 mg/kg i.p. t.i.d. and ceftriaxone 200 mg/kg i.p. t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1h

The above described model is applied for the combination of topiramate, levetiracetam, and α-tocopherol. The following doses is administered: Levetiracetam: 200 mg/kg i.p. t.i.d., topiramate 30 mg/kg i.p. t.i.d. and α-tocopherol, 250 mg/kg subcutaneously t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1i

The above described model is applied for the combination of levetiracetam, deferoxamine and melatonin. The following doses is administered: Levetiracetam: 200 mg/kg i.p. t.i.d., deferroxamine 40 mg/kg i.p. t.i.d. and melatonin 10 mg/kg s.c. t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1 j

The above described model is applied for the combination of levetiracetam, deferoxamine and celecoxib. The following doses is administered: Levetiracetam: 200 mg/kg i.p. t.i.d., deferroxamine 40 mg/kg i.p. t.i.d. and celecoxib 10 mg/kg s.c. t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1k

The above described model is applied for the combination levetiracetam, deferoxamine, gabapentin, and fingolimod. The following doses is administered: Levetiracetam: 200 mg/kg i.p. t.i.d., deferoxamine 40 mg/kg i.p. t.i.d. gabapentin 200 mg/k s.c., and fingolimod 1 mg/kg i.p. t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1l

The above described model is applied for the combination of levetiracetam, deferoxamine, and ceftriaxone. The following doses is administered: Levetiracetam: 200 mg/kg i.p. t.i.d., deferoxamine 40 mg/kg i.p. t.i.d. and ceftriaxone 200 mg/kg t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1m

The above described model is applied for the combination of levetiracetam and perampanel. The following doses is administered: Levetiracetam: 200 mg/kg i.p. t.i.d., and perampanel 2 mg/kg mg/kg t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1n

The above described model is applied for the combination of levetiracetam, perampanel, and ceftriaxone. The following doses is administered: Levetiracetam: 200 mg/kg i.p. t.i.d., perampanel 2 mg/kg i.p. t.i.d. and ceftriaxone 200 mg/kg t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1o

The above described model is applied for the combination of levetiracetam, parecoxib, and anakinra. The following doses is administered: Levetiracetam: 200 mg/kg i.p. t.i.d., parecoxib 1 mg/kg i.p. t.i.d. and anakinra 100 mg/kg mg/kg t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1p

The above described model is applied for the combination of levetiracetam, and phenobarbital. The following doses is administered: Levetiracetam: 200 mg/kg i.p. t.i.d., and phenobarbital initial bolus dose of 25 mg/kg i.p. followed by 15 mg/kg b.i.d. i.p. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1q

The above described model is applied for the combination of levetiracetam and agmatine. The following doses is administered: levetiracetam 200 mg/kg i.p. t.i.d. and agmatine 100 mg/kg i.p. t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1r

The above described model is applied for the combination of levetiracetam and melatonin and perampanel and atorvastatin and gabapentin. The following doses is administered: levetiracetam: 200 mg/kg i.p. t.i.d., melatonin 10 mg/kg s.c. t.i.d., perampanel 2 mg/kg i.p. t.i.d., atorvastatin, 10 mg/kg i.p. or p.o., t.i.d. and gabapentin 200 mg/kg i.p. t.i.d. Gabapentin in this combination may be replaced with the gabapentin derivative pregabalin at the high dose of 100 mg/kg i.e. t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1s

The above described model is applied for the combination of levetiracetam and N-acetyl cysteine and ceftriaxone and losartan and gabapentin. The following doses is administered: Levetiracetam: 200 mg/kg i.p. t.i.d., N-acetyl cysteine 300 mg/kg i.p. t.i.d., ceftriaxone 200 mg/kg i.p. t.i.d, losartan 10 mg/kg i.p. t.i.d., and gabapentin 200 mg/kg i.p. t.i.d. Gabapentin in this combination may be replaced with the gabapentin derivative pregabalin at the high dose of 100 mg/kg i.e. t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1t

The above described model is applied for the combination of levetiracetam and sulforaphane and perampanel and losartan and gabapentin. The following doses is administered: levetiracetam: 200 mg/kg i.p. t.i.d., sulforaphane 5 mg/kg i.p. t.i.d., perampanel 10 mg/kg i.p. t.i.d, losartan 10 mg/kg i.p. t.i.d., and gabapentin 200 mg/kg i.p. t.i.d. Gabapentin in this combination may be replaced with the gabapentin derivative pregabalin at the high dose of 100 mg/kg i.e. t.i.d. The treatment is initiated 60 min after the fluid percussion injury and is continued for 21 days. Electrographic seizure activity is recorded at 1 and 5 weeks after termination of treatment. The selected doses represent the high dose treatment scheme as described above. The route of administration is the intraperitoneal route. In addition, the same combination is tested using a low dose treatment scheme, administering 50% of the high dose regime for each dose.

### Example 1u

The above described model is applied for the combination of levetiracetam, ceftriaxone and atorvastatin, following the low dose regimen. The following doses are administered: levetiracetam (60 mg/kg t.i.d.), atorvastatin (3 mg/kg t.i.d.) and ceftriaxone (60 mg/kg t.i.d). The treatment is initiated 60 min after the fluid injury and continued for 21 days. Electrographic seizure activity is recorded at 1 and 5 weeks after termination of treatment. The selected doses represent the low dose treatment scheme. The route of administration is the intraperitoneal route. In addition, the same combination is tested using a high dose treatment scheme, i.e. 200 mg/kg levetiracetam, 10 mg/kg atorvastatin, and 200 mg/kg ceftriaxone.

### Example 1v

The above described model is applied according to the examples 1a-1t, where levetiracetam at the dose of 200 mg/kg i.p. t.i.d. is replaced with brivaracetam:10 mg/kg i.p. t.i.d or etiracetam 200 mg/kg i.p. t.i.d. In addition, the same combinations is tested using a low dose treatment scheme, administering 50% of the high dose regime for each dose and each compound.

Brivaracetam, etiracetam, and padsevonil may be used instead of levetiracetam in all examples described above, with the respective dose adjustment.

### Example 2: Evaluation of the Combination Therapies in a Controlled Cortical Impact (CCI) Model

A controlled cortical impact (CCI) injury model in young rats followed by ex-vivo evaluation of epileptogenic potentials in neocortical slices is also an appropriate model to evaluate a potential effect of a drug combination on epileptogenesis. For this model, rats are subjected to severe CCI trauma (2.0 mm depth) and then given a single dose of the triple combination of levetiracetam, 200 mg/kg, topiramate, 30 mg/kg, and atorvastatin, 10 mg/kg, immediately after injury as single dose. Control animals are also subjected to CCI injury but receive only vehicle injection at the same volume. At 2-3 weeks after injury, cortical hyperexcitability and epileptiform activity is assessed via in vitro/ex vivo electrophysiological recordings of neocortical brain slices from treated and control animals. Coronal slices (400 μm; 5 slices per rat) of somatosensory cortex are prepared from regions adjacent to the injury site and are maintained in an interface recording chamber at 31.0±1.0° C. Slices are examined for epileptiform activity via intracellular and extracellular recordings obtained from cortical layer V. Evoked responses are triggered by single, brief electrical stimuli (200 μs) applied in layer VI.

Post-injury administration of a single dose of the triple combination of levetiracetam, topiramate and atorvastatin prevented posttraumatic epilepsy in rat neocortex after CCI injury. Slices from treated rats showed a much lower incidence of evoked and spontaneous epileptiform activity than slices from sham-treated control rats.

Evoked epileptiform discharges were observed in slices from all (100%) sham-treated rats, with at least 1 slice from each animal showing epileptiform activity. Spontaneous epileptiform discharges were recorded in slices from 75% of sham-treated animals. In comparison, evoked epileptiform activity was observed in slices from significantly fewer animals and spontaneous epileptiform discharges were nearly completely suppressed in the treated animals.

In this model, the combination of levetiracetam (60 mg/kg), atorvastatin (3 mg/kg) and ceftriaxone (60 mg/kg) was also effective.

### Example 3: Preclinical Evaluation of Fixed Combinations in a Controlled Cortical Impact (CCI) Mouse Model of TBI

In addition, the drug combinations is evaluated in a controlled cortical impact (CCI) mouse model of TBI. Adult male and female mice are exposed to severe CCI injury. Mice are anesthetized using 3% isoflurane, heads shaved, and fixed in a stereotaxic frame (Kopf, Tujunga, Calif.) on a heating pad to maintain body temperature. Mice are maintained on 3% isoflurane, provided through a nose cone. A 3 mm craniotomy is performed using a portable dental drill over the right motor cortex. Bone dust is removed, then the bone flab is carefully removed without damage to the dura. The injury is generated using a 2 mm stainless steel piston attached to a CCI device (Leica Biosystems, model #39463920) at 4 m/s velocity, 1.2 mm depth, and impact duration of 300 ms. After injury the skin is sutured using vicryl sutures (Ethicon, Mokena, Ill., USA). Within a delay of 8-12 weeks 30 to 50% of all injured mice develop spontaneous generalized seizures at a rate of 1 to 3 seizures per day. Antiepileptogenic efficacy of drug treatment is defined by a significant difference in the incidence of seizure-free mice between the group of drug-treated mice vs. the group of vehicle-treated mice. Disease-modifying efficacy of drug treatment is defined by a significant decrease in frequency, severity or duration of spontaneous seizures in the drug-treated group vs. the vehicle-treated group.

This model is used to test the preferred combinations as described in Example 1a-u, and also other preferred combinations. The doses to be used in mice in these trials correspond to the doses used in the rat trials, expressed as mg/kg body weight.

### Example 4: Clinical Evaluation of Drug Combinations

The drug combinations described here (see example 1a-d) are evaluated for antiepileptogenic or disease-modifying activity in patients with traumatic brain injury (TBI) with a high risk for developing epilepsy after traumatic brain injury (post-traumatic epilepsy, PTE).

The first safety (“Phase 1”) study tests safety and tolerability of the combinations described in Patients with high (20-30%) risk for developing PTE after TBI. These are TBI patients in whom TBI results in intracranial hemorrhage(s), skull fracture(s), penetration of the brain with the object of injury (such as a bullet) or skull fragment, and who have “early” seizure after TBI. Early occurrence of seizures is defined as a seizure that occurs within seven days after injury. Such patients are treated with the drug combinations described. The drug doses chosen correspond to the high-dose regimen, as described above. Thus, levetiracetam is dosed up to 55 mg/kg to a maximum of 6,000 mg/day, topiramate at a dose of 400 mg/day, and deferoxamine at a dose of up to 62 mg/kg/day up to a maximum of 6000 mg/day. If atorvastatin is used instead of deferoxamine, the atorvastatin dose is 80 mg/day.

Treatment with the drug combinations described above commences within 8 hours of injury, as a preferred intervention time window, but may be also be delayed to 24-48 h after injury. Treatment with the drug combinations described above continues for 28 days. Medication is administered intravenously for the first 1-7 days of treatment. Next, the medication is administered intravenously, orally, nasogastrically or by other treatment routes, dependent on the consciousness and the health status of the patient. Blood levels of the drugs is checked and compared with the blood levels seen in the positive animal FPI testing. Outcome evaluations of the study may include side effects of the combinations described and blood levels of the drugs.

This phase of testing may establish safety and tolerability of the drug combinations and ascertain whether, using the human equivalent dosing, blood levels of the drugs achieved in patient with TBI are comparable with drug levels of the drugs in the successful animal preclinical FPI/PTE testing.

Based on the outcome of the safety study, the doses may be adjusted, but if tolerable, a useful dose is tested in a larger patient population in an efficacy study (pivotal “Phase 3” efficacy study). Such a Phase 3 study is one test of efficacy, safety and tolerability of the combinations described herein. Patients with a high (e.g., 20-30%) risk for developing PTE after TBI are evaluated. These are typically TBI patients in whom TBI results in intracranial hemorrhage(s), skull fracture(s), penetration of the brain with the object of injury (such as a bullet) or skull fragment, and who have “early” seizure after TBI, defined as a seizure that occurs within seven days after injury.

Such patients are treated with a drug combination described herein or with placebo. The Phase 3 study may follow standard Phase 3 design of double-blind, placebo-controlled randomized studies. Patients are randomized in a double-blinded fashion to receive treatment with either the described drug combinations or with placebo. Treatment commences within 8 hours of injury, as a preferred time window, but this time window may be extended to up to 24-48 h after injury. Treatment typically occurs over a 1-3 month time period.

Medication is administered intravenously for the first 1-7 days of treatment. Next, medication is administered intravenously, orally, nasogastrically or by other treatment routes, depending on the individual health status of the patient.

As most (e.g., 80%) of PTE starts within 2 years of injury, patients are evaluated after completion of treatment, for example, up to 2 years from an injury. Outcome evaluations of the study may include incidence of PTE 1 year and 2 years after injury, treatment side effects, treatment discontinuation because of side effects, mortality, and blood levels of the drugs. These outcomes are compared between patients treated with drug combinations described and patients treated with placebo. Standard statistical procedures shall be used to evaluate study outcomes. Drug combinations described shall be considered effective in preventing epilepsy, i.e. antiepileptogenic, if incidence of PTE at 2 years after injury is statistically lower in patients treated with drug combinations compared with patients treated with placebo or if presence of a validated biomarker of PTE is statistically lower in patients treated with drug combinations compared with patients treated with placebo.

Severity of epilepsy in those TBI patients who develop epilepsy may also be evaluated. Severity of epilepsy shall be determined by frequency of seizures per unit of time (e.g, 28 days) and by seizure type, defined with increasing severity as focal seizure without loss of awareness, focal seizure with loss of awareness, and focal seizure with loss of awareness with secondary generalized tonic clonic seizure.

Severity of epilepsy in those TBI patients who develop epilepsy are compared between TBI patients treated with drug combination and those treated with placebo using standard statistical procedures. Drug combination shall be considered to have a disease modifying property if PTE severity is statistically significantly lower in TBI patients treated with drug combinations compared with TBI patients treated with placebo.

### Example 4a

The above described model is applied for the combination of topiramate, levetiracetam, and deferoxamine. The following doses are administered: levetiracetam 55 mg/kg/day up to a maximum of 6,000 mg/person/day, topiramate 400 mg/day, and deferoxamine 62 mg/kg/day up to a maximum of 6000 mg/day. In addition, the same combination is tested using a low dose treatment scheme of levetiracetam 30 mg/kg/day, topiramate 100 mg/day, deferoxamine, 30 mg/kg/day. Treatment shall preferably start within 8 hours of injury, but could be started also 24-48 h after injury, if 8 h is deemed not feasible. Treatment shall last 1-3 months. Treatment shall be administered intravenously for the first 1-7 days of treatment. Treatment shall be administered intravenously, orally, nasogastrically or by other treatment routes after the first 1-7 days of treatment, depending on the individual health status of the patient. Patients may continue to be evaluated after completion of treatment, for total of 2 years from injury. Outcome evaluations of the study may include incidence and severity of PTE 1 year and 2 years after injury, treatment side effects, treatment discontinuation because of side effects, mortality, and blood levels of the drugs. These outcomes are compared between patients treated with drug combinations described and patients treated with placebo. Standard statistical procedures shall be used to evaluate study outcomes.

### Example 4b

The above described model is applied for the combination of topiramate, levetiracetam, and atorvastatin. The following doses are administered: levetiracetam 55 mg/kg/day up to a maximum of 6,000 mg/person/day, topiramate 400 mg/day, and atorvastatin 80 mg/day. In addition, the same combination is tested using a low dose treatment scheme of levetiracetam 30 mg/kg/day, topiramate 100 mg/day, atorvastatin 20 mg/day. Treatment shall preferably start within 8 hours of injury, but could be started also 24-48 h after injury, if 8 h is deemed not feasible. Treatment shall last 1-3 months. Treatment shall be administered intravenously for the first 1-7 days of treatment. Treatment shall be administered intravenously, orally, nasogastrically or by other treatment routes after the first 1-7 days of treatment, depending on the individual health status of the patient. Patients may continue to be evaluated after completion of treatment, for total of 2 years from injury. Outcome evaluations of the study may include incidence and severity of PTE 1 year and 2 years after injury, treatment side effects, treatment discontinuation because of side effects, mortality, and blood levels of the drugs. These outcomes are compared between patients treated with drug combinations described and patients treated with placebo. Standard statistical procedures shall be used to evaluate study outcomes.

### Example 4c

The above described model is applied for the combination of topiramate and levetiracetam, The following doses are administered: levetiracetam: 55 mg/kg/day up to a maximum of 6,000 mg/person/day, and topiramate 400 mg/day. In addition, the same combination is tested using a low dose treatment scheme. Levetiracetam: 30 mg/kg/day, and topiramate 100 mg/day. Treatment shall preferably start within 8 hours of injury, but could be started also 24-48 h after injury, if 8 h is deemed not feasible Treatment shall last 1-3 months. Treatment shall be administered intravenously for the first 1-7 days of treatment. Treatment shall be administered intravenously, orally, nasogastrically or by other treatment routes after the first 1-7 days of treatment, depending on the individual health status of the patient. Patients may continue to be evaluated after completion of treatment, for total of 2 years from injury. Outcome evaluations of the study may include incidence and severity of PTE 1 year and 2 years after injury, treatment side effects, treatment discontinuation because of side effects, mortality, and blood levels of the drugs. These outcomes are compared between patients treated with drug combinations described and patients treated with placebo. Standard statistical procedures shall be used to evaluate study outcomes.

### Example 4d

The above described model is applied for the combination of topiramate, levetiracetam, and ceftriaxone. The following doses are administered: Levetiracetam: 55 mg/kg/day up to a maximum of 6,000 mg/person/day, topiramate 400 mg/kg/day and ceftriaxone 4 g/day. In addition, the same combination is tested using a low dose treatment scheme of levetiracetam 30 mg/kg/day, topiramate 100 mg/day, ceftriaxone 2 g/day. Treatment shall preferably start within 8 hours of injury, but could be started also 24-72 h after injury, if 8 h is deemed not feasible Treatment shall last 1-3 months. Treatment shall be administered intravenously for the first 1-7 days of treatment. Treatment shall be administered intravenously, orally, nasogastrically or by other treatment routes after the first 1-7 days of treatment, depending on the individual health status of the patient. Patients may continue to be evaluated after completion of treatment, for total of 2 years from injury. Outcome evaluations of the study may include incidence and severity of PTE 1 year and 2 years after injury, treatment side effects, treatment discontinuation because of side effects, mortality, and blood levels of the drugs. These outcomes are compared between patients treated with drug combinations described and patients treated with placebo. Standard statistical procedures shall be used to evaluate study outcomes.

### Example 4e to 4u

In line with examples 1e to 1u, respective preferred combinations are also tested in patients, as described for examples 4a to 4d. The doses to be administered for the individual drugs for these tests to represent the highest tolerable dose in the individual patient, administered as combination are listed below. Respective doses are agmatine, 3.56 g/day; anakinra, 8 mg/kg/day; brivaracetam, 400 mg/day; ceftriaxone, 4 g/day; deferoxamine, 62 mg/kg/day up to a maximum of 6000 mg/day; fingolimod, 1.25 mg/day; gabapentin, 3,200 mg/day; ifenprodil, 40 mg/day; levetiracetam, 55 mg/kg up to a maximum of 6,000 mg/day; losartan, 100 mg/day; melatonin, 20 mg/day; memantine, 28 mg/day; N-acetylcysteine, 300 mg/kg/day; padevonil, 800 mg/day; perampanel, 24 mg/day; phenobarbital, 200 mg; sulforaphane, 60 mg/day; topiramate, 400 mg/day; valproate, 3,000 mg/day; α-tocopherol, 15 mg/day. For the low dose approach, the effective doses for each member of the fixed combination are selected to represent only 50-75% of the highest approved dose for administration in man as single drug. Respective doses are agmatine, 1.78-2.67 g/day; anakinra, 4-6 mg/kg/day; brivaracetam, 200-300 mg/day; ceftriaxone, 2-3 g/day; deferoxamine, 31-4500 mg/kg/day; fingolimod, 0.625-0.94 mg/day; gabapentin, 1600-2400 mg/day; ifenprodil, 20-30 mg/day; levetiracetam, 27.5-41 mg/kg/day up to a maximum of 3000-4500 mg/day; losartan 50-75 mg/day; melatonin, 10-15 mg/day; memantine 14-21 mg/day; N-acetylcysteine, 150-225 mg/kg/day; padsevonil, 200 mg/day; perampanel, 12-18 mg/day; phenobarbital, 100-150 mg/day; sulforaphane, 30-45 mg/day; topiramate, 100-300 mg/day; valproate, 1500-2,250 mg/day; α-tocopherol, 7.5-11.25 mg/day.

### Example 5: Preparation of a Liquid (Aqueous) Solution of Topiramate Using Meglumine Solution as Vehicle

Using purified water, meglumine (CAS-number 6284-40-8) solutions were prepared at concentrations ranging from 0.3% (w/v) to 5% (w/v) by placing the requisite amount of meglumine into a 25 mL glass measuring cylinder and adding purified water to a final volume of 25 mL. For a 0.3% (w/v) solution, 75 mg of the meglumine powder were dissolved in purified water and filled to a final volume of 25 mL solution. For a 5% (w/v) solution, 1250 mg of meglumine powder were dissolved in purified water and filled to 25 mL. Meglumine dissolved instantly in water. The solutions were stirred or shaken before use to ensure homogenous distribution.

For the preparation of the topiramate solution, topiramate was weighed into a 25 mL glass beaker in the amounts given in the table below for a volume of 10 mL. The meglumine solution is added to nearly the final volume (about 9.5 mL), and the solution is stirred using a magnetic stirrer for about 20 min, until a clear solution is obtained.

TABLE 1

| | | Maximal topiramate | pH of solution |
|---|---|---|---|
| | Meglumine | concentration | prior to pH |
| Formulation | concentration | achievable | adjustment |
| Formulation 5.1 | 0.3% | 100 mg/10 mL | About 8.4 |
| Formulation 5.2 | 0.5% | 130 mg/10 mL | About 8.5 |
| Formulation 5.3 | 1.0% | 200 mg/10 mL | About 8.5 |
| Formulation 5.4 | 2.5% | 370 mg/10 mL | About 9.0 |
| Formulation 5.5 | 5.0% | 650 mg/10 mL | About 9.5 |
| Formulation 5.6 | 0.0% | <100 mg/10 mL¹ | Not quantified |

¹dissolution was attempted with constant stirring for 30 min and application of moderate heat (50° C.). A nearly complete dissolution was achieved at the end of the 30 min stirring period, but the solution was not stable, and precipitation occurred following cooling and overnight storage at 2-8° C.

The data clearly indicate, that meglumine solubilizes topiramate in a concentration-dependent manner. The solubility of topiramate is directly proportional to the meglumine concentration, as depicted in FIG.

Following dissolution, the pH of the Formulations 5.2-5.5 were acidified by adding few drops of acetic acid or 0.1 M hydrochloric acid (HCl) or acetic acid under constant stirring, while the pH was checked continuously using a pH meter. The pH was easily modified to reach a value between 6.5 and 8.5. The amount of HCl needed was proportional to the concentration of meglumine used; preparation of the 5% (w/v) meglumine solution required the largest amount of HCL, whereas a few drops were sufficient for 10 mL of formulation 5.2. After pH adjustment, the volume was adjusted to 10 mL each using the respective meglumine vehicle solution.

The solution was then filtered through a 0.2 μm sterile filter prior to administration to ensure sterility before use. Following preparation and pH adjustment, Formulations 5.1-5.5 were found to be stable with no precipitation within 48 h if stored at room temperature or at 2-8° C. A long-term storage over 4 weeks indicates, that no precipitation occurred within 4 weeks if stored at 2-8° C. or at room temperature.

### Example 6: Preparation of an Aqueous Solution of Topiramate Using Meglumine and Polysorbate 80 Solution as Vehicle

Polysorbate 80 (CAS No. 9005-65-6) was further used in the preparation of liquid formulations of topiramate. In this example, the solubility of topiramate was examined. In addition, the solution solubility of topiramate was examined in the presence of a 0.3% (w/v) polysorbate 80 solution.

The following procedure was followed. Using purified water, a 0.3% meglumine (CAS-number 6284-40-8) and 0.3% polysorbate 80 (CAS No. 9005-65-6) solution was prepared by placing 75 mg of meglumine into a 25 mL glass measuring cylinder and adding purified water to a final volume of nearly 25 mL. Then, 75 mg polysorbate 80 were added, and the solution was carefully shaken to allow complete dissolution. The glass cylinder was filled to 25 mL with purified water. The solution was stirred or shaken before use to ensure homogeneity.

The solution above was used to prepare a 10 mg/mL topiramate solution. For this purpose, 100 mg of topiramate were added to a glass vial, and the 0.3% meglumine and 0.3% polysorbate 80 solution was added to a final volume of 10 mL. The solution was stirred until all topiramate dissolved within few minutes. The solution pH (about pH 8.0) was adjusted with a few drops of a 0.1 M HCl solution or acetic acid to the range of 6.5 to 8.5. The solution was stable for several days without crystallization or precipitation at room temperature or at 2-8° C.

### Example 7: Preparation of an Aqueous Solution of Topiramate and Levetiracetam Using Meglumine without or with Polysorbate 80 as Vehicle

The following procedure was followed. Using purified water, a 0.3% meglumine (CAS-number 6284-40-8) solution was prepared by adding 75 mg of meglumine to a 25 mL glass measuring cylinder and adding purified water to a final volume of nearly 25 mL. The glass cylinder was filled to 25 mL as final volume with purified water. In a separate vial, a 0.3% meglumine and 0.3% polysorbate 80 solution was prepared by mixing 75 mg meglumine and 75 mg polysorbate 80 with 25 mL purified water. The solutions were stirred or shaken before use to ensure homogeneity.

The solution above was used to prepare a 10 mg/mL topiramate solution containing 50-75 ng/mL levetiracetam. For this purpose, 100 mg of topiramate and 500, 670, or 750 mg of levetiracetam were added to a glass vial. Then, the 0.3% meglumine solution or the 0.3% meglumine+0.3% polysorbate 80 solution was added to a final volume of 10 mL. The solution was carefully stirred until all material dissolved. Levetiracetam dissolved rapidly, while dissolution of topiramate took a few minutes. The solution pH (close to pH 8.0) was adjusted with a few drops of a 0.1 M HCl solution or acetic acid to the range of 6.5 to 8.5. The solution was stable for several days without crystallization or precipitation at room temperature or at 2-8° C.

TABLE 2

| | Meglumine/ | | | |
|---|---|---|---|---|
| | polysorbate 80 | | | pH of solution |
| | concentration | Topiramate | Levetiracetam | prior to pH |
| Formulation | (w/v) | concentration | concentration | adjustment |
| Formulation 7.1 | 0.3%/0.0% | 100 mg/10 mL | 500 mg/10 mL | About 8.4 |
| Formulation 7.2 | 0.3%/0.0% | 100 mg/10 mL | 670 mg/10 mL | About 8.3 |
| Formulation 7.3 | 0.3%/0.0% | 100 mg/10 mL | 750 mg/10 mL | About 8.3 |
| Formulation 7.4 | 0.3%/0.3% | 100 mg/10 mL | 500 mg/10 mL | About 8.3 |
| Formulation 7.5 | 0.3%/0.3% | 100 mg/10 mL | 670 mg/10 mL | About 8.4 |
| Formulation 7.6 | 0.3%/0.3% | 100 mg/10 mL | 750 mg/10 mL | About 8.3 |

In formulations 7.1 to 7.6, levetiracetam may be replaced with brivaracetam. The doses of brivaracetam are lower than those of levetiracetam. For example, instead of 500 mg/10 mL levetiracetam, 30-50 mg/10 mL brivaracetam may be used; instead of 670 mg/10 mL levetiracetam, 45-67 mg/10 mL brivaracetam may be used; and instead of 750 mg/10 mL levetiracetam, 50-75 mg/10 mL brivaracetam may be used. Brivaracetam is water-soluble.

The formulations were stable for several days at room temperature and at 2-8° C. No precipitation was observed in the meglumine solutions with or without polysorbate 80.

### Example 8: Preparation of an Aqueous Solution of Topiramate and Levetiracetam and Atorvastatin Sodium Including a Vehicle Containing Meglumine with or without Polysorbate 80

In a further experiment, solutions containing topiramate (10 mg/mL), levetiracetam (75 mg/mL), and atorvastatin sodium, (3.47 mg/mL), corresponding to an atorvastatin concentration of 3.33 mg/mL, were prepared. The solutions included 3% (w/v) meglumine solution as well as the 0.3% (w/v) meglumine+0.3% (w/v) polysorbate 80 solution was evaluated.

The meglumine and meglumine+polysorbate 80 solutions were prepared as described in Example 7.

In two separate glass beakers, were mixed 100 mg topiramate, 750 mg levetiracetam, and 34.7 mg atorvastatin sodium. The meglumine solution was added to the first beaker to a total volume of 10 mL, and the meglumine+polysorbate 80 solution was added to the second beaker to a total volume of 10 mL. In the first beaker, solids dissolved after about 20 min of stirring. In the second beaker, the dissolution was faster, and the liquid became clear after about 5 min stirring. In two follow-up experiments, the atorvastatin concentration was reduced to 2 mg/mL. This resulted in an overall faster dissolution for both formulations.

TABLE 3

| | Meglumine/ | | | | |
|---|---|---|---|---|---|
| | polysorbate 80 | Topiramate | Levetiracetam | Atorvastatin | pH of solution |
| | concentration | concentration | concentration | concentration | prior to pH |
| Formulation | (w/v) | (mg/10 mL) | (mg/10 mL) | (mg/10 mL) | adjustment |
| Formul. 8.1 | 0.3%/0.0% | 100 | 500 | 33.3 | About 8.9 |
| Formul. 8.2 | 0.3%/0.3% | 100 | 670 | 33.3 | About 8.9 |
| Formul. 8.3 | 0.3%/0.0% | 100 | 500 | 20 | About 8.7 |
| Formul. 8.4 | 0.3%/0.3% | 100 | 670 | 20 | About 8.6 |
| Formul. 8.5 | 0.0/0.0 | — | — | 33.3 | Not measured |
| Formul. 8.6 | 0.5%/0.0% | 100 | 500 | 33.3 | About 9.2 |
| Formul. 8.7 | 1.0%/0.0% | 100 | 500 | 33.3 | About 9.8 |
| Formul. 8.8 | 2.5%/0.0% | 100 | 500 | 33.3 | About 10.4 |
| Formul. 8.9 | 0.3%/0.1% | 100 | 670 | 33.3 | About 8.9 |

After about 20 h of storage under ambient conditions, precipitation was observed in formulations 8.1, 8.6, 8.7, and 8.8 independently from the meglumine concentration. These observations indicate that meglumine did not improve the solubility of atorvastatin. Similar precipitation was also noted in formulation 8.5, indicating that atorvastatin, if dissolved in water, precipitates after about 20 h.

Polysorbate 80 (formulation 8.2 and 8.4) improved the solution stability. While no precipitation was observed for 48 hours in formulation 8.2, followed by slight precipitation, in formulation 8.4, with the reduced content of atorvastatin, the solution remained stable for several days at room temperature. The magnitude of the effect of the addition of polysorbate 80 at 0.1% was less pronounced. While it did improve the dissolution speed, first precipitation was again seen after about 24 h (formulation 8.9).

In formulations 8.1 to 8.4 and 8.6 to 8.9, levetiracetam may be replaced with brivaracetam. The doses of brivaracetam are typically lower than those of levetiracetam. For example, instead of 500 mg/10 mL levetiracetam, 30-50 mg/10 mL brivaracetam may be used; instead of 670 mg/10 mL levetiracetam, 45-67 mg/10 mL brivaracetam may be used; and instead of 750 mg/10 mL levetiracetam, 50-75 mg/10 mL brivaracetam may be used. Brivaracetam is water-soluble.

The pH of formulation 8.1, 8.2, 8.3, 8.3., and 8.9 was adjusted to 6.5-8.5 with a few drops of 0.1% HCl or acetic acid. The pH of formulations 8.6, 8.7, and 8.8 was adjusted to about 8.5.

### Example 9

Formulations 9.1-9.10 shown in Table 4 may be prepared by combining topiramate, levetiracetam, and atorvastatin in requisite proportions. The formulations may be prepared as an aqueous solution containing 0.3% (w/v) or more of meglumine, as, e.g., about 0.3% (w/v) meglumine may be used to dissolve topiramate at a concentration of 100 mg/mL. For example, meglumine concentration may be, e.g., 0.3% (w/v) to 2.5% (w/v). The polysorbate 80 concentration can be varied from 0.0% (i.e., no polysorbate 80) to 1% (w/v), to facilitate dissolution rate and the stability of the resulting solution. Atorvastatin is an optional component of the formulations.

In cases, where a higher dosage volume is preferred, the formulations may be prepared in a higher volume, e.g., 20 or 25 mL instead of 10 mL. Alternatively, where a higher dosage volume is preferred, the formulations may be prepared in a higher volume, e.g., up to 250 mL (e.g., 20 mL, 25 mL, 40 mL, 50 mL, 60 mL, 75 mL, 80 mL, 100 mL, 120 mL, 125 mL, 140 mL, 150 mL, 160 mL, 175 mL, 180 mL, 200 mL, 220 mL, 225 mL, 240 mL, or 250 mL). The amount of each compound may be proportionally reduced, and the concentration of meglumine may be also reduced proportionally. For example, for such a high-volume formulation, the active ingredients in formulation 9.1 may be, e.g.:
Topiramate: 100 mg/20 mL or 100 mg/25 mL;
Levetiracetam: 750 mg/20 mL or 750 mg/25 mL; and
Atorvastatin: 20 mg/20 mL or 20 mg/25 mL.

Alternatively, the formulation may be diluted after preparation with an acceptable diluent, e.g. purified water or purified water containing 0.1-1% (w/v) meglumine.

In each case, the pH may be adjusted to a range of 5.5 to 8.5, preferably, 6.5 to 8.5.

TABLE 4

| | | | | Doses per | |
|---|---|---|---|---|---|
| | Topiramate | Levetiracetam | Atorvastatin | administration: | Dosing |
| | (TPM) | (LEV) | (ATO) | TPM/LEV/ATO* | volume |
| Example 9.1 | 100 mg/10 mL | 750 mg/10 mL | 20 mg/10 mL | 200/1500/40 bid | 20 mL |
| Example 9.2 | 100 mg/10 mL | 750 mg/10 mL | 16 mg/10 mL | 200/1500/32 bid | 20 mL |
| Example 9.3 | 100 mg/10 mL | 1000 mg/10 mL | 20 mg/10 mL | 200/2000/40 bid | 20 mL |
| Example 9.4 | 100 mg/10 mL | 1000 mg/10 mL | 16 mg/10 mL | 200/2000/32 bid | 20 mL |
| Example 9.5 | 100 mg/10 mL | 800 mg/10 mL | 16 mg/10 mL | 250/2000/40 bid | 25 mL |
| Example 9.6 | 100 mg/10 mL | 800 mg/10 mL | 12.8 mg/10 mL | 250/2000/32 bid | 25 mL |
| Example 9.7 | 100 mg/10 mL | 1200 mg/10 mL | 16 mg/10 mL | 250/3000/40 bid | 25 mL |
| Example 9.8 | 100 mg/10 mL | 1200 mg/10 mL | 12.8 mg/10 mL | 250/3000/32 bid | 25 mL |
| Example 9.9 | 100 mg/10 mL | 600 mg/10 mL | 16 mg/10 mL | 250/1500/40 bid | 25 mL |
| Example 9.10 | 100 mg/10 mL | 600 mg/10 mL | 12.8 mg/10 mL | 250/1500/32 bid | 25 mL |

*the units are mg.

### Example 10

Formulations 10.1-10.10 may be prepared by combining topiramate, brivaracetam, and atorvastatin may by combined in requisite proportions. The formulations may be prepared as an aqueous solution containing 0.3% (w/v) or more of meglumine, as, e.g., about 0.3% (w/v) of meglumine may be used to dissolve topiramate at 100 mg/mL. For example, meglumine concentration may be, e.g., 0.3% (w/v) to 2.5% (w/v). The polysorbate 80 concentration can be varied from 0.0% (no polysorbate 80) to 1% (w/v).

In cases, where a higher dosage volume is preferred, the formulations may be prepared in a higher volume, e.g., 20 or 25 mL instead of 10 mL. Alternatively, where a higher dosage volume is preferred, the formulations may be prepared in a higher volume, e.g., up to 250 mL (e.g., 20 mL, 25 mL, 40 mL, 50 mL, 60 mL, 75 mL, 80 mL, 100 mL, 120 mL, 125 mL, 140 mL, 150 mL, 160 mL, 175 mL, 180 mL, 200 mL, 220 mL, 225 mL, 240 mL, or 250 mL). The amount of each compound may be proportionally reduced, and the concentration of meglumine may be also reduced proportionally. For example, for such a high-volume formulation, the active ingredients in formulation 10.1 may be, e.g.:
Topiramate: 100 mg/20 mL or 100 mg/25 mL;
Brivaracetam: 50 mg/20 mL or 50 mg/25 mL;
Atorvastatin: 20 mg/20 mL or 20 mg/25 mL.

Alternatively, the formulation may be diluted after preparation with an acceptable diluent, e.g. purified water or purified water containing 0.1-1% (w/v) meglumine.

In each case, the pH may be adjusted to a range of 5.5 to 8.5, preferably 6.5 to 8.5.

TABLE 5

| | | | | Doses per | |
|---|---|---|---|---|---|
| | Topiramate | Brivaracetam | Atorvastatin | administration: | Dosing |
| | (TPM) | (BRI) | (ATO) | TPM/BRI/ATO* | volume |
| Example 10.1 | 100 mg/10 mL | 50 mg/10 mL | 20 mg/10 mL | 200/100/40 bid | 20 mL |
| Example 10.2 | 100 mg/10 mL | 50 mg/10 mL | 16 mg/10 mL | 200/100/32 bid | 20 mL |
| Example 10.3 | 100 mg/10 mL | 65 mg/10 mL | 20 mg/10 mL | 200/162/40 bid | 20 mL |
| Example 10.4 | 100 mg/10 mL | 65 mg/10 mL | 16 mg/10 mL | 200/162/32 bid | 20 mL |
| Example 10.5 | 100 mg/10 mL | 55 mg/10 mL | 16 mg/10 mL | 250/137/40 bid | 25 mL |
| Example 10.6 | 100 mg/10 mL | 55 mg/10 mL | 12.8 mg/10 mL | 250/137/32 bid | 25 mL |
| Example 10.7 | 100 mg/10 mL | 80 mg/10 mL | 16 mg/10 mL | 250/200/40 bid | 25 mL |
| Example 10.8 | 100 mg/10 mL | 80 mg/10 mL | 12.8 mg/10 mL | 250/200/32 bid | 25 mL |
| Example 10.9 | 100 mg/10 mL | 40 mg/10 mL | 16 mg/10 mL | 250/100/40 bid | 25 mL |
| Example 10.10 | 100 mg/10 mL | 40 mg/10 mL | 12.8 mg/10 mL | 250/100/32 bid | 25 mL |

*the units are mg.

This application claims benefit of U.S. Provisional Application No. 62/835,707, filed Apr. 18, 2019, and U.S. Provisional Application No. 62/926,130, filed Oct. 25, 2019, the contents of which are incorporated herein by reference in their entirety.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments.

Other embodiments are in the claims.

What is claimed is:

1. A liquid pharmaceutical composition comprising topiramate or a pharmaceutically acceptable salt thereof, meglumine, and a pharmaceutically acceptable excipient.

2. The liquid pharmaceutical composition of claim 1, wherein the composition comprises 1 mg/mL to 550 mg/mL of meglumine.

3. The liquid pharmaceutical composition of claim 1, wherein the composition comprises 1 mg/mL to 100 mg/mL of topiramate or a pharmaceutically acceptable salt thereof.

4. The liquid pharmaceutical composition of claim 1, wherein the composition further comprises levetiracetam.

5. The liquid pharmaceutical composition of claim 4, wherein the composition comprises 5 mg/mL to 500 mg/mL of levetiracetam.

6. The liquid pharmaceutical composition of claim 4, wherein the weight ratio of levetiracetam to topiramate to is 5:1 to 15:1.

7. The liquid pharmaceutical composition of claim 1, wherein the composition further comprises brivaracetam.

8. The liquid pharmaceutical composition of claim 7, wherein the composition comprises 0.5 mg/mL to 50 mg/mL of brivaracetam.

9. The liquid pharmaceutical composition of claim 7, wherein the weight ratio of brivaracetam to topiramate to is 1:4 to 1:1.

10. The liquid pharmaceutical composition of claim 1, wherein the composition further comprises atorvastatin or a pharmaceutically acceptable salt thereof.

11. The liquid pharmaceutical composition of claim 10, wherein the composition comprises 0.1 mg/mL to 80 mg/mL atorvastatin or a pharmaceutically acceptable salt thereof.

12. The liquid pharmaceutical composition of claim 10, wherein the weight ratio of topiramate to atorvastatin is 5:1 to 15:1.

13. The liquid pharmaceutical composition of claim 1, wherein the composition has a pH of 5.5 to 8.8.

14. The liquid pharmaceutical composition of claim 1, wherein the composition further comprises an acidulant.

15. The liquid pharmaceutical composition of claim 1, wherein the acidulant is acetic acid.

16. The liquid pharmaceutical composition of claim 1, wherein the composition further comprises a calcium-scavenging agent.

17. The liquid pharmaceutical composition of claim 16, wherein the calcium-scavenging agent is EDTA, EGTA, BAPTA, or an alkali salt thereof.

18. The liquid pharmaceutical composition of claim 1, wherein the composition further comprises an emulsifier.

19. The liquid pharmaceutical composition of claim 18, wherein the composition comprises 0.001% to 5.0% (w/v) of the emulsifier.

20. The liquid pharmaceutical composition of claim 18, wherein the emulsifier is polyoxyethylene (20) sorbitan monooleate (polysorbate 80).

21. The liquid pharmaceutical composition of claim 1, wherein the composition is aqueous.

22. A kit comprising a first container and a second container, the first container comprising topiramate or a pharmaceutically acceptable salt thereof, and the second container comprising a pharmaceutically acceptable aqueous solution of meglumine.

23. A method of treating a patient in need thereof, the method comprising administering to the patient a therapeutically effective amount of the composition of claim 1.

24. A method of treating a patient in need thereof, the method comprising combining the first container contents and the second container contents in the kit of claim 33 to produce a liquid pharmaceutical composition, and administering a therapeutically effective amount of the liquid pharmaceutical composition to the patient.

25. The method of claim 24, wherein the therapeutically effective amount is an amount providing 0.5 mg/kg/day to 20 mg/kg/day of topiramate.

26. The method of claim 24, wherein the therapeutically effective amount is an amount providing at least 40 mg/day to 1200 mg/day of topiramate.

27. The method of claim 24, wherein the therapeutically effective amount is an amount providing 2.5 mg/kg/day to 150 mg/kg/day of levetiracetam.

28. The method of claim 24, wherein the therapeutically effective amount is an amount providing 0.2 mg/kg/day to 10 mg/kg/day of brivaracetam.

29. The method of claim 24, wherein the therapeutically effective amount is an amount providing 0.1 to 2.0 mg/kg/day of atorvastatin.

30. The method of claim 24, wherein the liquid pharmaceutical composition is administered parenterally or orally.

31. The method of claim 24, wherein the patient is in need of a treatment for a disorder or condition selected from the group consisting of epilepsy, seizures, anoxia, stroke, traumatic brain injury, brain infection, brain abscess, aneurysm, subarachnoid hemorrhage, status epilepticus, refractory status epilepticus, refractory partial onset seizures (POS), gambling addiction, migraines, substance dependence, alcoholism, cocaine dependence, opioid addiction, nicotine dependence, metabolic syndrome X, diabetes mellitus, type 2, vomiting, obsessive-compulsive disorder, refractory generalized social phobia, Tourette Syndrome, levodopa-induced dyskinesia in Parkinson's Disease, Prader-Willi syndrome, multiple sclerosis, Lennox-Gastaut Syndrome, Dravet's syndrome, bipolar disorder, obesity, post-traumatic stress disorder, headaches, and conditions caused by exposure to a chemical warfare nerve agent.

32. The method of claim 24, wherein the patient is in need of neuroprotection.

33. The method of claim 24, wherein the patient is in need of a treatment for a disorder or condition selected from the group consisting of traumatic brain injury, stroke, and a brain infection.

34. The method of claim 24, wherein the patient is in need of a treatment for a brain abscess.

35. A method of treating epileptogenesis in a subject after brain insult, the method comprising administering to the subject a therapeutically effective amount of a therapeutic combination comprising two to five drugs selected from the group consisting of anti-inflammatory drugs, antioxidant drugs, neuroprotective drugs, GABA-potentiating drugs, glutamate-suppressing drugs, drugs with presynaptic effects on neuronal excitability, drugs having a metabolic mode of action, and pharmaceutically acceptable salts thereof, provided that the two to five drugs are all different.

36. The method of claim 35, wherein each anti-inflammatory drug is independently ibuprofen, celecoxib, parecoxib, a sartan, atorvastatin, fingolimod, anakinra, or agmatine.

37. The method of claim 35, wherein each antioxidant drug is independently α-tocopherol, deferoxamine, N-acetylcysteine, sulforaphane, or melatonin.

38. The method of claim 35, wherein each neuroprotective drug is independently gabapentin, pregabalin, ifenprodil, perampanel, memantine, agmatine, celecoxib, or ceftriaxone.

39. The method of claim 35, wherein each GABA-potentiating drug and each glutamate-suppressing drug is independently topiramate, valproate, phenobarbital, deferoxamine, ceftriaxone, ifenprodil, perampanel, or memantine.

40. The method of claim 35, wherein each drug with presynaptic effects on neuronal excitability is independently levetiracetam, brivaracetam, etiracetam, padsevonil, gabapentin, pregabalin, or valproate.

41. The method of claim 35, wherein each drug having a metabolic mode of action is independently stiripentol, 2-deoxy-D-glucose, 5-azacitidine, decitabine, β-hydroxybutyrate, or vorinostat.

42. The method of claim 35, wherein the therapeutic combination is a combination of topiramate, levetiracetam, and deferoxamine or a pharmaceutically acceptable salt thereof.

43. The method of claim 35, wherein the therapeutic combination is a combination of topiramate, levetiracetam, and atorvastatin or a pharmaceutically acceptable salt thereof.

44. The method of claim 35, wherein the therapeutic combination is a combination of topiramate and levetiracetam.

45. The method of claim 35, wherein the therapeutic combination is a combination of topiramate, levetiracetam, and ceftriaxone or a pharmaceutically acceptable salt thereof.

46. The method of claim 35, wherein the therapeutic combination is a combination of topiramate, levetiracetam, and gabapentin or a pharmaceutically acceptable salt thereof.

47. The method of claim 35, wherein the therapeutic combination is a combination of topiramate, levetiracetam, and pregabalin or a pharmaceutically acceptable salt thereof.

48. The method of claim 35, wherein the therapeutic combination is s a combination of levetiracetam, topiramate, and α-tocopherol.

49. The method of claim 35, wherein the therapeutic combination is a combination of levetiracetam, deferoxamine or a pharmaceutically acceptable salt thereof, and melatonin.

50. The method of claim 35, wherein the therapeutic combination is a combination of levetiracetam, deferoxamine or a pharmaceutically acceptable salt thereof, and celecoxib.

51. The method of claim 35, wherein the therapeutic combination is a combination of levetiracetam, deferoxamine or a pharmaceutically acceptable salt thereof, gabapentin or a pharmaceutically acceptable salt thereof, and fingolimod or a pharmaceutically acceptable salt thereof.

52. The method of claim 35, wherein the therapeutic combination is a combination of levetiracetam, atorvastatin or a pharmaceutically acceptable salt thereof, and ceftriaxone.

53. The method of claim 52, wherein at least one of levetiracetam, atorvastatin or a pharmaceutically acceptable salt thereof, and ceftriaxone is administered at a low dose.

54. The method of claim 53, wherein each of levetiracetam, atorvastatin or a pharmaceutically acceptable salt thereof, and ceftriaxone is administered at a low dose.

55. The method of claim 35, wherein the therapeutic combination is a combination of levetiracetam and perampanel or a pharmaceutically acceptable salt thereof.

56. The method of claim 35, wherein the therapeutic combination is a combination of levetiracetam, perampanel or a pharmaceutically acceptable salt thereof, and ceftriaxone.

57. The method of claim 35, wherein the therapeutic combination is a combination of levetiracetam, parecoxib, and anakinra.

58. The method of claim 35, wherein the therapeutic combination is a combination of levetiracetam and phenobarbital.

59. The method of claim 35, wherein the therapeutic combination is a combination of brivaracetam, and topiramate.

60. The method of claim 35, wherein the therapeutic combination is a combination of brivaracetam, topiramate, and ceftriaxone.

61. The method of claim 35, wherein the therapeutic combination is a combination of brivaracetam and perampanel.

62. The method of claim 35, wherein the therapeutic combination is a combination of brivaracetam, perampanel, and ceftriaxone.

63. The method of claim 35, wherein the therapeutic combination is a combination of valproate or a pharmaceutically acceptable salt thereof, losartan or a pharmaceutically acceptable salt thereof, and memantine or a pharmaceutically acceptable salt thereof.

64. The method of claim 35, wherein the therapeutic combination comprises three of the drugs.

65. The method of claim 35, wherein the dose of each drug in the combination is the highest tolerable dose in the individual patient, when the drug is administered in the therapeutic combination.

66. The method of claim 35, wherein the therapeutic combination is initially administered to the subject intravenously for 1 to 30 days.

67. The method of claim 35, wherein the therapeutic combination is administered to the subject intramuscularly, subcutaneously, orally, percutaneously, sublingually, buccally, intranasally, by inhalation, or rectally.

68. The method of claim 35, wherein, after the therapeutic combination administration is initiated, the therapeutic combination administration is maintained by prolonged oral or parenteral administration.

69. The method of claim 68, wherein the therapeutic combination administration is maintained for 3 to 6 months following the brain insult.

70. The method of claim 35, wherein at least two drugs are present in the same pharmaceutical composition.

71. The method of claim 35, wherein at least three drugs are present in the same pharmaceutical composition.

72. The method of claim 35, where the administering step is initiated within 7 days after the brain insult.

73. The method of claim 72, wherein the administering step is initiated within 48 h after the brain insult.

74. The method of claim 72, wherein the administering step is initiated within 24 h after the brain insult.

75. The method of claim 72, wherein the administering step is initiated within 8 h after the brain insult.

76. The method of claim 35, wherein the therapeutic combination is administered to the subject for a period of 3 day to 3 months after the insult.

77. The method of claim 76, wherein the therapeutic combination is administered to the subject for 5-30 days after the brain insult.
